SEQUENCE LISTING (12 sequences)

SEQ ID NO: 1 - protein, 123 aa, Homo sapiens
QVQLVESGGGVVQPGRSLRLSCAASGFRFTTHGMHWVRQAPGKGLEWVAVISYNGRNKYYADYVNGRFTISRDDSRDTVFLQMNSLRPEDTAMYYCAKVRGDGYGDYGYFDYWGHGTLVSVSS

SEQ ID NO: 2 - DNA, 369 bp, Homo sapiens
caggtgcagctggtggagtctgggggaggcgtggtccagcctgggaggtccctaagactctcctgtgcagcctctggattcaggttcactacacacggcatgcactgggtccgccaggctccaggcaaggggctggagtgggtggcagttatatcatataatggaagaaacaaatactatgcagactacgtgaacggccgattcaccatctccagagacgattccagggacacggtgtttctgcaaatgaacagcctgagacctgaggacacggctatgtactactgtgcgaaagttaggggcgatggctacggtgactatggctactttgactactggggccacggaaccctggtcagcgtctcctca

SEQ ID NO: 3 - protein, 123 aa, Homo sapiens
EVQLVESGGGVVQPGRSLRLSCAASGFRFTTHGMHWVRQAPGKGLEWVAVISYNGRNKYYADYVNGRFTISRDDSRDTVFLQMNSLRPEDTAMYYCAKVRGDGYGDYGYFDYWGHGTLVSVSS

SEQ ID NO: 4 - DNA, 369 bp, Homo sapiens
gaggtgcagctggtggagtctgggggaggcgtggtccagcctgggaggtccctaagactctcctgtgcagcctctggattcaggttcactacacacggcatgcactgggtccgccaggctccaggcaaggggctggagtgggtggcagttatatcatataatggaagaaacaaatactatgcagactacgtgaacggccgattcaccatctccagagacgattccagggacacggtgtttctgcaaatgaacagcctgagacctgaggacacggctatgtactactgtgcgaaagttaggggcgatggctacggtgactatggctactttgactactggggccacggaaccctggtcagcgtctcctca

SEQ ID NO: 5 - protein, 123 aa, Homo sapiens
EVQLVESGGGLVQPGGSLRLSCAASGFRFTTHGMHWVRQAPGKGLEWVAVISYNGRNKYYADYVNGRFTISRDDSRDTVFLQMNSLRPEDTAMYYCAKVRGDGYGDYGYFDYWGHGTLVSVSS

SEQ ID NO: 6 - DNA, 369 bp, Homo sapiens
gaagtgcagctggtggaaagcggcggcggcctggtgcagccgggcgggtccctgagactctcctgtgcagcctctggattcaggttcactacacacggcatgcactgggtccgccaggctccaggcaaggggctggagtgggtggcagttatatcatataatggaagaaacaaatactatgcagactacgtgaacggccgattcaccatctccagagacgattccagggacacggtgtttctgcaaatgaacagcctgagacctgaggacacggctatgtactactgtgcgaaagttaggggcgatggctacggtgactatggctactttgactactggggccacggaaccctggtcagcgtctcctca

SEQ ID NO: 7 - protein, 123 aa, Homo sapiens
QVQLVESGGGVVQPGRSLRLSCAASGFRFTTHGMHWVRQAPGKGLEWVAVISYNGRNKYYADYVNGRFTISRDDSRDTVFLQMNSLRPEDTAMYYCAKVRGDGYGDYGYFDYWGHGTLVTVSS

SEQ ID NO: 8 - DNA, 369 bp, Homo sapiens
caggtgcagctggtggagtctgggggaggcgtggtccagcctgggaggtccctaagactctcctgtgcagcctctggattcaggttcactacacacggcatgcactgggtccgccaggctccaggcaaggggctggagtgggtggcagttatatcatataatggaagaaacaaatactatgcagactacgtgaacggccgattcaccatctccagagacgattccagggacacggtgtttctgcaaatgaacagcctgagacctgaggacacggctatgtactactgtgcgaaagttaggggcgatggctacggtgactatggctactttgactactggggccacggaaccctggtcaccgtctcctca

SEQ ID NO: 9 - protein, 106 aa, Homo sapiens
QSALTQPRSVSGSPGQSVTISCTGTYNYVSWYQQHPGKAPKLMIFDVSRRSSGVPDRFSGSKSGNTASLTISGLQAEDEADYYCCSYGGTYLYVFGTGTTVTVLGQ

SEQ ID NO: 10 - DNA, 318 bp, Homo sapiens
cagtctgccctgactcagcctcgctcagtgtccgggtctcctggacagtcagtcaccatctcctgcactggaacttataactatgtctcctggtaccaacagcacccaggcaaagcccccaaactcatgatttttgatgtcagtaggcggtcctcaggggtccctgatcgcttctctggctccaagtctggcaacacggcctccctgaccatctctgggctccaggctgaggatgaggctgattattactgctgctcatatggaggcacctacctttatgtcttcggaactgggactacggtcaccgtccttggtcag

SEQ ID NO: 11 - protein, 592 aa, Homo sapiens
MSDSGSYGQSGGEQQSYSTYGNPGSQGYGQASQSYSGYGQTTDSSYGQNYSGYSSYGQSQSGYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGRGRGGYDKDGRGPMTGSSGGDRGGFKNFGGHRDYGPRTDADSESDNSDNNTIFVQGLGEGVSTDQVGEFFKQIGIIKTNKKTGKPMINLYTDKDTGKPKGEATVSFDDPPSAKAAIDWFDGKEFHGNIIKVSFATRRPEFMRGGGSGGGRRGRGGYRGRGGFQGRGGDPKSGDWVCPNPSCGNMNFARRNSCNQCNEPRPEDSRPSGGDFRGRGYGGERGYRGRGGRGGDRGGYGGDRSGGGYGGDRSSGGGYSGDRSGGGYGGDRSGGGYGGDRGGGYGGDRGGGYGGDRGGGYGGDRGGYGGDRGGGYGGDRGGYGGDRGGYGGDRGGYGGDRGGYGGDRSRGGYGGDRGGGSGYGGDRSGGYGGDRSGGGYGGDRGGGYGGDRGGYGGKMGGRNDYRNDQRNRPY

SEQ ID NO: 12 - protein, 589 aa, Homo sapiens
MSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGRGRGGYDKDGRGPMTGSSGGDRGGFKNFGGHRDYGPRTDADSESDNSDNNTIFVQGLGEGVSTDQVGEFFKQIGIIKTNKKTGKPMINLYTDKDTGKPKGEATVSFDDPPSAKAAIDWFDGKEFHGNIIKVSFATRRPEFMRGGGSGGGRRGRGGYRGRGGFQGRGGDPKSGDWVCPNPSCGNMNFARRNSCNQCNEPRPEDSRPSGGDFRGRGYGGERGYRGRGGRGGDRGGYGGDRSGGGYGGDRSSGGGYSGDRSGGGYGGDRSGGGYGGDRGGGYGGDRGGGYGGDRGGGYGGDRGGYGGDRGGGYGGDRGGYGGDRGGYGGDRGGYGGDRGGYGGDRSRGGYGGDRGGGSGYGGDRSGGYGGDRSGGGYGGDRGGGYGGDRGGYGGKMGGRNDYRNDQRNRPY

(19) United States
(12) Patent Application Publication
(10) Pub. No.: US 2009/0291083 A1
(43) Pub. Date: Nov. 26, 2009

(54) BARB4 TARGET, ANTIBODY DESIGNATED BARB4, BARB4 RELATED ANTIBODIES, AND METHODS OF MAKING AND USING SAME

(75) Inventors: Heinz Peter Vollmers, Wurzburg (DE); Stephanie ute Brandlein, Zelllingen (DE); Leodevico L. Ilag, Balwyn (AU); Barbara Power, Blackburn (AU); Frank Hensel, Wurzburg (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: PATRYS LIMITED, Melbourne (AU)

(21) Appl. No.: 12/350,072

(22) Filed: Jan. 7, 2009

Related U.S. Application Data
(60) Provisional application No. 61/019,502, filed on Jan. 7, 2008.
(60) Provisional application No. 61/023,345, filed on Jan. 24, 2008.
(60) Provisional application No. 61/043,950, filed on Apr. 10, 2008.
(60) Provisional application No. 61/084,814, filed on Jul. 30, 2008.

(51) Int. Cl. A61K 39/395; C07K 16/00; C12N 5/10; C07K 14/00; A61P 35/00

(52) U.S. Cl. 424/139.1; 530/387.9; 435/325; 530/350

(57) ABSTRACT

The invention provides antibodies, functional fragments, modified and variant forms, antibody targets, nucleic acid and other compositions. Antibodies, functional fragments, modified and variant forms, nucleic acid and other compositions are useful in treatment, diagnostic and vaccination methods. One treatment method includes inhibiting growth or proliferation of proliferating cells, such as hyperproliferative cells or inducing regression of hyperproliferative cells, such as cells of a cellular hyperproliferative disorder.

### RELATED APPLICATIONS

This application claims priority to application Ser. No. 61/019,502, filed Jan. 7, 2008, application Ser. No. 61/023,345, filed Jan. 24, 2008, application Ser. No. 61/043,950, filed Apr. 10, 2008, and application Ser. No. 61/084,814, filed Jul. 30, 2008, each of which are incorporated by reference in their entirety.

### FIELD OF THE INVENTION

The invention relates to an antibody, known as BARB4, and an antigen, denoted BARB4 target. The antibody denoted BARB4 binds to BARB4 target. BARB4 target has sequence identity with TATA-binding protein-associated factor 15 (TAF 15 polypeptide). BARB4 is an IgG and binds to different types of neoplasia, cancer, tumor and metastasis. BARB4 inhibits growth of various types of cancer cells and stimulates or induces apoptosis of various types of cancer cells.

### INTRODUCTION

There is a need for methods of treating, screening for and diagnosing undesirable or aberrant cell proliferation, such as cellular hyperproliferative disorders, including neoplasias, tumors, cancers and metastasis. The invention addresses this need and provides related benefits.

### SUMMARY

The invention provides isolated and purified antigen, denoted as BARB4 target, and antigen subsequences, which have sequence identity with TATA-binding protein-associated factor 15 (TAF 15 polypeptide). The invention also provides isolated and purified antibodies and functional fragments (e.g., BARB4 antibody, as represented by antibody produced by hybridoma DSMZ Deposit No. DSM ACC2876, deposited on Dec. 19, 2007, or heavy and light chain sequences set forth as SEQ ID NOs: 1, 3, 5, 7; and 9, respectively) that bind to an antigen, denoted as BARB4 target.

Invention antigen, denoted as BARB4 target, includes amino acid sequences with complete (100%) or partial (e.g., 60%, 70%, 80%, 90%, 95% or more) identity with TATA-binding protein-associated factor 15 (TAF 15 polypeptide isoforms 1 (NP_{—}631961) and 2 (NP_{—}0034780), e.g., as set forth in SEQ ID NO:11 and 12:

| | |
|---|---|
| 1 | msdsgsygqsggeqqsystygnpgsqgygqasqsysgygqttdssygqnysgyssygqsq |
| 61 | sgysqsyggyenqkqssysqqpynnqgqqqnmessgsqggrapsydqpdygqqdsydqqs |
| 121 | gydqhqgsydeqsnydqqhdsysqnqqsyhsqrenyshhtqddrrdvsrygednrgyggs |
| 181 | qgggrgrggydkdgrgpmtgssggdrggfknfgghrdygprtdadsesdnsdnntifvqg |
| 241 | lgegvstdqvgeffkqigiiktnkktgkpminlytdkdtgkpkgeatvsfddppsakaai |
| 301 | dwfdgkefhgniikvsfatrrpefmrgggsgggrrgrggyrgrggfqgrggdpksgdwvc |
| 361 | pnpscgnmnfarrnscnqcneprpedsrpsggdfrgrgyggergyrgrggrggdrggygg |
| 421 | drsgggyggdrssgggysgdrsgggyggdrsgggyggdrgggyggdrgggyggdrgggyg |
| 481 | gdrggyggdrgggyggdrggyggdrggyggdrggyggdrggyggdrsrgg yggdrgggsg |
| 541 | yggdrsggyggdrsgggyggdrgggyggdrggyggkmggrndyrndqrnrpy |
| 1 | msdsgsygqsggeqqsystygnpgsqgygqasqsysgygqttdssygqnysgyssygqsy |
| 61 | sqsyggyenqkqssysqqpynnqgqqqnmessgsqggrapsydqpdygqqdsydqqsgyd |
| 121 | qhqgsydeqsnydqqhdsysqnqqsyhsqrenyshhtqddrrdvsrygednrgyggsqgg |
| 181 | grgrggydkdgrgpmtgssggdrggfknfgghrdygprtdadsesdnsdnntifvqglge |
| 241 | gvstdqvgeffkqigiiktnkktgkpminlytdkdtgkpkgeatvsfadppsakaaidwf |
| 301 | dgkefhgniikvsfatrrpefmrgggsgggrrgrggyrgrggfqgrggapksgdwvcpnp |
| 361 | scgnmnfarrnscnqcneprpedsrpsggdfrgrgyggergyrgrggrggdrggyggdrs |
| 421 | gggyggdrssgggysgdrsgggyggdrsgggyggdrgggyggdrgggyggdrgggyggdr |
| 481 | ggyggdrgggyggdrggyggdrggyggdrggyggdrggyggdrsrggyggdrgggsgygg |
| 541 | drsggyggdrsgggyggdrgggyggdrggyggkmggrndyrndqrnrpy |

In one embodiment, an antigen or subsequence thereof is a cell membrane bound isoform of a TAF 15 polypeptide, with complete (100%) or partial (e.g., 60%, 70%, 80%, 90%, 95% or more) identity to TATA-binding protein-associated factor 15 set forth in SEQ ID NO:11 or 12. In another embodiment, an antigen (BARB4 target) or subsequence thereof has a sequence that is completely (100%) or partially (e.g., 60%, 70%, 80%, 90%, 95% or more) identical to 10, 20, 30, 40, 50, 60, 70, 80, 90 or 100 or more contiguous amino acids in SEQ ID NO:11 or 12. In further embodiments, an antigen (BARB4 target) or subsequence thereof includes a carbohydrate moiety (e.g., an N-linked carbohydrate moiety or an O-linked carbohydrate moiety), or is expressed by a tumor or cancer cell (e.g., a pancreas cancer or tumor cell, a colon cancer or tumor cell, or a stomach cancer or tumor cell, such as BXPC-3 cells, HT-29 cells or 23132/87 cells). In additional embodiments, an antigen (BARB4 target) or subsequence thereof is an antigen to which a BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In still further embodiments, an antigen (BARB4 target) has a molecular weight of about 70-85 KDa, as determined by sodium dodecyl sulfate polyacrylamide gel electrophoresis (SDS-PAGE). In an additional embodiment, an antigen (BARB4 target) includes a fusion protein resulting from a chromosomal translocation of or into a gene encoding the TAF 15 polypeptide. In still further embodiments, an antigen (BARB4 target) is not a fusion protein resulting from a chromosomal translocation of or into a gene encoding the TAF 15 polypeptide (e.g., not a fusion protein resulting from a chromosomal translocation of a TAF 15 fused to a nuclear receptor, NOR1). In a particular aspect, isolated or purified antigen is mammalian (e.g., human).

Invention antibodies include, for example, antibodies and functional fragments that bind to an antigen (BARB4 target, such as a cell membrane bound isoform of a TAF 15 polypeptide). In one embodiment, an antibody or functional fragment competes for binding to a cell or to an antigen (e.g., BARB4 target) that intact BARB4 antibody (as represented by antibody produced by hybridoma DSMZ Deposit No. DSM ACC2876, deposited on Dec. 19, 2007, or heavy and light chain sequences set forth as SEQ ID NOs:1, 3, 5, 7; and 9, respectively) binds. In another embodiment, an antibody or functional fragment competes with BARB4 antibody, as represented by antibody produced by hybridoma DSMZ Deposit No. DSM ACC2876, deposited on Dec. 19, 2007, or heavy and light chain sequences set forth as SEQ ID NOs:1, 3, 5, 7; and 9, respectively, for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In another embodiment, an antibody or functional fragment competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In an additional embodiment, an antibody or functional fragment competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201).

Invention antibodies and functional fragments also include antibodies and functional fragments thereof that bind to cells or to an antigen (BARB4 target, such as a cell membrane bound isoform of a TAF 15 polypeptide) that BARB4 antibody (as represented by antibody produced by hybridoma DSMZ Deposit No. DSM ACC2876, or heavy and light chain sequences set forth as SEQ ID NOs:1, 3, 5, 7; and 9, respectively) binds. In one embodiment, an antibody or functional fragment binds to an adenocarcinoma cell or a squamous cell carcinoma (e.g., a stomach adenocarcinoma cell, a lung adenocarcinoma cell, a pancreas adenocarcinoma cell, a colon adenocarcinoma cell, a breast adenocarcinoma cell, or an esophagus squamous cell carcinoma) to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In another embodiment, an antibody or functional fragment binds to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In an additional embodiment, an antibody or functional fragment binds to a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201) that BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In a further embodiment, an antibody or functional fragment thereof binds to a BARB4 target, such as an antigen with complete (100%) or partial (e.g., 60%, 70%, 80%, 90%, 95% or more) sequence identity with TAF 15 polypeptide (e.g., a cell membrane bound TAF 15 polypeptide isoform, such as SEQ ID NO:11 or 12).

Invention antibodies and functional fragments further include a heavy or light chain variable region sequence with about 60% or more (e.g., 65%, 70%, 75%, 80%, 85%, 90%, 95%, etc.) identity to a heavy or light chain sequence variable regions as represented by antibody produced by hybridoma DSMZ Deposit No. DSM ACC2876, or as set forth in SEQ ID NOs:1, 3, 5, 7 or 9. In one embodiment, an antibody or subsequence thereof includes a sequence at least 60% or more identical to a heavy chain variable region sequence set forth as SEQ ID NO:1, 3, 5 or 7, or/and a sequence at least 60% or more (e.g., 65%, 70%, 75%, 80%, 85%, 90%, 95%, etc.) identical to a light chain variable region sequence set forth as SEQ ID NO:9. In another embodiment, an antibody or subsequence includes a sequence at least 80-85%, 85-90%, 90-95%, or 95-100% identical to one or more CDRs in heavy chain variable region sequence set forth as SEQ ID NO:1, 3, 5 or 7, or a sequence at least 80-85%, 85-90%, 90-95%, or 95-100% identical to one or more CDRs in a light chain variable region sequence set forth as SEQ ID NO:9.

Invention antibodies and functional fragments moreover include isolated and purified antibodies and functional fragments thereof that have one or more amino acid additions, deletions or substitutions of SEQ ID NOs:1, 3, 5, 7; or 9. In particular aspects, an antibody or functional fragment has a sequence at least 80-85%, 85-90%, 90-95%, or 95-100% identical to a heavy chain variable region sequence set forth as SEQ ID NO:1, 3, 5 or 7, or a sequence at least 80-85%, 85-90%, 90-95%, or 95-100% identical to a light chain variable region sequence set forth as SEQ ID NO:9. In further aspects, an antibody or functional fragment has a heavy or light chain sequence with 100% identity to one or more CDRs in a heavy or light chain variable region sequence as represented by antibody produced by hybridoma DSMZ Deposit No. DSM ACC2876, or set forth as SEQ ID NOs:1, 3, 5, 7; and 9, respectively, and has less than 100% identity to a region outside of the CDRs in a heavy or light chain variable region sequence as represented by antibody produced by hybridoma DSMZ Deposit No. DSM ACC2876, or set forth as SEQ ID NOs:1, 3, 5, 7; and 9, respectively.

Invention antibodies and functional fragments additionally include antibodies and functional fragments thereof that have a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to an antigen an antigen (BARB4 target, such as a cell membrane bound isoform of a TAF 15 polypeptide) or a cell (e.g., a neoplastic, cancer, tumor or metastatic cell, such as an adenocarcinoma cell or a squamous cell carcinoma, for example, a stomach adenocarcinoma cell, a lung adenocarcinoma cell, a pancreas adenocarcinoma cell, a colon adenocarcinoma cell, a breast adenocarcinoma cell, or an esophagus squamous cell carcinoma); or a human adenocarcinoma, squamous cell carcinoma, carcinoid carcinoma, invasive ductal carcinoma, gem cell carcinoma of stomach, lung, colon, pancreas, esophagus, prostate, breast or testis. In further embodiments, an antibody or functional fragment has a binding affinity within about 1-5000 fold of the binding affinity of BARB4 antibody (as represented by antibody produced by hybridoma DSMZ Deposit No. DSM ACC2876, or heavy and light chain sequences set forth as SEQ ID NOs:1, 3, 5, 7; and 9, respectively) for binding to a BARB4 target, such as a cell membrane bound isoform of a TAF 15 polypeptide, or for binding to a pancreas cancer cell (e.g., BXPC-3 cells), a colon cancer cell line (e.g., HT-29 cells), or a stomach cancer cell (e.g., 23132/87 cells). In still further embodiments, an antibody or functional fragment has a binding affinity within about KD 10⁻⁵ M to about KD 10⁻¹³ M for binding to a BARB4 target, such as a cell membrane bound isoform of a TAF 15 polypeptide (e.g., SEQ ID NO:11 or 12), or for binding to one or more cells or cell lines set forth herein (e.g., an adenocarcinoma cell, a squamous cell carcinoma, a stomach adenocarcinoma cell, a lung adenocarcinoma cell, a pancreas adenocarcinoma cell, a colon adenocarcinoma cell, a breast adenocarcinoma cell, an esophagus squamous cell carcinoma, etc.).

The invention additionally provides antibodies and functional fragments thereof that bind to a cell or cell line that expresses a cell membrane bound TAF 15 polypeptide isoform, as well as antibodies and functional fragments thereof that bind to a cell membrane bound TAF 15 polypeptide isoform antigen. Antibodies and functional fragments thereof moreover provided include those that compete with BARB4 antibody, as represented by DSMZ Deposit No. DSM ACC2859, or heavy and light chain sequences set forth as SEQ ID NOs:1 and 2, for binding to a TAF 15 polypeptide (e.g., SEQ ID NO:11 or 12), such as a cell membrane bound TAF 15 polypeptide isoform.

Antibodies of the invention include IgG, IgA, IgM, IgE and IgD. In various aspects, an IgG is an IgG1, IgG2, IgG3, or IgG4.

Invention antibodies and functional fragments moreover include those that inhibit or reduce proliferation, or stimulate or induce apoptosis, of a cell. In particular embodiments, antibodies and functional fragments inhibit or reduce proliferation, or stimulate or induce apoptosis of one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201).

Antigens (e.g., BARB4 target such as a cell membrane bound isoform of a TAF 15 polypeptide) of the invention include functional fragments and subsequences thereof. In one embodiment, a functional fragment of an antigen (e.g., BARB4 target such as a cell membrane bound isoform of a TAF 15 polypeptide) includes a carbohydrate moiety, such as an N- or O-linked moiety. In another embodiment, a functional fragment of an antigen (e.g., BARB4 target such as a cell membrane bound isoform of a TAF 15 polypeptide) includes an epitope to which BARB4 antibody (as represented by antibody produced by hybridoma DSMZ Deposit No. DSM ACC2876, or heavy and light chain sequences set forth as SEQ ID NOs:1, 3, 5, 7; and 9, respectively), binds.

Antibodies of the invention include functional fragments and subsequences thereof. In one embodiment, a functional fragment of an antibody, such as a BARB4 antibody (as represented by antibody produced by hybridoma DSMZ Deposit No. DSM ACC2876, or heavy and light chain sequences set forth as SEQ ID NOs:1, 3, 5, 7; and 9, respectively) competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to a cell or antigen (e.g., a BARB4 target, such as a cell membrane bound isoform of a TAF 15 polypeptide), or retains at least partial binding to a cell or antigen (e.g., a BARB4 target, such as a cell membrane bound isoform of a TAF 15 polypeptide) to which BARB4, as represented by antibody produced by hybridoma DSMZ Deposit No. DSM ACC2876, or heavy and light chain sequences set forth as SEQ ID NOs:1, 3, 5, 7; and 9, respectively, binds.

In particular aspects, an antibody functional fragment or a subsequence is an Fab, Fab′, F(ab′)₂, Fv, Fd, single-chain Fv (scFv), disulfide-linked Fvs (sdFv), V_{L}, V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc or (scFv)₂-Fc. In additional aspects, a functional fragment or a subsequence of a full length antibody (e.g., a heavy or light chain, or a heavy or light chain variable region), or an antigen (e.g., BARB4 target such as a cell membrane bound isoform of a TAF 15 polypeptide) has a length from about 20-30, 30-50, 50-100, 100-150, 150-200, 200-250, 250-300, 300-400, 400-500, amino acid residues.

The invention also provides antibodies, antigens (e.g., BARB4 target such as a cell membrane bound isoform of a TAF 15 polypeptide) and subsequences thereof that include a heterologous domain. In one embodiment, a heterologous domain includes a detectable label, tag or cytotoxic agent. In particular aspects, a detectable label or tag is an enzyme, enzyme substrate, ligand, receptor, radionuclide, a T7-, His-, myc-, HA- or FLAG-tag, electron-dense reagent, energy transfer molecule, paramagnetic label, fluorophore, chromophore, chemi-luminescent agent, or a bio-luminescent agent.

The invention moreover provides nucleic acid sequences that encode antigens (e.g., BARB4 target such as a cell membrane bound isoform of a TAF 15 polypeptide), antibodies and functional fragments thereof. In one embodiment, a nucleic acid sequence is at least 75-100% complementary or identical to a nucleic acid sequence that encodes a BARB4 target such as a cell membrane bound isoform of a TAF 15 polypeptide. In another embodiment, a nucleic acid sequence is at least 75-100% complementary or identical to a nucleic acid sequence that encodes a heavy or a light chain variable region sequence of BARB4 antibody, as represented by antibody produced by hybridoma DSMZ Deposit No. DSM ACC2876, or heavy and light chain sequences set forth as SEQ ID NOs:1, 3, 5, 7; and 9, respectively, or a subsequence thereof. In a further embodiment, a nucleic acid encodes a subsequence of a BARB4 target such as a cell membrane bound isoform of a TAF 15 polypeptide. In a still further embodiment, a nucleic acid encodes a subsequence of SEQ ID NOs:1, 3, 5, 7; and 9, respectively. In particular aspects, a nucleic acid sequence has a length from about 10-20, 20-30, 30-50, 50-100, 100-150, 150-200, 200-250, 250-300, 300-400, 400-500, or 500-1000 nucleotides. In additional aspects, a nucleic acid sequence specifically hybridizes to a nucleic acid that encodes a BARB4 target, such as a cell membrane bound isoform of a TAF 15 polypeptide (e.g., SEQ ID NO:11 or 12). In further aspects, a nucleic acid sequence specifically hybridizes to a nucleic acid that encodes SEQ ID NO:1, 3, 5 or 7, or a subsequence thereof, or specifically hybridizes to a nucleic acid sequence complementary to a nucleic acid that encodes SEQ ID NO:9, or a subsequence thereof. In further aspects, a nucleic acid is an antisense polynucleotide, a small interfering RNA, or a ribozyme nucleic acid that specifically hybridizes to a nucleic acid sequence, such as a nucleic acid encoding a BARB4 target, such as a cell membrane bound isoform of a TAF 15 polypeptide (e.g., SEQ ID NO:11 or 12), or a nucleic acid encoding SEQ ID NOs:1, 3, 5, 7 or 9. Antisense polynucleotides, small interfering RNA, and ribozyme polynucleotides can have a length from about 10-20, 20-30, 30-50, 50-100, 100-150, 150-200, 200-250, 250-300, 300-400, 400-500, 500-1000, 1000-2000 nucleotides, and be at least 90% complementary or identical to a nucleic acid sequence that encodes a BARB4 target, such as a cell membrane bound isoform of a TAF 15 polypeptide (e.g., SEQ ID NO:11 or 12), or that encodes SEQ ID NOs:1, 3, 5, 7 or 9, or any subsequence thereof. In still further aspects, nucleic acid sequence can include an expression control sequence or a vector (e.g., a viral, bacterial, fungal or mammalian vector).

The invention additionally provides isolated and purified cells as well as transformed host cells that express an antigen (BARB4 target), such as a cell membrane bound isoform of a TAF 15 polypeptide, or an antibody or subsequence thereof that includes a sequence at least 60% or more (e.g., 65%, 70%, 75%, 80%, 85%, 90%, 95%, etc.) identical to a heavy chain variable region sequence set forth as SEQ ID NO:1, 3, 5 or 7, or a sequence at least 60% or more (e.g., 65%, 70%, 75%, 80%, 85%, 90%, 95%, etc.) identical to a light chain variable region sequence of BARB4 antibody set forth as SEQ ID NO:9, or as represented by antibody produced by hybridoma DSMZ Deposit No. DSM ACC2876, or heavy and light chain sequences set forth as SEQ ID NOs:1, 3, 5, 7; and 9, respectively, and subsequences thereof. Such cells include eukaryotic and non-eukaryotic cells, which can stably or transiently express antigen (e.g., BARB4 target), antibody or subsequence thereof, or be stably or transiently transformed with the nucleic acid or vector that encodes antigen (e.g., BARB4 target), antibody or subsequence thereof.

The invention further provides kits. In various embodiments, a kit includes an antigen (e.g., BARB4 target), or an antibody or functional fragment thereof that competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to an antigen (e.g., BARB4 target) or to a cell (e.g., a neoplastic, cancer, tumor or metastatic cell). In particular aspects, a kit includes an antigen (e.g., BARB4 target), or an antibody or functional fragment thereof that competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In an additional embodiment, a kit includes an antibody or functional fragment thereof that competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to a pancreas cancer cell (e.g., BXPC-3 cells), a colon cancer cell (e.g., HT-29 cells) or a stomach cancer cell (e.g., 23132/87 cells).

Kits of the invention also include antigen (e.g., BARB4 target), or antibodies or functional fragments that bind to cells or an antigen (e.g., BARB4 target) that BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In one embodiment, a kit includes an antibody or functional fragment that binds to BARB4 target, such as a cell membrane bound isoform of a TAF 15 polypeptide. In another embodiment, a kit includes an antibody or functional fragment binds to an adenocarcinoma cell or a squamous cell carcinoma to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds, such as a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In a further embodiment, a kit includes an antibody or functional fragment binds to a human adenocarcinoma, squamous cell carcinoma, carcinoid carcinoma, ivasive ductal carcinoma, germ cell carcinoma of stomach, lung, colon, pancreas, esophagus, prostate, breast or testis to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In an additional embodiment, a kit includes an antibody or functional fragment that binds to a pancreas cancer cell (e.g., BXPC-3 cells), a colon cancer cell (e.g., HT-29 cells) or a stomach cancer cell (e.g., 23132/87 cells) that BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds.

Kits of the invention further include antigen (e.g., BARB4 target, such as a cell membrane bound isoform of a TAF 15 polypeptide, e.g., SEQ ID NO:11 or 12), antibodies and functional fragments that include a sequence, with 100% or less identity to a TAF 15 polypeptide sequence or a heavy or light chain variable region sequence. In one embodiment, a kit includes a sequence with about 60% or more (e.g., 65%, 70%, 75%, 80%, 85%, 90%, 95%, etc.) identity to a TAF 15 polypeptide sequence. In another embodiment, a kit includes a heavy or light chain sequence with about 60% or more (e.g., 65%, 70%, 75%, 80%, 85%, 90%, 95%, etc.) identity to light and heavy chain variable region sequences of BARB4 antibody, as represented by antibody produced by hybridoma DSMZ Deposit No. DSM ACC2876, or heavy and light chain sequences set forth as SEQ ID NOs:1, 3, 5, 7; and 9, respectively. In another embodiment, a kit includes an antibody or subsequence thereof with a sequence at least 60% or more (e.g., 65%, 70%, 75%, 80%, 85%, 90%, 95%, etc.) identical to a heavy chain variable region sequence set forth as SEQ ID NO:1, 3, 5 or 7, and to a sequence at least 60% or more (e.g., 65%, 70%, 75%, 80%, 85%, 90%, 95%, etc.) identical to a light chain variable region sequence set forth as SEQ ID NO:9. In further embodiments, a kit includes an antibody or subsequence with a sequence at least 80-85%, 85-90%, 90-95%, 95-100% identical to a TAF 15 polypeptide sequence, a sequence at least 80-85%, 85-90%, 90-95%, 95-100% identical to one or more CDRs in heavy chain variable region sequence set forth as SEQ ID NO:1, 3, 5 or 7, or a sequence at least 80-85%, 85-90%, 90-95%, 95-100% identical to one or more CDRs in a light chain variable region sequence set forth as SEQ ID NO:9.

In additional embodiments, a kit also includes an anti-cell proliferative or immune enhancing treatment or therapeutic agent, or an anti-neoplastic, anti-cancer or anti-tumor agent, or an article of manufacture (e.g., for delivering the antibody, anti-cell proliferative or immune enhancing treatment or therapy into a subject locally, regionally or systemically). In particular aspects, the instructions are for treating undesirable cell proliferation or a cell proliferative disorder (e.g., a neoplasia, tumor cancer or metastasis.

The invention yet additionally provides pharmaceutical compositions. In one embodiment, a composition includes an antigen (e.g., BARB4 target, such as a cell membrane bound isoform of a TAF 15 polypeptide), or a subsequence thereof, and a pharmaceutically acceptable carrier or excipient. In another embodiment, a composition includes an antibody or functional fragment and a pharmaceutically acceptable carrier or excipient. In a further embodiment, a composition includes an antibody that competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to a cell or antigen (e.g., BARB4 target, such as a cell membrane bound isoform of a TAF 15 polypeptide), or that binds to a cell or antigen (e.g., BARB4 target, such as a cell membrane bound isoform of a TAF 15 polypeptide) to which BARB4 antibody binds, or that includes a heavy or light chain variable region sequence with about 60% or more identity to a heavy or light chain sequence variable regions as set forth in SEQ ID NOs:1, 3, 5, 7; and 9, respectively, or a sequence at least 80-85%, 85-90%, 90-95%, 95-100% identical to one or more CDRs in a heavy chain or light chain variable region sequence set forth as SEQ ID NOs:1, 3, 5, 7; and 9, respectively, and a pharmaceutically acceptable carrier or excipient.

Antigens, antibodies, functional fragments and modified forms are useful for treating a subject in need of treatment. The invention therefore provides methods of using antigens, antibodies and functional fragments in treatment (e.g., therapeutic or prophylactic) of a subject having or at risk of having undesirable cell proliferation, such as a cell proliferative or hyperproliferative disorder. In one embodiment, a method includes administering an antigen (e.g., BARB4 target, such as a cell membrane bound isoform of a TAF 15 polypeptide), or a subsequence thereof, to a subject having or at risk of having undesirable cell proliferation (e.g., a cell proliferative disorder) an amount effective to inhibit or treat undesirable cell proliferation. In another embodiment, a method includes administering an antibody or functional fragment (e.g., a BARB4 antibody, as represented by antibody produced by hybridoma DSMZ Deposit No. DSM ACC2876, or heavy and light chain sequences set forth as SEQ ID NOs:1, 3, 5, 7; and 9, respectively) to a subject having or at risk of having undesirable cell proliferation (e.g., a cell proliferative disorder) an amount effective to inhibit or treat undesirable cell proliferation. In particular aspects, a cell proliferative disorder is a metastatic or non-metastatic, solid or liquid neoplasia, malignancy, tumor or cancer. In further aspects, undesirable cell proliferation (e.g., a cell proliferative disorder) affects or is present at least in part in brain, head or neck, breast, esophagus, mouth, nasopharynx, nose or sinuses, stomach, duodenum, ileum, jejunum, lung, liver, pancreas, kidney, adrenal gland, thyroid, bladder, colon, rectum, prostate, uterus, cervix, ovary, bone marrow, lymph, blood, bone, testes, skin or muscle, or hematopoetic system. In additional aspects, undesirable cell proliferation (e.g., a cell proliferative disorder) includes a neoplasia, tumor, cancer or metastasis that affects or is at least in part present in breast, lung, thyroid, head and neck, nasopharynx, nose or sinuses, brain, spine, adrenal gland, thyroid, lymph, gastrointestinal tract, mouth, esophagus, stomach, duodenum, ileum, jejunum, small intestine, colon, rectum, genito-urinary tract, uterus, ovary, cervix, bladder, testicle, penis, prostate, kidney, pancreas, adrenal gland, liver, bone, bone marrow, lymph, blood, muscle, skin or is hematopoetic. In further particular aspects, a neoplasia, tumor, cancer or metastasis is a sarcoma, carcinoma, adenocarcinoma, melanoma, myeloma, blastoma, glioma, lymphoma leukemia. In additional particular aspects, a neoplasia, tumor or cancer is a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, or uterine adenocarcinoma, or a metastasis thereof.

In a further embodiment, a method includes administering an antigen (e.g., BARB4 target, such as a cell membrane bound isoform of a TAF 15 polypeptide), or a subsequence thereof, to a subject having or at risk of having a metastasis an amount effective to reduce or inhibit spread or dissemination of a tumor, cancer or neoplasia to other sites, locations or regions within the subject. In an additional embodiment, a method includes administering an antibody or functional fragment (e.g., a BARB4 antibody, as represented by antibody produced by hybridoma DSMZ Deposit No. DSM ACC2876, or heavy and light chain sequences set forth as SEQ ID NOs:1, 3, 5, 7; and 9, respectively) to a subject having or at risk of having a metastasis an amount effective to reduce or inhibit spread or dissemination of a tumor, cancer or neoplasia to other sites, locations or regions within the subject.

In various aspects, a method reduces or inhibits metastasis of a primary tumor or cancer to one or more other sites, the formation or establishment of a metastasis at one or more other sites, thereby inhibiting or reducing tumor or cancer relapse or tumor or cancer progression. In further aspects, a method reduces or inhibits growth, proliferation, mobility or invasiveness of tumor or cancer cells that potentially or do develop metastases (e.g, disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established.

In further particular aspects, a neoplasia, tumor or cancer, or metastasis is progressively worsening, stable or is in remission. In still additional aspects, treatment results in alleviating or ameliorating one or more adverse physical symptoms associated with a cell proliferative disorder, or a neoplasia, tumor or cancer, or reduces or decreases neoplasia, tumor or cancer volume, inhibits or prevents an increase in neoplasia, tumor or cancer volume, inhibits neoplasia, tumor or cancer progression or worsening, stimulates neoplasia, tumor or cancer cell lysis or apoptosis, or inhibits, reduces or decreases neoplasia, tumor or cancer proliferation or metastasis, or prolongs or extends lifespan of the subject, or improves the quality of life of the subject.

Methods include administration to a subject locally, regionally, or systemically. Exemplary subjects (e.g., mammals such as humans) include candidates for, and those undergoing, or having undergone an anti-cell proliferative or anti-hyperproliferative disorder (e.g., anti-neoplastic, anti-tumor, anti-cancer or anti-metastasis) or immune-enhancing treatment or therapy.

The invention yet also provides combined methods for treating a disorder in a subject in need of treatment. In one embodiment, a method includes administering to a subject an antigen (e.g., BARB4 target, such as a cell membrane bound isoform of a TAF 15 polypeptide), or a subsequence thereof, and an anti-cell proliferative or immune-enhancing treatment or therapy to a subject (e.g., prior to, substantially contemporaneously with or following each other). In another embodiment, a method includes administering to a subject an antibody that competes with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding of to a cell, or binds to a cell to which BARB4 antibody, as represented by antibody produced by hybridoma DSMZ Deposit No. DSM ACC2876, or heavy and light chain sequences set forth as SEQ ID NOs:1 and 2, binds and an anti-cell proliferative or immune-enhancing treatment or therapy to a subject (e.g., prior to, substantially contemporaneously with or following each other). In various aspects, an anti-cell proliferative or immune-enhancing treatment or therapy includes surgical resection, radiotherapy, radiation therapy, chemotherapy, immunotherapy, hyperthermia, an alkylating agent, anti-metabolite, plant extract, plant alkaloid, nitrosourea, hormone, nucleoside or nucleotide analogue, a lymphocyte, plasma cell, macrophage, dendritic cell, NK cell or B-cell, an antibody, a cell growth factor, a cell survival factor, a cell differentiative factor, a cytokine or a chemokine.

Antibodies and functional fragments thereof are useful for detecting, screening for and identifying the presence of cells that bind to BARB4 antibody, as represented by antibody produced by hybridoma DSMZ Deposit No. DSM ACC2876, or heavy and light chain sequences set forth as SEQ ID NOs:1, 3, 5, 7; and 9, respectively, or antigen (e.g., TAF 15 polypeptide) that binds to BARB4 antibody, as represented by antibody produced by hybridoma DSMZ Deposit No. DSM ACC2876, or heavy and light chain sequences set forth as SEQ ID NOs:1, 3, 5, 7; and 9, respectively. The invention therefore provides methods for detecting or screening for cells and antigens (e.g., TAF 15 polypeptide) that bind to BARB4 antibody, as represented by antibody produced by hybridoma DSMZ Deposit No. DSM ACC2876, or heavy and light chain sequences set forth as SEQ ID NOs:1, 3, 5, 7; and 9, respectively, methods for identifying a subject that is amenable to treatment in accordance with the methods of the invention. In one embodiment, a method includes contacting a biological material or sample with an antibody or functional fragment under conditions allowing binding between antibody or functional fragment and cell or antigen that binds to BARB4 antibody, as represented by antibody produced by hybridoma DSMZ Deposit No. DSM ACC2876, or heavy and light chain sequences set forth as SEQ ID NOs:1, 3, 5, 7; and 9, respectively, and assaying for binding of the antibody or functional fragment to a cell or antigen that binds to BARB4 antibody, as represented by antibody produced by hybridoma DSMZ Deposit No. DSM ACC2876, or heavy and light chain sequences set forth as SEQ ID NOs:1, 3, 5, 7; and 9, respectively. The binding of the antibody or functional fragment to a cell or antigen that binds to BARB4 antibody, as represented by antibody produced by hybridoma DSMZ Deposit No. DSM ACC2876, or heavy and light chain sequences set forth as SEQ ID NOs:1, 3, 5, 7; and 9, respectively, indicates that the biological material contains the cell or antigen that binds to BARB4 antibody, as represented by antibody produced by hybridoma DSMZ Deposit No. DSM ACC2876, or heavy and light chain sequences set forth as SEQ ID NOs:1, 3, 5, 7; and 9, respectively. In one aspect, the biological material or sample is obtained from a mammalian (e.g., primate, such as a human) subject.

The invention moreoever provides methods for detecting or screening for an antigen (BARB4 target) having sequence identity to TAF 15 polypeptide, for example, a sequence identical to 10, 20, 30, 40, 50, 60, 70, 80, 90 or 100 or more (e.g., full length) contiguous amino acids set forth in SEQ ID NO:11 or 12), a cell membrane bound TAF 15 polypeptide isoform, or a TAF 15 polypeptide that includes a carbohydrate moiety. In one embodiment, a method includes contacting a biological material or sample with the antibody or functional fragment under conditions allowing binding of the antibody to an antigen having sequence identity to TAF 15 polypeptide; and assaying for binding of the antibody to the antigen having sequence identity to TAF 15 polypeptide. Binding of the antibody to the antigen detects the presence of the antigen having sequence identity to TAF 15 polypeptide.

The invention still moreoever provides methods for diagnosing a subject having or at increased risk of having undesirable cell proliferation or a cell proliferative disorder (e.g., neoplasia, tumor or cancer, or metastasis). In various embodiments, a method includes contacting a biological material or sample from a subject with an antibody or functional fragment thereof as set forth herein under conditions allowing binding of the antibody or functional fragment, and assaying for binding of the antibody to a cell or antigen (BARB4 target) having sequence identity to TAF 15 polypeptide, for example, a sequence identical to 10, 20, 30, 40, 50, 60, 70, 80, 90 or 100 or more (e.g., full length) contiguous amino acids set forth in SEQ ID NO:11 or 12), a cell membrane bound TAF 15 polypeptide isoform, or a TAF 15 polypeptide that includes a carbohydrate moiety. In particular aspects, the methods for diagnosing a subject identify those that have or are at increased risk of having undesirable cell proliferation or a cell proliferative disorder (e.g., neoplasia, tumor or cancer, or metastasis). For example, the presence of a TAF 15 polypeptide or cell surface expression of TAF 15 polypeptide can be indicative of increased risk of having undesirable cell proliferation or a cell proliferative disorder (e.g., neoplasia, tumor or cancer, or metastasis). In one aspect, the biological material or sample is obtained from a mammalian (e.g., primate, such as a human) subject. In additional aspects, the biological material or sample comprises a biopsy, such as a lung, pancreas, stomach, breast, esophageal, ovarian or uterine biopsy.

### DESCRIPTION OF DRAWINGS

FIGS. 1A-1F: BARB4 cell proliferation inhibitory activity of A) and B) pancreas cancer cells (BXPC-3); C) and D) stomach cancer cells (23132/87); and E) and F) colon cancer cells (HT-29), at 24 hours and 48 hours.

FIG. 2: BARB4 cell death activity of stomach cancer cells at the indicated time period.

FIG. 3: BARB4 malignant melanoma cancer cell (HTB-69) proliferation inhibitory activity.

FIG. 4: Immunofluorescence of BARB4 showing endocytosis of BARB4 antibody by pancreas carcinoma cells (BXPC-3) 30 minutes after binding.

FIG. 5: Reduced binding of BARB4 to BXPC-3 cancer cells treated with N-glycosidase, but not O-glycosidase.

FIGS. 6A-6B: A) BARB4 antibody binds to a membrane molecule with a relative molecular mass between 70 and 85 kDa (arrow); B) Coomassie stained gel of enriched and purified membrane extract indicating position of BARB4 target (arrow).

FIGS. 7A-7B: A) BARB4 target isolated from SDS-PAGE reduced with DTT, alkylated with iodine acetamid and digested by trypsin over night and measured by MALDI MS; B) Database comparison of peptide masses to human sequences in NCBI database.

FIG. 8A-8B: FACS analysis demonstrated that BARB4 antibody exhibits reduced binding to cells treated with siRNA against human TAF 15 mRNA, but not to untreated cells or cells treated with unrelated siRNA (negative control) after 48 h.

FIG. 9A-9C: BARB4 immunopreciptates TAF15, as confirmed by Western blotting with two different TAF15 antibodies, A) anti-TAFII68; and C) anti-TAF15 (ARP30111_T100, AVIVA).

### DETAILED DESCRIPTION

The invention is based, at least in part, on a glycoprotein antigen, referred to as BARB4 target. BARB4 target has an apparent molecular weight in a range of about 70-85 kilodaltons (KDa) as determined by denaturing (SDS) gel electrophoresis. A non-limiting exemplary feature of BARB4 target is expression on cell surface. Another non-limiting exemplary feature of BARB4 target is linkage of at least one nitrogen (N)- or oxygen (O)-linked carbohydrate moiety. A further non-limiting exemplary feature of BARB4 target is that an antibody denoted BARB34, as represented by antibody produced by hybridoma DSMZ Deposit No. DSM ACC2876, or heavy and light chain sequences set forth as SEQ ID NOs:1, 3, 5, 7; and 9, respectively, specifically binds to an epitope present on BARB4 target.

Sequence analysis of BARB4 target revealed at least partial sequence identity with TATA-binding protein-associated factor 15 (TAF 15 polypeptide), also known and referred to as TAF(II)68, TAF2N and RNA-binding protein 56 (RBP56). Human TATA-binding protein-associated factor 15 (TAF 15 polypeptide) sequence isoforms are as set forth in SEQ ID NO:11 and 12. Sequences of BARB4 target that appear identical to TATA-binding protein-associated factor 15 (TAF 15 polypeptide) sequence are underlined as follows:

| | |
|---|---|
| 1 | msdsgsygqsggeqqsystygnpgsqgygqasqsysgygqttdssygqnysgyssygqsq |
| 61 | sgysqsyggyenqkqssysqqpynnqgqqqnmessgsqggrapsydpdy gqqdsydqqs |
| 121 | gydqhqgsydeqsnydqqhdsysqnqqsyhsqranyshhtqddrrdvsrygednrgyggs |
| 181 | qgggrgrggydkdgrgpmtgssggdrggfknfgghrdygprtdadsesdnsdnntifvqg |
| 241 | lgegvstdqvgeffkqigiiktnkktgkpminlytdkdtgkpkgeatvsfddppsakaai |
| 301 | dwfdgkefhgniikvsfatrrpefmrgggsgggrrgrggyrgrggfqgrggdpksgdwvc |
| 361 | pnpscgnmnfarrnscnqcneprpedsrpsggdfrgrgyggergyrgrggrggdrggygg |
| 421 | drsgggyggdrssgggysgdrsgggyggdrsgggyggdrgggyggdrgggyggdrgggyg |
| 481 | gdrggyggdrgggyggdrggyggdrggyggdrggyggdrggyggdrsrggyggdrgggsg |
| 541 | yggdrsggyggdrsgggyggdrgggyggdrggyggkmggrndyrndqrnrpy |

Thus, another non-limiting exemplary feature of BARB4 target is complete or at least partial sequence homology/identity with human TATA-binding protein-associated factor 15 (TAF 15 polypeptide) sequence as set forth in SEQ ID NO:11 or 12.

TATA-binding protein-associated factor 15 (TAF 15 polypeptide) has not been characterized to be expressed on the cell (extracellular) surface. Thus, still another non-limiting exemplary feature of BARB4 target is its apparent identity as a cell membrane bound isoform of a TAF 15 polypeptide.

In accordance with the invention, there are provided isolated and purified antigens, denoted BARB4 target and subsequences of BARB4 target. In one embodiment, a BARB4 target has sequence identity with TATA-binding protein-associated factor 15 (TAF 15 polypeptide) as set forth in SEQ ID NO:11 or 12, e.g., complete (100%) or partial (e.g., 60%, 70%, 80%, 90%, 95% or more) identity to TAF 15 polypeptide. In another embodiment, a BARB4 target or subsequence thereof is a cell membrane bound isoform of a TAF 15 polypeptide, with sequence identity to TATA-binding protein-associated factor 15 (TAF 15 polypeptide) set forth in SEQ ID NO:11 or 12, complete (100%) or partial (e.g., 60%, 70%, 80%, 90%, 95% or more) identity. In a further embodiment, a BARB4 target or subsequence thereof has a sequence that is completely (100%) or partially (e.g., 60%, 70%, 80%, 90%, 95% or more) identical to 10, 20, 30, 40, 50, 60, 70, 80, 90 or 100 or more contiguous amino acids in TAF 15 polypeptide (e.g., SEQ ID NO:11 or 12). In further embodiments, a BARB4 target or subsequence thereof includes a carbohydrate moiety (e.g., an N-linked carbohydrate moiety or an O-linked carbohydrate moiety), or is expressed by a tumor or cancer cell (e.g., a pancreas cancer or tumor cell, a colon cancer or tumor cell, or a stomach cancer or tumor cell, such as BXPC-3 cells, HT-29 cells or 23132/87 cells). In additional embodiments, a BARB4 target or subsequence thereof is an antigen to which a BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds, or has a molecular weight of about 70-85 KDa, as determined by sodium dodecyl sulfate polyacrylamide gel electrophoresis (SDS-PAGE). BARB4 target embodiments include a fusion protein resulting from a chromosomal translocation of or into a gene encoding a TAF 15 polypeptide. BARB4 target embodiments also exclude a fusion protein resulting from a chromosomal translocation of or into a gene encoding the TAF 15 polypeptide (e.g., BARB4 target is not a fusion protein resulting from a chromosomal translocation of a TAF 15 fused to a nuclear receptor, NOR1).

As used herein the term “isoform” refers to a protein that is a variant as compared to a reference protein. The variation can be in one or more amino acid residues, such as small differences in amino acid sequence as compared to a reference protein. The variation can be an insertion, deletion or substitution of one or more amino acid residues as compared to a reference protein. Isoforms of a TATA-binding protein-associated factor 15 (TAF 15 polypeptide) are set forth in SEQ ID NO:11 and 12, and additional isoforms can have partial (e.g., 60%, 70%, 80%, 90%, 95% or more) sequence identity to a reference TAF 15 polypeptide (e.g., SEQ ID NO:3 or 4). An isoform may have the same polypeptide sequence but may differ in terms of postranslational processing, such as phosphorylation, glycosylation, amidation, etc. Thus, an isoform of a TATA-binding protein-associated factor 15 (TAF 15 polypeptide) can have complete (e.g., 100%) sequence identity to SEQ ID NO:11 or 12, but can differ in terms of posttranslational processing. An isoform may be encoded by the same or a different gene. An isoform may also have a different function, or different cellular localization compared to a reference TAF 15 polypeptide. For example, TATA-binding protein-associated factor 15 (TAF 15 polypeptide) is a transcription factor that is typically present in the nucleus. However, BARB4 target, which has at least partial sequence identity with TAF 15 polypeptide set forth in SEQ ID NO:11 and 12 is present on the cell surface of certain tumor, cancer and neoplastic cells. Because BARB4 target has a different cellular location, BARB4 target can be referred to as a TATA-binding protein-associated factor 15 (TAF 15 polypeptide) isoform.

As used herein the term “glycoprotein” refers to a protein, polypeptide or peptide that has at least one sugar moiety covalently linked to an amino acid comprising the protein. A “carbohydrate moiety” refers to two or more sugar residues, e.g., mono-, di-, tri-saccharides, etc. The terms oligosaccharide and polysaccharide are synonymous with the term carbohydrate. A “carbohydrate moiety” can comprise all or a part of an epitope present on BARB4 target to which an antibody binds (e.g., BARB4 antibody, as represented by antibody produced by hybridoma DSMZ Deposit No. DSM ACC2876, deposited on Dec. 19, 2007, or heavy and light chain sequences set forth as SEQ ID NOs:1, 3, 5, 7; and 9, respectively).

Sugars, carbohydrates, oligosaccharides and polysaccharides are typically linked to amino acid residues by a glycosidic bond. For eukaryotes, a series of sugar additions and removals occur post-translationally to form the carbohydrate moiety of the glycoprotein. Exemplary sugars include one or more of galactose, acetylgalactose, mannose, fucose, glucose, acetylglucose, sialic acid, N-acetylgalactosamine, N-acetylglucosamine and neuramic acids. In a particular aspect, BARB4 target has have one or more of such particular sugars attached via an N- or O-linkage to a serine, threonine or asparagine residue, for example.

Contact of BARB4 target with a glycosidase enzyme can reduce the apparent molecular weight of BARB4 target due to removal of one or more sugar residues comprising the carbohydrate moiety. Thus, in one aspect, contact of BARB4 target with an N-glycosidase enzyme reduces the apparent molecular weight of BARB4 target from 70-85 KDa.

Glycosidases capable of removing one or more sugars of a carbohydrate moiety, or the entire carbohydrate structure, include O-glycosidases, which typically cleave sugars that comprise carbohydrate moieties linked to the oxygen (O) of serine or threonine residues, and N-glycosidases, which typically cleave sugars that comprise carbohydrate moieties linked to the nitrogen (N) of asparagine residues. Particular examples of such glycosidases are O-glycosidase, N-glycosidase F, endoglycosidase H (endo H), neuraminidase and fucosidases. O-glycosidase cleaves serine- or threonine-linked oligosaccharide. N-glycosidase F cleaves asparagine bound N-glycans when the oligosaccharide has a minimum length of the chitobiose core unit. Endo H is a glycosidase that cleaves within the chitobiose core of high mannose and some hybrid oligosaccharides from N-linked glycoproteins. Neuraminidase removes terminal acylneuraminic residues. Fucosidases remove fucose, for example, from lactose and complex carbohydrates. Such glycosidases typically have at least some specificity in terms of the sugar linkages cleaved and whether the carbohydrate moieties are O- or N-linked and can therefore be used to characterize the composition and structure of the BARB4 target carbohydrate moiety(ies).

In embodiments in which antibody denoted BARB4 specifically binds to BARB4 target, the BARB4 antibody binds to at least a portion of the BARB4 target (epitope) comprising a carbohydrate moiety (e.g., an N- or O-linked carbohydrate moiety). Contact of BARB4 target with an N-glycosidase enzyme reduced binding of BARB4 antibody, as represented by antibody produced by hybridoma DSMZ Deposit No. DSM ACC2876, or heavy and light chain sequences set forth as SEQ ID NOs:1, 3, 5, 7; and 9, respectively, to the glycoprotein, presumably due to removal of one, several or all sugar(s) including a BARB4 binding epitope. Accordingly, BARB4 binding to BARB4 target may require or be mediated by, at least in part, one or more sugars comprising an N-linked carbohydrate moiety of a BARB4 target epitope.

Thus, in another aspect, a BARB4 target treated with an N-glycosidase enzyme reduces the apparent binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for BARB4 target.

The invention is also based, at least in part, on antibodies that bind to an antigen (BARB4 target, such as a cell membrane bound isoform of a TAF 15 polypeptide) expressed by various neoplastic, cancer, tumor and metastatic cells. A non-limiting exemplary antibody is designated BARB4, as represented by antibody produced by hybridoma DSMZ Deposit No. DSM ACC2876, or heavy and light chain sequences set forth as SEQ ID NOs:1, 3, 5, 7; and 9, respectively. BARB4, as represented by antibody produced by hybridoma DSMZ Deposit No. DSM ACC2876, or heavy and light chain sequences set forth as SEQ ID NOs:1, 3, 5, 7; and 9, respectively, is a human IgG antibody that specifically binds to various neoplastic, cancer, tumor and metastatic cells. BARB4 is able to inhibit or reduce proliferation of various neoplastic, cancer, tumor and metastatic cells. BARB4 is also able to stimulate or induce apoptosis of various neoplastic, cancer, tumor and metastatic cells.

Antibodies of the invention include polyclonal and monoclonal antibodies. Antibodies are proteins which include amino acids, or “residues,” covalently linked by an amide bond or equivalent. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies of the invention can belong to any antibody class, IgM, IgG, IgE, IgA, IgD, or subclass. Exemplary subclasses for IgG are IgG₁, IgG₂, IgG₃ and IgG₄.

Antibodies of the invention can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains. The primary difference between kappa and lambda light chains is in the sequences of the constant region.

The amino acid sequences of BARB4, heavy and light chain variable region sequences, as represented by SEQ ID NOs:1, 3, 5 and 7; and 9, respectively, are shown.

Amino acid sequence of BARB4 heavy chain (VH; SEQ ID NO:1):

QVQLVESGGGVVQPGRSLRLSCAASGFRFTTHGMHWVRQAPGKGLEWVAV
ISYNGRNKYYADYVNGRFTISRDDSRDTVFLQMNSLRPEDTAMYYCAKVR
GDGYGDYGYFDYWGHGTLVSVSS

Amino acid sequence of BARB4 heavy chain (4.1 VH; SEQ ID NO:3):

EVQLVESGGGVVQPGRSLRLSCAASGFRFTTHGMHWVRQAPGKGLEWVAV
ISYNGRNKYYADYVNGRFTISRDDSRDTVFLQMNSLRPEDTAMYYCAKVR
GDGYGDYGYFDYWGHGTLVSVSS

Amino acid sequence of BARB4 heavy chain (4.2 VH; SEQ ID NO:5):

EVQLVESGGGLVQPGGSLRLSCAASGFRFTTHGMHWVRQAPGKGLEWVAV
ISYNGRNKYYADYVNGRFTISRDDSRDTVFLQMNSLRPEDTAMYYCAKVR
GDGYGDYGYFDYWGHGTLVSVSS

Amino acid sequence of BARB4 heavy chain (4.3 VH; SEQ ID NO:7):

QVQLVESGGGVVQPGRSLRLSCAASGFRFTTHGMHWVRQAPGKGLEWVAV
ISYNGRNKYYADYVNGRFTISRDDSRDTVFLQMNSLRPEDTAMYYCAKVR
GDGYGDYGYFDYWGHGTLVTVSS

Amino Acid Sequence of BARB4 light chain (VL; SEQ ID NO:9):

QSALTQPRSVSGSPGQSVTISCTGTYNYVSWYQQHPGKAPKLMIFDVSRR
SSGVPDRFSGSKSGNTASLTISGLQAEDEADYYCCSYGGTYLYVFGTGTT
VTVLGQ.

Predicted CDRs, of which there are three in each of heavy and light chain sequence set forth as SEQ ID NOs:1 and 2, are conveniently denoted herein as LC-CDR1, LC-CDR2 and LC-CDR3; and HC-CDR1, HC-CDR2 and HC-CDR3. The CDR sequences of each of SEQ ID NOs:1 and 2 were determined by alignments with homologous germline sequences. Various sequence databases such as MRC (VBASE, MRC Centre for Protein Engineering) and IMGT (International ImMunoGeneTics Information System) databases can be used to determine CDR positions. In particular, to determine the positions of the CDRs MRC (VBASE, MRC Centre for Protein Engineering) sequence database was used. Typically, the IMGT database gives shorter CDR regions for lambda light chain.

Based upon the MRC database, BARB4 Heavy Chain CDR1, GFRFTTHGMH; CDR2, VISYNGRNKYYA; and CDR3, VRGDGYGDYGYF. Based upon the MRC database, BARB4 Light Chain CDR1, TGTYNYVS; CDR2, DVSRRSS; and CDR3, CSYGGTYLY. The location of additional regions, such as D- and J-regions are also known to the skilled artisan.

In accordance with the invention, there are provided isolated and purified antibodies and functional (e.g., cell or antigen binding) fragments structurally and/or functionally related to BARB4, as represented by antibody produced by hybridoma DSMZ Deposit No. DSM ACC2876, or heavy and light chain variable region sequences set forth as SEQ ID NOs:1, 3, 5, 7; and 9, respectively. In various embodiments, antibodies and functional fragments compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to a cell or antigen (e.g., BARB4 target, such as a cell membrane bound isoform of a TAF 15 polypeptide, or an epitope thereon). In additional embodiments, antibodies and functional fragments compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to an adenocarcinoma cell or a squamous cell carcinoma. In further embodiments, antibodies and functional fragments compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In yet additional embodiments, antibodies and functional fragments compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to a human adenocarcinoma, human squamous cell carcinoma, human carcinoid carcinoma, human invasive ductal carcinoma, or a human germ cell carcinoma of any of stomach, lung, colon, pancreas, esophagus, prostate, breast or testis. In still further embodiments, antibodies and functional fragments compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201). In particular aspects, antibodies and functional fragments competitively inhibit binding of BARB4 antibody, as represented by antibody produced by hybridoma DSMZ Deposit No. DSM ACC2876, or heavy and light chain variable region sequences set forth a SEQ ID NOs:1, 3, 5, 7; and 9, respectively, to a cell or antigen (e.g., BARB4 target, such as a cell membrane bound isoform of a TAF 15 polypeptide) by at least 30%, 40%, 50%, 60%, 70%, 80%, 90% or more.

In accordance with the invention, there are also provided antibodies and functional fragments that bind to a cell or antigen (e.g., BARB4 target, such as a cell membrane bound isoform of a TAF 15 polypeptide) or epitope that BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds. In one embodiment, an isolated or purified antibody or functional fragment thereof binds to a cell or antigen (e.g., BARB4 target, such as a cell membrane bound isoform of a TAF 15 polypeptide) or epitope that BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds. In another embodiment, an isolated or purified antibody or functional fragment thereof binds to a BARB4 target, such as an antigen with complete (100%) or partial (e.g., 60%, 70%, 80%, 90%, 95% or more) identity sequence identity to TATA-binding protein-associated factor 15 set forth in SEQ ID NO:11 or 12. In particular aspects, the antibody or functional fragment thereof binds to a cell or antigen (e.g., BARB4 target, such as a cell membrane bound isoform of a TAF 15 polypeptide) or epitope present on an adenocarcinoma cell or a squamous cell carcinoma to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds. In additional particular aspects, the antibody or functional fragment thereof binds to a cell or antigen (e.g., BARB4 target, such as a cell membrane bound isoform of a TAF 15 polypeptide) or epitope present on one or more of a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds. In further particular aspects, the antibody or functional fragment thereof binds to a cell or antigen (e.g., BARB4 target, such as a cell membrane bound isoform of a TAF 15 polypeptide) or epitope present on a human adenocarcinoma, squamous cell carcinoma, carcinoid carcinoma, invasive ductal carcinoma, germ cell carcinoma of stomach, lung, colon, pancreas, esophagus, prostate, breast or testis to which intact BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. In still further particular aspects, the antibody or functional fragment thereof binds to a cell or antigen or epitope present on a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201) that intact BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds.

Cells (e.g., neoplastic, cancer, tumor or metastatic cells), cell lines (e.g., neoplastic, cancer, tumor or metastatic cell lines) or antigen to which antibodies bind include cells and cell lines that express a cell membrane isoform of TAF15 polypeptide, a TAF15 polypeptide that includes a carbohydrate moiety, and an antigen comprising a cell membrane isoform of TAF15 polypeptide. The invention therefore further provides antibodies and functional fragments thereof that bind to a cell or cell line that expresses a cell membrane bound TAF15 polypeptide isoform, a TAF15 polypeptide that includes a carbohydrate moiety, as well as antibodies and functional fragments thereof that bind to a cell membrane bound TAF15 polypeptide isoform antigen. Antibodies and functional fragments thereof moreover provided include those that compete with BARB4 antibody, as represented by DSMZ Deposit No. DSM ACC2876, or heavy and light chain variable region sequences set forth as SEQ ID NOs:1, 3, 5, 7; and 9, respectively, for binding to a TAF15 polypeptide sequence (e.g., a cell membrane bound TAF15 polypeptide, a TAF15 polypeptide that includes a carbohydrate moiety, or SEQ ID NO:11 or 12).

The term “bind,” or “binding,” when used in reference to an antibody or functional fragment, means that the antibody or functional fragment interacts at the molecular level with a corresponding epitope (antigenic determinant) present on a cell or an antigen. Epitopes of antigens that comprise amino acids typically include relatively short sequences, e.g. about five to 15 amino acids in length. Epitopes can be contiguous or non-contiguous. A non-contiguous amino acid sequence epitope forms due to protein folding. Techniques for identifying epitopes are known to the skilled artisan and include screening overlapping oligopeptides for binding to antibody (for example, U.S. Pat. No. 4,708,871), phage display peptide library kits, which are commercially available for epitope mapping (New England BioLabs). Epitopes may also be identified by inference when epitope length peptide sequences are used to immunize animals from which antibodies that bind to the peptide sequence are obtained and can be predicted using computer programs, such as BEPITOPE (Odorico et al., J. Mol. Recognit. 16:20 (2003)).

The invention further provides antibodies and functional fragments that inhibit, decrease or reduce cell growth or proliferation, or stimulate or induce cell death, lysis or apoptosis. In particular embodiments, binding of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, to a neoplastic, tumor or cancer, or metastasis cell inhibits, decreases or reduces cell growth or proliferation, or stimulates or induces cell death, lysis or apoptosis. In another embodiment, binding of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, to BXPC-3 or 23132/87 cells inhibits, decreases or reduces cell growth or proliferation, or stimulates or induces cell death, lysis or apoptosis.

The invention moreover provides of antibodies and functional fragments that are structurally and/or functionally related to BARB4 antibody, as represented by antibody produced by hybridoma DSMZ Deposit No. DSM ACC2876; or heavy and light chain variable region sequences set forth as SEQ ID NOs:1, 3, 5, 7; and 9, respectively, in which include the heavy or light chain variable region sequence exhibits a degree of identity to SEQ ID NOs:1, 3, 5, 7 or 9, respectively, or exhibits identity to a sequence within SEQ ID NOs:1, 3, 5, 7 or 9 (e.g., one or more CDRs). Antibodies and functional fragments of the invention therefore include those with at least partial sequence identity to BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively.

In particular embodiments, antibodies and functional fragments include a heavy or a light chain variable region sequence with about 60% or more identity to a heavy or light chain sequence variable region of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, or a sequence within SEQ ID NOs:1, 3, 5, 7 or 9 (e.g., one or more CDRs). In other particular embodiments, antibodies or functional fragments include a heavy or a light chain with at least 65%, 70%, 75%, 80%, 85%, 90%, 95%, or more identity to a heavy chain variable region sequence of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, or a sequence within SEQ ID NOs:1, 3, 5, 7 or 9 (e.g., one or more CDRs). In additional particular embodiments, antibodies or functional fragments include a heavy or a light chain variable region sequence with at least 80-85%, 85-90%, 90-95%, 95-100% identity to one or more CDRs in BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. In a particular aspect, an antibody or a functional fragment thereof includes a heavy or a light chain variable region sequence with 95-100% identity to one, two or three CDRs in each heavy or light chain variable region sequences in BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively.

In terms of percent identity of antigen (e.g., BARB4 target, such as a cell membrane bound isoform of a TAF 15 polypeptide), antibodies and functional fragments, identity can be as little as 60%, or can be more (e.g., 65%, 70%, 75%, 80%, 85%, 90%, 95%, etc.) to a reference sequence. The percent identity can extend over the entire sequence length of BARB4 target (e.g., a cell membrane bound isoform of a TAF 15 polypeptide), or BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, or a contiguous region or area within BARB4 target (e.g., a cell membrane bound isoform of a TAF 15 polypeptide) or BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids. In yet particular antibody or subsequence aspects, the length of the sequence sharing the percent identity is equal to the length of any CDR of a variable region sequence, or a region outside the CDRs but within the variable region of a heavy or light chain sequence, such as BARB4 antibody represented by DSMZ Deposit No. DSM ACC2876, or heavy and light chain variable region sequences set forth as SEQ ID NOs:1, 3, 5, 7; and 9, respectively.

The term “identity” and grammatical variations thereof, mean that two or more referenced entities are the same. Thus, where two antigen or antibody sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region. Exemplary antigens, antibodies and functional fragments have an amino acid sequence identity of 50%, 60%, 70%, 75%, 80%, 85%, 90%, 95%, 96%, 97%, 98%, or more to a reference BARB4 target (e.g., a cell membrane bound isoform of a TAF 15 polypeptide), for example, TAF 15 polypeptide set forth as SEQ ID NO:11 or 12, or to a BARB4 antibody or functional fragment thereof, as represented by antibody produced by hybridoma DSMZ Deposit No. DSM ACC2876, or heavy and light chain variable region sequences set forth as SEQ ID NOs:1, 3, 5, 7; and 9, respectively.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function) of the reference molecule, or relevant/corresponding region or portion of the reference molecule to which it shares homology. An antigen, antibody or functional fragment with substantial homology has or is predicted to have at least partial activity or function as the reference antibody. For example, in various embodiments for an antigen, a BARB4 target (e.g., a cell membrane bound isoform of a TAF 15 polypeptide, SEQ ID NO:11 or 12) or subsequence thereof retains an ability to be expressed on a cell membrane, has a carbohydrate moiety, or binds to a BARB4 antibody. In a particular embodiment for antibodies, a BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, with one or more modifications (e.g., substitutions, deletions or additions of SEQ ID NOs:1, 3, 5, 7 or 9) retain the ability to at least partially compete for binding of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, to a cell or antigen, or at least retains partial binding to a cell or antigen to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds is considered to have substantial homology to BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol. Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Antigens, antibodies and functional fragments of the invention include those that retain at least one or more partial activities or functions of antigen (e.g., BARB4 target, such as a cell membrane bound isoform of a TAF 15 polypeptide), or BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. As disclosed herein, the antigen to which BARB4 antibody, as represented by antibody produced by hybridoma DSMZ Deposit No. DSM ACC2876; or heavy and light chain sequences set forth as SEQ ID NOs:1, 3, 5, 7; and 9, respectively, binds has sequence identity with TAF 15 polypeptide set forth as SEQ ID NO:11 or 12. In various forms, BARB4 target is an apparent cell membrane bound isoform of a TAF 15 polypeptide, contains a carbohydrate moiety, and is expressed by various malignant and non-malignant, neoplastic, tumor and cancer cells. Thus, in various embodiments, a BARB4 target or subsequence thereof retains one or more of, sequence identity with TAF 15 polypeptide set forth as SEQ ID NO:11 or 12, an ability to be expressed on a cell membrane, has a carbohydrate moiety, or binds to a BARB4 antibody.

As also disclosed herein, BARB4, as represented by antibody produced by hybridoma DSMZ Deposit No. DSM ACC2876, or heavy and light chain variable region sequences set forth as SEQ ID NOs:1, 3, 5, 7; and 9, light and heavy chain respectively, binds to various malignant and non-malignant, neoplastic, tumor and cancer cells. Non-limiting examples of cells that bind to BARB4, as represented by antibody produced by hybridoma DSMZ Deposit No. DSM ACC2876, or heavy and light chain variable region sequences set forth as SEQ ID NOs:1, 3, 5, 7; and 9, respectively, light and heavy chain, and therefore express a target antigen of BARB4 include a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201). Thus, in various embodiments, an antibody or functional fragment binds to one or more cells, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201).

Antibodies and functional fragments that bind to a cell or antigen to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds can have greater or less relative binding affinity for a cell or an antigen (e.g., BARB4 target, such as a cell membrane bound isoform of a TAF 15 polypeptide) than BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. Additional antibodies and functional fragments of the invention therefore include those that have greater than, about the same or less than the binding affinity of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively light and heavy chain, for binding to a cell or antigen (e.g., BARB4 target, such as a cell membrane bound isoform of a TAF 15 polypeptide). For example, an antibody or functional fragment of the invention may have an affinity greater or less than 2-5, 5-10, 10-100, 100-1000 or 1000-10,000-fold affinity, or any numerical value or range within or encompassing such values, than BARB4 antibody, as represented by antibody produced by hybridoma DSMZ Deposit No. DSM ACC2876, or heavy and light chain sequences set forth as SEQ ID NOs:1, 3, 5, 7; and 9, respectively. In various embodiments, an antibody or a functional thereof has a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to an antigen (e.g., BARB4 target, such as a cell membrane bound isoform of a TAF 15 polypeptide), or for binding to a neoplastic, cancer, tumor or metastatic cell. In other embodiments, an antibody or a functional thereof has a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to an antigen (e.g., BARB4 target, such as a cell membrane bound isoform of a TAF 15 polypeptide), or for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In further embodiments, an antibody or a functional thereof has a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to antigen (e.g., BARB4 target, such as a cell membrane bound isoform of a TAF 15 polypeptide), or for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In additional embodiments, an antibody or a functional thereof has a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to antigen (e.g., BARB4 target, such as a cell membrane bound isoform of a TAF 15 polypeptide), or for binding to pancreas cancer BXPC-3 cells, cancer cells HT-29 (ATCC Deposit No. HTB-38) or stomach cancer 23132/87 cells. In the foregoing embodiments binding affinity can be 1-5000 fold greater or less than the binding affinity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding to antigen (e.g., BARB4 target, such as a cell membrane bound isoform of a TAF 15 polypeptide) or cells.

Antibodies and functional fragments include those having greater affinity for particular antigens than others. In particular embodiments, an antibody has a binding affinity for TAF 15 polypeptide expressed by a tumor or cancer cell greater than the binding affinity for TAF 15 polypeptide expressed in a non-tumor or non-cancer cell; an antibody has a binding affinity for a cell membrane bound TAF 15 polypeptide isoform greater than the binding affinity for TAF 15 polypeptide that is not cell membrane bound or is intracellular; and an antibody has a binding affinity for TAF 15 polypeptide that includes a carbohydrate moiety greater than the binding affinity for a TAF 15 polypeptide that lacks a carbohydrate moiety (e.g., an N-linked carbohydrate or an O-linked carbohydrate).

Binding affinity can be determined by association (Kₐ) and dissociation (K_{d}) rate. Equilibrium affinity constant, K, is the ratio of Kₐ/K_{d}. Association (Kₐ) and dissociation (K_{d}) rates can be measured using surface plasmon resonance (SPR) (Rich and Myszka, Curr. Opin. Biotechnol. 11:54 (2000); Englebienne, Analyst. 123:1599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)).

Additional specific non-limiting antibodies and functional fragments have binding affinity for a cell or antigen (e.g., BARB4 target, such as a cell membrane bound isoform of a TAF 15 polypeptide) to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, within about K_{d} 10⁻² M to about K_{d} 10⁻¹⁵ M, or within about K_{d} 10⁻⁵ M to about K_{d} 10⁻¹² M. In particular embodiments, binding affinity for is less than 5×10⁻² M, 10⁻² M, 5×10⁻³ M, 10⁻³ M 5×10⁻⁴ M, 10⁻⁴ M 5×10⁻⁵ M, 10⁻⁵ M 5×10⁻⁶ M, 10⁻⁶ M 5×10⁻⁷ M, 10⁻⁷ M 5×10⁻⁸ M, 10⁻⁸ M 5×10⁻⁹ M, 10⁻⁹ M 5×10⁻¹⁰ M, 10⁻¹⁰ M 5×10⁻¹¹ M, 10⁻¹⁵ M 5×10⁻¹² M, 10⁻¹² M 5×10⁻¹³ M, 10⁻¹³ M 5×10⁻¹⁴ M, 10⁻¹⁴ M 5×10⁻⁵ M, and 10⁻¹⁵ M. In additional particular embodiments, an antibody or functional fragment has a binding affinity within about K_{d} 10⁻⁵ M to about K_{d} 10⁻¹³ M for binding to antigen (e.g., BARB4 target, such as a cell membrane bound isoform of a TAF 15 polypeptide), or to a neoplastic, cancer, tumor or metastatic cell. In further particular embodiments, an antibody or functional fragment has a binding affinity within about K_{d} 10⁻⁵ M to about K_{d} 10⁻¹³ M for binding to antigen (e.g., BARB4 target, such as a cell membrane bound isoform of a TAF 15 polypeptide), or for binding to an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma. In other particular embodiments, an antibody or functional fragment has a binding affinity within about K_{d} 10⁻⁵ M to about K_{d} 10⁻¹³ M for binding to antigen (e.g., BARB4 target, such as a cell membrane bound isoform of a TAF 15 polypeptide), or for binding to a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis. In still further particular embodiments, an antibody or functional fragment has a binding affinity within about K_{d} 10⁻⁵ M to about K_{d} 10⁻¹³ M for binding to antigen (e.g., BARB4 target, such as a cell membrane bound isoform of a TAF 15 polypeptide), of for binding to pancreas cancer BXPC-3 cells, colon cancer HT-29 cells or stomach cancer 23132/87 cells.

Antibodies and functional fragments that bind to an antigen (e.g., BARB4 target, such as a cell membrane bound isoform of a TAF 15 polypeptide), or epitope or to a cell to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds, or that compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to an antigen or epitope or a cell, can have greater or less relative cell proliferation inhibiting or reducing activity, or greater or less relative cell apoptosis inducing or stimulating activity than BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. Antibodies and functional fragments of the invention therefore include those that bind to a cell or antigen or epitope to which BARB4 antibody, or compete with BARB4 antibody for binding to a cell or antigen or epitope, and have greater or less relative cell proliferation inhibiting or reducing activity, or greater or less relative cell apoptosis inducing or stimulating activity than BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively.

Invention antibodies therefore include those that have a sequence distinct from BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively but that retain one or more activities or functions, at least in part, of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. Exemplary activities and functions include, for example, binding to a cell to which BARB4 antibody binds; binding to an antigen (e.g., BARB4 target, such as a cell membrane bound isoform of a TAF 15 polypeptide) or epitope to which BARB4 antibody binds; competing with BARB4 antibody for binding to a cell or to an antigen (e.g., BARB4 target, such as a cell membrane bound isoform of a TAF 15 polypeptide) or epitope; inhibiting or reducing cell growth or proliferation, or stimulating or inducing cell death, lysis or apoptosis (e.g., a neoplastic, tumor or cancer, or metastasis cell); binding to one or more of an adenocarcinoma cell or a squamous cell carcinoma, such as a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, or a human adenocarcinoma, human squamous cell carcinoma, human carcinoid carcinoma, human invasive ductal carcinoma, or human germ cell carcinoma of any of stomach, lung, colon, pancreas, esophagus, prostate, breast or testis; inhibiting BXPC-3, HT-29 or 23132/87 cell growth or proliferation, or stimulating or inducing BXPC-3, HT-29 or 23132/87 cell death, lysis or apoptosis; etc.

Thus, in accordance with the invention there are also provided modified antibodies and functional fragments provided that the modified form retains, at least a part of an activity or function of unmodified or reference antibody, or functional fragment. In one embodiment, an antibody or a functional fragment thereof includes a heavy or a light chain variable region sequence with one or more amino acid additions, deletions or substitutions of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, provided said antibody or functional fragment retains at least partial activity or function of intact full length BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. In various aspects, an antibody or a functional fragment with one or more amino acid additions, deletions or substitutions of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, competes for binding to a cell or antigen (e.g., BARB4 target, such as a cell membrane bound isoform of a TAF 15 polypeptide) or epitope to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds. In other aspects, an antibody or a functional fragment with one or more amino acid deletions, substitutions or additions of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds to a cell or antigen (e.g., BARB4 target, such as a cell membrane bound isoform of a TAF 15 polypeptide) or epitope to which BARB4 antibody binds. In an additional aspect, an antibody or a functional fragment with one or more amino acid deletions, substitutions or additions of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, inhibits or reduces proliferation of a cell in which BARB4 antibody inhibits or reduces proliferation. In a further aspect, an antibody or a functional fragment with one or more amino acid deletions, substitutions or additions of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, stimulates or induces death, lysis or apoptosis of a cell in which BARB4 antibody stimulates or induces death, lysis or apoptosis. In still further particular aspects, cell growth or proliferation is inhibited, decreased or reduced at least 20%, 30%, 40%, 50%, 60%, 75%, or more relative to a control (untreated) cell, or any numerical value or range within or encompassing such percent values. In yet further particular aspects, cell death, lysis or apoptosis is at least 20%, 30%, 40%, 50%, 60%, 75%, or more relative to a control (untreated) cell, or any numerical value or range within or encompassing such percent values.

As used herein, the term “modify” and grammatical variations thereof, means that the composition deviates from a reference composition. Such modified proteins, nucleic acids and other compositions may have greater or less activity than or a distinct function from a reference unmodified protein, nucleic acid, or composition.

Modifications, which include substitutions, additions and deletions, can also be referred to as “variants.” Specific non-limiting examples of amino acid variants include antigen (e.g., BARB4 target, such as a cell membrane bound isoform of a TAF 15 polypeptide) and BARB4 antibody fragments and subsequences. Exemplary BARB4 target subsequences and fragments include, for example, a portion of the BARB4 target, such as a portion of a TAF 15 polypeptide sequence, a portion of a cell membrane bound isoform of TAF 15 polypeptide, a portion of a TAF 15 polypeptide sequence or a cell membrane bound isoform of TAF 15 polypeptide that includes a carbohydrate moiety, or a portion that binds to BARB4 antibody, as represented by antibody produced by hybridoma DSMZ Deposit No. DSM ACC2876; or heavy and light chain sequences set forth as SEQ ID NOs:1, 3, 5, 7; and 9, respectively. Exemplary BARB4 antibody subsequences and fragments include, for example, a portion of the BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, that at least partially competes with BARB4 antibody for binding to a cell or antigen, or that retains at least partial binding activity to a cell or antigen to which BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, binds, or that retains an ability to inhibit or reduce proliferation of a cell in which BARB4 antibody inhibits or reduces proliferation, or that retains an ability to stimulate or induce death, lysis or apoptosis of a cell in which BARB4 antibody stimulates or induces death, lysis or apoptosis.

As used herein, the term “fragment” or “subsequence” means a portion of the full length molecule. Thus, a fragment or subsequence of an antigen or antibody has one or more less amino acids than a full length intact reference antigen or antibody (e.g. one or more internal or terminal amino acid deletions from either amino or carboxy-termini of heavy or light chain variable or constant regions). A nucleic acid fragment has at least one less nucleotide than a full length comparison nucleic acid sequence. Fragments therefore can be any length up to the full length native molecule.

The terms “functional fragment” and “functional subsequence” when referring to an antibody refers to a portion of an antibody with a function or activity. For example, a functional fragment can retain one or more partial functions or activities as an intact reference antigen or antibody. For example, a BARB4 target that includes a portion with a sequence at least partially identical to SEQ ID NO:11 or 12, a portion of a cell membrane bound isoform of TAF 15 polypeptide, a portion of a TAF 15 polypeptide sequence or a cell membrane bound isoform of TAF 15 polypeptide that includes a carbohydrate moiety, or a portion that binds to BARB4 antibody, as represented by antibody produced by hybridoma DSMZ Deposit No. DSM ACC2876; or heavy and light chain sequences set forth as SEQ ID NOs:1, 3, 5, 7; and 9, respectively. For a BARB4 antibody, a functional subsequence can include a subsequence that competes for binding of full length intact BARB4 antibody, as represented by antibody produced by hybridoma DSMZ Deposit No. DSM ACC2876; or heavy and light chain sequences set forth as SEQ ID NOs:1, 3, 5, 7; and 9, respectively, to a cell or to an antigen or epitope, or that binds to a cell or antigen or epitope to which full length intact BARB4 antibody binds is considered a functional subsequence.

Antibody fragments, including single-chain antibodies, can include all or a portion of heavy or light chain variable region(s) (e.g., one or more CDRs, such as CDR1, CDR2 or CDR3) alone or in combination with all or a portion of one or more of the following: hinge region, CH1, CH2, and CH3 domains. Also included are antigen-binding subsequences of any combination of heavy or light chain variable region(s) (e.g., one or more CDRs, such as CDR1, CDR2 or CDR3) with a hinge region, CH1, CH2, and CH3 domains.

Exemplary antibody subsequences and fragments of the invention include Fab, Fab′, F(ab′)₂, Fv, Fd, single-chain Fv (scFv), disulfide-linked Fvs (sdFv), V_{L} and V_{H} domain fragments, trispecific (Fab₃), bispecific (Fab₂), diabody ((V_{L}-V_{L})₂ or (V_{H}-V_{L})₂), triabody (trivalent), tetrabody (tetravalent), minibody ((scFv-C_{H}3)₂), bispecific single-chain Fv (Bis-scFv), IgGdeltaCH2, scFv-Fc and (scFv)₂-Fc. Such subsequences and fragments can have binding affinity as the full length antibody, the binding specificity as the full length antibody, or one or more activities or functions of as a full length antibody, e.g., a function or activity of BARB4 antibody, as represented by antibody produced by hybridoma DSMZ Deposit No. DSM ACC2876, or heavy and light chain sequences set forth as SEQ ID NOs:1, 3, 5, 7; and 9, respectively.

Antibody subsequences and fragments can be combined. For example, a V_{L} or V_{H} subsequences can be joined by a linker sequence thereby forming a V_{L}-V_{H} chimera. In particular, a heavy chain variable sequence of BARB4 antibody as represented by antibody produced by hybridoma DSMZ Deposit No. DSM ACC2876, or heavy chain variable sequence set forth as SEQ ID NO:1, 3, 5 or 7 can be combined with a light chain variable sequence of BARB4 antibody as represented by antibody produced by hybridoma DSMZ Deposit No. DSM ACC2876, or light chain variable sequence set forth as SEQ ID NO:9. The invention therefore provides: 1) heavy chain variable sequence of BARB4 antibody, as represented by antibody produced by hybridoma DSMZ Deposit No. DSM ACC2876, or heavy chain variable sequences set forth as SEQ ID NO:1, 3, 5 or 7; and 2) light chain variable sequence of BARB4 antibody, as represented by antibody produced by hybridoma DSMZ Deposit No. DSM ACC2876, or light chain variable sequence set forth as SEQ ID NO:9 alone and in combination with each other. A combination of single-chain Fvs (scFv) subsequences can be joined by a linker sequence thereby forming a scFv-scFv chimera. Antibody subsequences and fragments include single-chain antibodies or variable region(s) alone or in combination with all or a portion of other subsequences.

Modified proteins further include amino acid substitutions. Substitutions can be conservative or non-conservative. For an antibody, substitutions may be in a constant or variable (e.g., hypervariable, such as CDR or FR) region of BARB4 antibody, as represented by antibody produced by hybridoma DSMZ Deposit No. DSM ACC2876, or heavy and light chain sequences set forth as SEQ ID NOs:1, 3, 5, 7; and 9, respectively In particular embodiments, a modified BARB4 antibody, as represented by antibody produced by hybridoma DSMZ Deposit No. DSM ACC2876, or heavy and light chain sequences set forth as SEQ ID NOs:1, 3, 5, 7; and 9, respectively, has one or a few conservative or non-conservative amino acid substitutions.

Antibody structural determinants that contribute to antigen binding, such as complemetarity determining regions (CDR, of which there are three in each heavy and light chain sequence, conveniently denoted as HC-CDR1, HC-CDR2 and HC-CDR3; and LC-CDR1, LC-CDR2 and LC-CDR3) within hypervariable regions are known to the skilled artisan. The location of additional regions, such as D- and J-regions are also known to the skilled artisan. Antibodies and subsequences thereof in which one or more CDR sequences have sufficient sequence identity to BARB4 antibody, as represented by antibody produced by hybridoma DSMZ Deposit No. DSM ACC2876, or heavy and light chain sequences set forth as SEQ ID NOs:1, 3, 5, 7; and 9, respectively, so as to retain at least partial function or activity of BARB4 antibody, as represented by antibody produced by hybridoma DSMZ Deposit No. DSM ACC2876, or heavy and light chain sequences set forth as SEQ ID NOs:1, 3, 5, 7; and 9, respectively, e.g., cell or antigen (BARB4 target that includes a portion with a sequence at least partially identical to SEQ ID NO:11 or 12, such as a cell membrane bound isoform of TAF 15 polypeptide) binding, binding affinity (e.g., K_{d}), cell proliferation inhibition, or stimulating or inducing cell apoptosis, etc.

Accordingly, amino acid substitutions in constant or variable regions of BARB4 antibody, as represented by antibody produced by hybridoma DSMZ Deposit No. DSM ACC2876, or heavy and light chain sequences set forth as SEQ ID NOs:1, 3, 5, 7; and 9, respectively, are likely to be tolerated. One or a few substitutions in a variable region outside of a CDR of BARB4 antibody, as represented by antibody produced by hybridoma DSMZ Deposit No. DSM ACC2876, or heavy and light chain sequences set forth as SEQ ID NOs:1, 3, 5, 7; and 9, respectively, is also likely to be tolerated at least to the extent that at least partial cell or antigen binding activity is retained, or partial cell proliferation inhibiting or apoptosis stimulating or inducing activity is retained. One or a few conservative substitutions in a CDR of BARB4 antibody, as represented by antibody produced by hybridoma DSMZ Deposit No. DSM ACC2876, or heavy and light chain sequences set forth as SEQ ID NOs:1, 3, 5, 7; and 9, respectively, is also likely to be tolerated at least to the extent that at least partial cell or antigen binding activity is retained (i.e., cell or antigen binding is not destroyed), or partial cell proliferation inhibiting or apoptosis stimulating or inducing activity is retained. Non-conservative substitution of many amino acids in hypervariable regions (e.g., CDRs) of BARB4 antibody, as represented by antibody produced by hybridoma DSMZ Deposit No. DSM ACC2876, or heavy and light chain sequences set forth as SEQ ID NOs:1, 3, 5, 7; and 9, respectively, is likely to affect one or more of cell or antigen binding activity, binding affinity (e.g., K_{d}), or antibody function or activity, such as cell proliferation inhibition, stimulating or inducing cell apoptosis, etc.

A “conservative substitution” is the replacement of one amino acid by a biologically, chemically or structurally similar residue. Biologically similar means that the substitution does not destroy a biological activity, e.g., cell binding or cell proliferation inhibiting or apoptosis inducing or stimulating activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

In particular embodiments, a heavy or light chain hypervariable region sequence or a region therein, such as a CDR (CDR1, CDR2 or CDR3) or FR will have 1-10, 1-5, 1-3 or fewer (e.g., 1 or 2) amino acid substitutions. In an additional embodiment, an amino acid substitution within a heavy or light chain hypervariable region sequence is not within more than one CDR. In an additional embodiment, a substitution within a heavy or light chain hypervariable region sequence is not within a CDR. In another embodiment, a substitution within a hypervariable region sequence is not within an FR.

The effect of a given modification can be readily assayed in order to identify antibodies and functional fragments retaining at least a part of the cell or antigen (BARB4 target, such as a cell membrane bound isoform of TAF 15 polypeptide) binding activity, affinity or antibody function or activity of unmodified antibody, e.g., BARB4 antibody, as represented by antibody produced by hybridoma DSMZ Deposit No. DSM ACC2876, or heavy and light chain sequences set forth as SEQ ID NOs:1, 3, 5, 7; and 9, respectively. For example, an amino acid substitution in a variable region (e.g., within or outside of CDR1, CDR2 or CDR3 of BARB4 antibody, as represented by antibody produced by hybridoma DSMZ Deposit No. DSM ACC2876, or heavy and light chain sequences set forth as SEQ ID NOs:1, 3, 5, 7 or 9) can be assayed for cell or antigen (BARB4 target, such as a cell membrane bound isoform of TAF 15 polypeptide) binding, cell proliferation inhibiting or reducing activity, inducing or stimulating cell death, lysis or apoptosis, etc.

Regional mutability analysis can be used to predict the effect of particular substitutions in complementarity determining regions (CDR) and framework regions (FR) (Shapiro et al., J Immunol. 163:259 (1999)). In brief, sequence comparison indicates a hierarchy of mutability among di- and trinucleotide sequences located within Ig intronic DNA, which predicts regions that are more or less mutable. Quantitative structure-activity relationship (QSAR) can be used to identify the nature of the antibody recognition domain and, therefore, amino acids that participate in ligand binding. Predictive models based upon OSAR can in turn be used to predict the effect of substitutions (mutations). For example, the effect of mutations on the association and dissociation rate of an antibody interacting with its antigen has been used to construct quantitative predictive models for both kinetic (Kₐ and K_{d}) constants, which in turn is used to predict the effect of other mutations on the antibody (De Genst et al., J Biol. Chem. 277:29897 (2002)). The skilled artisan can therefore use such analysis to identify amino acid substitutions of antibodies and functional fragments that are likely to result in an antibody or functional fragment that retains at least partial activity or function of non-substituted antibody or functional fragment.

Amino acid substitutions may be with the same amino acid, except that a naturally occurring L-amino acid is substituted with a D-form amino acid. Modifications therefore include one or more D-amino acids substituted for L-amino acids, or mixtures of D-amino acids substituted for L-amino acids. Modifications also include structural and functional analogues, for example, peptidomimetics having synthetic or non-natural amino acids or amino acid analogues and derivatized forms.

Modified forms further include derivatized sequences, for example, amino acids in which free amino groups form amine hydrochlorides, p-toluene sulfonyl groups, carbobenzoxy groups; the free carboxy groups from salts, methyl and ethyl esters; free hydroxl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, etc. Modifications can be produced using methods known in the art (e.g., PCR based site-directed, deletion and insertion mutagenesis, chemical modification and mutagenesis, cross-linking, etc.).

Modified forms include additions and insertions. For example, an addition can be the covalent or non-covalent attachment of any type of molecule to a protein (e.g., antibody), nucleic acid or other composition. Typically additions and insertions confer a distinct function or activity.

Additions and insertions include fusion (chimeric) polypeptide or nucleic acid sequences, which is a sequence having one or more molecules not normally present in a reference native (wild type) sequence covalently attached to the sequence. A particular example is an amino acid sequence of another protein (e.g., antibody) to produce a multifunctional protein (e.g., multispecific antibody).

In accordance with the invention, there are provided antibodies, nucleic acids, and other compositions that include a heterologous domain. Thus, a heterologous domain can consist of any of a variety of different types of small or large functional moieties. Such moieties include nucleic acid, peptide, carbohydrate, lipid or small organic compounds, such as a drug (e.g., a cell proliferative agent), metals (gold, silver), etc. A heterologous domains can be an amino acid addition or insertion.

Particular non-limiting examples of heterologous domains include, for example, tags, detectable labels and cytotoxic agents. Specific examples of tags and detectable labels include enzymes (horseradish peroxidase, urease, catalase, alkaline phosphatase, beta-galactosidase, chloramphenicol transferase); enzyme substrates; ligands (e.g., biotin); receptors (avidin); radionuclides (e.g., C¹⁴, S³⁵, P³², P³³, H³, I¹²⁵, I¹³¹, gallium-67 and 68, scantium-47, indium-111, radium-223); T7-, His-, myc-, HA- and FLAG-tags; electron-dense reagents; energy transfer molecules; paramagnetic labels; fluorophores (fluorescein, rhodamine, phycoerthrin); chromophores; chemi-luminescent (imidazole, luciferase); and bio-luminescent agents. Specific examples of cytotoxic agents include diptheria, toxin, cholera toxin and ricin.

Additional examples of heterologous domains include, for example, anti-cell proliferative agents (e.g., anti-neoplastic, anti-tumor or anti-cancer, or anti-metastasis agents). Specific non-limiting examples of anti-cell proliferative agents are disclosed herein and known in the art.

Linker sequences may be inserted between the protein (e.g., antibody), nucleic acid, or other composition and the addition or insertion (e.g., heterologous domain) so that the two entities maintain, at least in part, a distinct function or activity. Linker sequences may have one or more properties that include a flexible structure, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary (see, e.g., U.S. Pat. No. 6,087,329). Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further examples of additions include glycosylation, fatty acids, lipids, acetylation, phosphorylation, amidation, formylation, ubiquitinatation, and derivatization by protecting/blocking groups and any of numerous chemical modifications. Other permutations and possibilities will be readily apparent to those of ordinary skill in the art, and are considered to be within the scope of the invention.

The term “isolated” used as a modifier of a composition means that the composition is made by the hand of man or is separated from one or more other components in their naturally occurring in vivo environment. Generally, compositions so separated are substantially free of one or more materials with which they normally associate with in nature, for example, one or more protein, nucleic acid, lipid, carbohydrate, cell membrane. Thus, an isolated composition is substantially separated from other biological components in the cell of the organism in which the composition naturally occurs, or from the artificial medium in which it is produced (e.g., synthetically or through cell culture). For example, an isolated polypeptide is substantially separated from other polypeptides and nucleic acid and does not include a library of polypeptides or polynucleotides present among millions of polypeptide or nucleic acid sequences, such as a polypeptide, genomic or cDNA library, for example. An isolated nucleic acid is substantially separated from other polypeptides and nucleic acid and does not include a library of polypeptides or polynucleotides present among millions of polypeptide or nucleic acid sequences, such as a polypeptide, genomic or cDNA library, for example. The term “isolated” does not exclude alternative physical forms of the composition, for example, an isolated protein could include protein multimers, post-translational modifications (e.g., glycosylation, phosphorylation) or derivatized forms. The term “purified” used as a modifier of a composition refers to a composition free of most or all of the materials with which it typically associates with in nature. Thus, a protein separated from cells is considered to be substantially purified when separated from cellular components by standard methods while a chemically synthesized nucleic acid sequence is considered to be substantially purified when separated from its chemical precursors. Purified therefore does not require absolute purity. Furthermore, a “purified” composition can be combined with one or more other molecules. Thus, the term “purified” does not exclude combinations of compositions.

“Purified” proteins and nucleic acid include proteins and nucleic acids produced by standard purification methods. The term also includes proteins and nucleic acids produced by recombinant expression in a host cell as well as chemical synthesis. “Purified” can also refer to a composition in which the level of contaminants is below a level that is acceptable to a regulatory agency for administration to a human or non-human animal, for example, the Food and Drug administration (FDA).

Substantial purity can be at least about 60% or more of the molecule by mass. Purity can also be about 70% or 80% or more, and can be greater, for example, 90% or more. Purity can be less, for example, in a pharmaceutical carrier the amount of a molecule by weight % can be less than 60% but the relative proportion of the molecule compared to other components with which it is normally associated with will be greater. Purity can be determined by any appropriate method, including, for example, UV spectroscopy, chromatography (e.g., HPLC, gas phase), gel electrophoresis (e.g., silver or coomassie staining) and sequence analysis (peptide and nucleic acid).

Methods of producing polyclonal and monoclonal antibodies are known in the art. For example, BARB4 target such as a cell membrane bound isoform of TAF 15 polypeptide, or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using conventional hybridoma technology, splenocytes from immunized animals that respond to BARB4 target can be isolated and fused with myeloma cells. Monoclonal antibodies produced by the hybridomas can be screened for reactivity with BARB4 target.

Antibodies that compete with BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, for binding to a cell or antigen can be screened and identified using a conventional competition binding assays. Screened antibodies are selected based upon an ability to compete with BARB4 antibody, as represented by DSMZ Deposit No. DSM ACC287, or heavy and light chain variable region sequences set forth as SEQ ID NOs:1, 3, 5, 7; and 9, respectively, for binding to a cell or antigen. The ability of an antibody to compete with BARB4 antibody for binding to a cell or antigen, or to inhibit, prevent or block binding of BARB4 antibody to a cell or antigen, can be determined by various assays know in the art, including enzyme linked immunosorbent assay (ELISA).

Proteins and antibodies, subsequences and fragments thereof, as well as other modified sequences can be produced by genetic methodology. Such techniques include expression of all or a part of the gene encoding the protein or antibody into a host cell such as Cos cells or E. coli. Such host cells can express full length or a fragment, for example, an scFv (see, e.g., Whitlow et al., In: Methods: A Companion to Methods in Enzymology 2:97 (1991), Bird et al., Science 242:423 (1988); and U.S. Pat. No. 4,946,778). Antibodies and functional fragments, and nucleic acid sequences can also be produced by chemical synthesis using methods known to the skilled artisan, for example, an automated peptide synthesis apparatus (see, e.g., Applied Biosystems, Foster City, Calif.). Antibodies and functional fragments can be screened or selected using various assays know in the art, such as enzyme linked immunosorbent assay (ELISA), phage display, protein-mRNA link via ribosome and mRNA display, display on yeast, bacteria, mammalian cells or retroviruses, microbead via in vitro compartmentalization, protein-DNA display, growth selection via yeast 2-hybrid, protein fragment complementation (Hoogenboom, R., Nature Biotechnol. 23:1105 (2005)).

Cells or antigen (BARB4 target such as a cell membrane bound isoform of TAF 15 polypeptide) suitable for generating antibodies can be produced by any of a variety of standard protein purification or recombinant expression techniques known in the art. For example, BARB4 target is present on cells, such as BXPC-3 cells (ATCC Deposit No. CRL-1687; P.O. Box 1549 Manassas, Va., 20108, USA) or 23132/87 cells (DSMZ Deposit No. ACC 201; Deutsche Sammlung von Mikroorganismen und Zellkulturen GmbH (German Collection of Microorganisms and Cell Cultures), Inhoffenstrase 7 B 38124 Braunschweig, Germany). Accordingly, whole cells, or preparations, cell extracts or fractions of such cells can be used to immunize animals in order to produce antibodies that compete with BARB4 antibody, as represented by antibody produced by hybridoma DSMZ Deposit No. DSM ACC2876, or heavy and light chain sequences set forth as SEQ ID NOs:1, 3, 5, 7; and 9, respectively, for binding of to a cell or antigen, or that bind to a cell or antigen (e.g., BARB4 target, such as a cell membrane bound isoform of TAF 15 polypeptide) to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds, for example.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of BARB4 antigen preparation, and may be at regular or irregular intervals.

Animals include those genetically modified to include human IgG gene loci, which can therefore be used to produce human antibodies. Transgenic animals with one or more human immunoglobulin genes that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598). An overview of the technology for producing human antibodies is described in Lonberg and Huszar (Int. Rev. Immunol. 13:65 (1995)).

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKeam, and Bechtol (eds.), 1980, and Harlow et al., Antibodies: A Laboratory Manual, Cold Spring Harbor Laboratory Press, 2nd ed. 1988).

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enzymol. 1:422 (1967)). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988). Other methods of cleaving antibodies, such as separation of heavy chains to form monovalent light-heavy chain fragments, further cleavage of fragments, or other enzymatic or chemical may also be used.

Modified antibodies and functional fragments having altered characteristics, such as increased binding affinity, can be produced using methods known to the skilled artisan art. For example, affinity maturation techniques can be used to improve antibody binding affinity (US 2004/0162413 A1; U.S. Pat. Nos. 6,656,467, 6,531,580, 6,590,079 and 5,955,358; Fiedler et al., Protein Eng. 15:931 (2002); Pancook et al., Hybrid. Hybridomics 20:383 (2001); Daugherty et al., Protein Eng. 11:825 (1998); Wu et al., Proc. Nat'l Acad. Sci. USA 95:6037 (1998); and Osbourn et al., Immunotechnology 2:181 (1996)).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al, Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Suitable techniques that additionally may be employed in antibody methods include affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

In accordance with the invention, further provided are methods of producing antibodies and functional fragments. In one embodiment, a method includes administering an antigen (e.g., a BARB4 target such as a cell membrane bound isoform of TAF 15 polypeptide, a TAF polypeptide that binds to a BARB4 antibody or a TAF polypeptide that includes a carbohydrate moiety), or cell expressing an antigen (BARB4 target such as a TAF 15 polypeptide expressed by a tumor or cancer cell), to an animal, screening the animal for expression of an antibody that binds to the an antigen (e.g., a BARB4 target) or cell expressing an antigen (e.g., a BARB4 target), selecting an animal that produces an antibody that binds to antigen (e.g., a BARB4 target) or cell expressing the antigen (e.g., a BARB4 target), and isolating the antibody from the selected animal. In another embodiment, a method includes administering an antigen (e.g., a BARB4 target such as a cell membrane bound isoform of TAF 15 polypeptide, a TAF polypeptide that binds to a BARB4 antibody or a TAF polypeptide that includes a carbohydrate moiety), or cell expressing an antigen (BARB4 target such as a TAF 15 polypeptide expressed by a tumor or cancer cell), to an animal capable of expressing a human immunoglobulin; isolating spleen cells from an animal that produces antibody that binds to the antigen (e.g., a BARB4 target) or cell expressing antigen (e.g., a BARB4 target), fusing the spleen cells with a myeloma cell to produce a hybridoma, and screening the hybridoma for expression of an antibody that binds to antigen (e.g., a BARB4 target) or cell expressing antigen (e.g., a BARB4 target).

In accordance with the invention, there are provided host cells that express antigen, antibodies, and functional fragments of the antibodies as set forth herein. In particular embodiments, host cells are purified or isolated, and optionally have not been transformed with a nucleic acid that encodes the expressed antigen, antibody or functional fragment. In additional embodiments, a host cell expresses an antigen that has a sequence with 50%, 60%, 70%, 75%, 80%, 85%, 90%, 95%, 96%, 97%, 98%, or more sequence identity to a TAF polypeptide, such as SEQ ID NO:11 or 12. In further embodiments, a host cell expresses a BARB4 antibody, antibody or functional fragment that includes a heavy or light chain sequence with 50%, 60%, 70%, 75%, 80%, 85%, 90%, 95%, 96%, 97%, 98%, or more sequence identity to BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively. In still further embodiments, a host cell expresses a heavy or light chain sequence with at least 80-85%, 85-90%, 90-95%, 95-100% identity to one or more CDRs in heavy chain variable region sequence or light chain variable region sequence BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively.

In accordance with the invention, there are provided isolated and purified nucleic acids. Nucleic acids of the invention include, among other things, nucleic acid sequences 1) encoding antigen (e.g., a BARB4 target such as a cell membrane bound isoform of TAF 15 polypeptide), 2) encoding antibodies and functional fragments that are structurally or functionally related to BARB4 antibody, as represented by antibody produced by hybridoma DSMZ Deposit No. DSM ACC2876; or heavy and light chain sequences set forth as SEQ ID NOs:1, 3, 5, 7; and 9, respectively; 3) encode SEQ ID NOs:1, 3, 5, 7 or 9, or antibodies and functional fragments that include all or a portion of a sequence of SEQ ID NOs:1, 3, 5, 7 or 9 (e.g., one or more CDRs); 4) that exhibit a degree of complementarity or identity with nucleic acid sequences encoding antigen (e.g., a BARB4 target such as a cell membrane bound isoform of TAF 15 polypeptide); 5) that exhibit a degree of complementarity or identity with nucleic acid sequences encoding antibodies and functional fragments with sequence identity to BARB4 antibody, as represented by antibody produced by hybridoma DSMZ Deposit No. DSM ACC2876, or heavy and light chain sequences set forth as SEQ ID NOs:1, 3, 5, 7; and 9, respectively; 6) that hybridize to sequences encoding antigen (e.g., a BARB4 target such as a cell membrane bound isoform of TAF 15 polypeptide); and 7) that hybridize to sequences encoding antibodies and functional fragments that have sequence identity to BARB4 antibody, as represented by antibody produced by hybridoma DSMZ Deposit No. DSM ACC2876, or heavy and light chain sequences set forth as SEQ ID NOs:1, 3, 5, 7; and 9, respectively.

In particular embodiments, a nucleic acid sequence encodes a heavy or light chain sequence of BARB4 antibody, as represented by antibody produced by hybridoma DSMZ Deposit No. DSM ACC2876, or heavy and light chain sequences set forth as S SEQ ID NOs:1, 3, 5, 7; and 9, respectively, or a functional fragment thereof. In another embodiment, a nucleic acid sequence is 75-100% complementary or identical to a nucleic acid sequence that encodes SEQ ID NO:1, 3, 5 or 7. In a further embodiment, a nucleic acid sequence is 75-100% complementary or identical to a nucleic acid sequence that encodes SEQ ID NO:9.

Nucleic acid sequences of BARB4, heavy and light chain variable region sequences, as represented by SEQ ID NOs:2, 4, 6 and 8; and 10, respectively, are shown.

Nucleic acid sequence of BARB4 heavy chain (VH;
SEQ ID NO:2):
CAGGTGCAGC TGGTGGAGTC TGGGGGAGGC GTGGTCCAGC
CTGGGAGGTC CCTAAGACTC TCCTGTGCAG CCTCTGGATT
CAGGTTCACT ACACACGGCA TGCACTGGGT CCGCCAGGCT
CCAGGCAAGG GGCTGGAGTG GGTGGCAGTT ATATCATATA
ATGGAAGAAA CAAATACTAT GCAGACTACG TGAACGGCCG
ATTCACCATC TCCAGAGACG ATTCCAGGGA CACGGTGTTT
CTGCAAATGA ACAGCCTGAG ACCTGAGGAC ACGGCTATGT
ACTACTGTGC GAAAGTTAGG GGCGATGGCT ACGGTGACTA
TGGCTACTTT GACTACTGGG GCCACGGAAC CCTGGTCAGC
GTCTCCTCA
Nucleic acid sequence of BARB4 heavy chain (4.1
VH; SEQ ID NO:4):
GAGGTGCAGC TGGTGGAGTC TGGGGGAGGC GTGGTCCAGC
CTGGGAGGTC CCTAAGACTC TCCTGTGCAG CCTCTGGATT
CAGGTTCACT ACACACGGCA TGCACTGGGT CCGCCAGGCT
CCAGGCAAGG GGCTGGAGTG GGTGGCAGTT ATATCATATA
ATGGAAGAAA CAAATACTAT GCAGACTACG TGAACGGCCG
ATTCACCATC TCCAGAGACG ATTCCAGGGA CACGGTGTTT
CTGCAAATGA ACAGCCTGAG ACCTGAGGAC ACGGCTATGT
ACTACTGTGC GAAAGTTAGG GGCGATGGCT ACGGTGACTA
TGGCTACTTT GACTACTGGG GCCACGGAAC CCTGGTCAGC
GTCTCCTCA
Nucleic acid sequence of BARB4 heavy chain (4.2
VH; SEQ ID NO:6):
GAAGTGCAGC TGGTGGAAAG CGGCGGCGGC CTGGTGCAGC
CGGCCGGGTC CCTGAGACTC TCCTGTGCAG CCTCTGGATT
CAGGTTCACT ACACACGGCA TGCACTGGGT CCGCCAGGCT
CCAGGCAAGG GGCTGGAGTG GGTGGCAGTT ATATCATATA
ATGGAAGAAA CAAATACTAT GCAGACTACG TGAACGGCCG
ATTCACCATC TCCAGAGACG ATTCCAGGGA CACGGTGTTT
CTGCAAATGA ACAGCCTGAG ACCTGAGGAC ACGGCTATGT
ACTACTGTGC GAAAGTTAGG GGCGATGGCT ACGGTGACTA
TGGCTACTTT GACTACTGGG GCCACGGAAC CCTGGTCAGC
GTCTCCTCA
Nucleic acid sequence of BARB4 heavy chain (4.3
VH; SEQ ID NO:8):
CAGGTGCAGC TGGTGGAGTC TGGGGGAGGC GTGGTCCAGC
CTGGGAGGTC CCTAAGACTC TCCTGTGCAG CCTCTGGATT
CAGGTTCACT ACACACGGCA TGCACTGGGT CCGCCAGGCT
CCAGGCAAGG GGCTGGAGTG GGTGGCAGTT ATATCATATA
ATGGAAGAAA CAAATACTAT GCAGACTACG TGAACGGCCG
ATTCACCATC TCCAGAGACG ATTCCAGGGA CACGGTGTTT
CTGCAAATGA ACAGCCTGAG ACCTGAGGAC ACGGCTATGT
ACTACTGTGC GAAAGTTAGG GGCGATGGCT ACGGTGACTA
TGGCTACTTT GACTACTGGG GCCACGGAAC CCTGGTCACC
GTCTCCTCA
Nucleic acid sequence of BARB4 light chain (VL;
SEQ ID NO:10):
CAGTCTGCCC TGACTCAGCC TCGCTCAGTG TCCGGGTCTC
CTGGACAGTC AGTCACCATC TCCTGCACTG GAACTTATAA
CTATGTCTCC TGGTACCAAC AGCACCCAGG CAAAGCCCCC
AAACTCATGA TTTTTGATGT CAGTAGGCGG TCCTCAGGGG
TCCCTGATCG CTTCTCTGGC TCCAAGTCTG GCAACACGCC
CTCCCTGACC ATCTCTGGGC TCCAGGCTGA GGATGAGGCT
GATTATTACT GCTGCTCATA TGGAGGCACC TACCTTTATG
TCTTCGGAAC TGGGACTACG GTCACCGTCC TTGGTCAG

Proteins, such as antigens and antibodies that include amino acid substitutions, additions or deletions can be encoded by a nucleic acid. Consequently, nucleic acid sequences encoding proteins that include amino acid substitutions, additions or deletions are also provided.

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular embodiments, a nucleic acid sequence has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. Shorter polynucleotides are commonly referred to as “oligonucleotides” or “probes” of single- or double-stranded DNA. However, there is no upper limit to the length of such oligonucleotides.

Polynucleotides include L- or D-forms and mixtures thereof, which additionally may be modified to be resistant to degradation when administered to a subject. Particular examples include 5′ and 3′ linkages resistant to endonucleases and exonucleases present in various tissues or fluids of a subject.

In accordance with the invention there are provided nucleic acid sequences that hybridize to a nucleic acid that encodes all or a fragment of an antigen (e.g., a BARB4 target such as a cell membrane bound isoform of TAF 15 polypeptide), or BARB4 antibody, as represented by antibody produced by hybridoma DSMZ Deposit No. DSM ACC2876, or heavy and light chain sequences set forth as SEQ ID NOs:1, 3, 5, 7; and 9, respectively. In one embodiment, a nucleic acid sequence specifically hybridizes to a nucleic acid encoding an antigen (e.g., a BARB4 target such as a cell membrane bound isoform of TAF 15 polypeptide) or a subsequence thereof. In another embodiment, a nucleic acid sequence specifically hybridizes to a nucleic acid encoding SEQ ID NO:1, 3, 5 or 7 or a portion thereof. In a further embodiment, a nucleic acid sequence specifically hybridizes to a nucleic acid encoding SEQ ID NO:9 or a portion thereof. In additional embodiments, a nucleic acid sequence is at least 75-100% complementary or homologous to a nucleic acid sequence that encodes all or a subsequence or fragment of an antigen (e.g., a BARB4 target such as a cell membrane bound isoform of TAF 15 polypeptide), or a BARB4 antibody, as represented by antibody produced by hybridoma DSMZ Deposit No. DSM ACC2876, or heavy and light chain sequences set forth as SEQ ID NOs:1, 3, 5, 7; and 9, respectively.

The term “hybridize” and grammatical variations thereof refer to the binding between nucleic acid sequences. Hybridizing sequences will generally have more than about 50% homology (e.g., 50%, 60%, 70%, 80%, 90%, or more identity) to a reference nucleic acid or a sequence complementary to a reference sequence. Hybridizing sequences that are 100% or fully complementary to a reference sequence, for example, to a nucleic acid that encodes an amino acid sequence of a reference sequence, exhibit 100% base pairing with no mismatches. The hybridization region between hybridizing sequences typically is at least about 12-15 nucleotides, 15-20 nucleotides, 20-30 nucleotides, 30-50 nucleotides, 50-100 nucleotides, 100 to 200 nucleotides or more, or any numerical value or range within or encompassing such lengths.

In accordance with the invention, there are further provided antisense polynucleotides, small interfering RNA, and ribozyme nucleic acid. In particular embodiments, an antisense polynucleotide, small interfering RNA, or ribozyme nucleic acid specifically hybridizes to a nucleic acid sequence encoding an antigen (e.g., a BARB4 target such as a cell membrane bound isoform of TAF 15 polypeptide) or a subsequence thereof and optionally reduces expression of the antigen, or specifically hybridizes to SEQ ID NOs:1, 3, 5, 7 or 9, or a portion thereof, and optionally reduces expression of SEQ ID NOs:1, 3, 5, 7 or 9. In another embodiment, an antisense polynucleotide, small interfering RNA, or ribozyme nucleic acid is at least 60% or more (e.g., 65%, 70%, 75%, 80%, 85%, 90%, 95%, etc.) complementary or homologous to a nucleic acid sequence that encodes antigen (e.g., a BARB4 target such as a cell membrane bound isoform of TAF 15 polypeptide), SEQ ID NOs:1, 3, 5, 7 or 9, or a subsequence thereof. Antisense polynucleotides can have a length from about 10-20, 20-30, 30-50, 50-100, 100-150, 150-200, 200-250, 250-300, 300-400, 400-500, 500-1000, 1000-2000 nucleotides, or any numerical value or range within or encompassing such lengths.

As used herein, the term “antisense” refers to a polynucleotide or peptide nucleic acid capable of binding to a specific DNA or RNA sequence. Antisense includes single, double, triple or greater stranded RNA and DNA polynucleotides and peptide nucleic acids (PNAs) that bind RNA transcript or DNA. Particular examples include RNA and DNA antisense that binds to sense RNA. For example, a single stranded nucleic acid can target a protein transcript that participates in metabolism, catabolism, removal or degradation of glycogen from a cell (e.g., mRNA). Antisense molecules are typically 95-100% complementary to the sense strand but can be “partially” complementary, in which only some of the nucleotides bind to the sense molecule (less than 100% complementary, e.g., 95%, 90%, 80%, 70% and sometimes less), or any numerical value or range within or encompassing such percent values.

Triplex forming antisense can bind to double strand DNA thereby inhibiting transcription of the gene. Oligonucleotides derived from the transcription initiation site of the gene, e.g., between positions −10 and +10 from the start site, are one particular example.

Short interfering RNA (referred to as siRNA or RNAi) for inhibiting gene expression is known in the art (see, e.g., Kennerdell et al., Cell 95:1017 (1998); Fire et al., Nature, 391:806 (1998); WO 02/44321; WO 01/68836; WO 00/44895, WO 99/32619, WO 01/75164, WO 01/92513, WO 01/29058, WO 01/89304, WO 02/16620; and WO 02/29858). RNAi silencing can be induced by a nucleic acid encoding an RNA that forms a “hairpin” structure or by expressing RNA from each end of an encoding nucleic acid, making two RNA molecules that hybridize.

Ribozymes, which are enzymatic RNA molecules that catalyze the specific cleavage of RNA can be used to inhibit expression of the encoded protein. Ribozymes form sequence-specific hybrids with complementary target RNA, which is then cleaved. Specific examples include engineered hammerhead motif ribozyme molecules that can specifically and efficiently catalyze endonucleolytic cleavage of sequences encoding a protein that participates in metabolism, catabolism, removal or degradation of glycogen, for example.

Antisense, ribozymes, RNAi and triplex forming nucleic acid are referred to collectively herein as “inhibitory nucleic acid” or “inhibitory polynucleotides.” Such inhibitory nucleic acid or polynucleotides can inhibit or reduce expression of the sequence to which it binds or targets, and consequently, encoded protein as appropriate.

Inhibitory polynucleotides do not require expression control elements in order to function in vivo. Inhibitory polynucleotides can be absorbed by the cell or enter the cell via passive diffusion. Inhibitory polynucleotides can optionally be introduced into a cell using a vector. Inhibitory polynucleotides may be encoded by a nucleic acid so that it is transcribed. Furthermore, a nucleic acid encoding an inhibitory polynucleotide may be operatively linked to an expression control element for sustained or increased expression of the encoded antisense in cells or in vivo. Inhibitory nucleic acid can be designed based upon protein and nucleic acid sequences disclosed herein or available in the database.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode, modified forms and variants thereof. Other examples are nucleic acids complementary to a sequence that encodes

Nucleic acid deletions (subsequences and fragments) can have from about 10 to 25, 25 to 50 or 50 to 100 nucleotides. Such nucleic acids are useful for expressing polypeptide subsequences, for genetic manipulation (as primers and templates for PCR amplification), and as probes to detect the presence or an amount of a sequence encoding a protein (e.g., via hybridization), in a cell, culture medium, biological sample (e.g., tissue, organ, blood or serum), or in a subject.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

In accordance with the invention, there are further provided vectors that comprise nucleic acid sequences of the invention. In one embodiment, a vector includes a nucleic acid sequence encoding an antibody or functional fragment as set forth herein. In another embodiment, a vector includes a nucleic acid sequence encoding

Vectors include viral, prokaryotic (bacterial) and eukaryotic (plant, fungal, mammalian) vectors. Vectors can be used for expression of nucleic acids in vitro or in vivo. Such vectors, referred to as “expression vectors,” are useful for introducing nucleic acids, including nucleic acids that encode antigen (e.g., a BARB4 target such as a cell membrane bound isoform of TAF 15 polypeptide), and BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, subsequences and fragments thereof, nucleic acids that encode modified forms or variants of BARB4 antibody, as represented by antibody produced by hybridoma DSMZ Deposit No. DSM ACC2876, or heavy and light chain variable region sequences set forth as SEQ ID NOs:1, 3, 5, 7; and 9, respectively, nucleic acids that encode inhibitory nucleic acid, and expressing the encoded protein or inhibitory nucleic acid (e.g., in solution or in solid phase), in cells or in a subject in vivo.

Vectors can also be used for manipulation of nucleic acids. For genetic manipulation “cloning vectors” can be employed, and to transcribe or translate the inserted nucleic acid.

A vector generally contains an origin of replication for propagation in a cell in vitro or in vivo. Control elements, including expression control elements, present within a vector, can be included to facilitate transcription and translation, as appropriate.

Vectors can include a selection marker. A “selection marker” is a gene that allows for the selection of cells containing the gene. “Positive selection” refers to a process in which cells that contain the selection marker survive upon exposure to the positive selection. Drug resistance is one example of a positive selection marker-cells containing the marker will survive in culture medium containing the selection drug, and cells lacking the marker will die. Selection markers include drug resistance genes such as neo, which confers resistance to G418; hygr, which confers resistance to hygromycin; and puro, which confers resistance to puromycin. Other positive selection marker genes include genes that allow identification or screening of cells containing the marker. These genes include genes for fluorescent proteins (GFP and GFP-like chromophores, luciferase), the lacZ gene, the alkaline phosphatase gene, and surface markers such as CD8, among others. “Negative selection” refers to a process in which cells containing a negative selection marker are killed upon exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine.

Viral vectors include those based upon retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Additional viral vectors useful for expression include parvovirus, Norwalk virus, coronaviruses, paramyxo- and rhabdoviruses, togavirus (e.g., sindbis virus and semliki forest virus) and vesicular stomatitis virus (VSV).

A nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

Tissue-specific expression control elements include promoters and enhancers active in hyperproliferative cells, such as cell proliferative disorders including neoplasias, tumors and cancers, and metastasis. Particular non-limiting examples of such promoters are hexokinase II, COX-2, alpha-fetoprotein, carcinoembryonic antigen, DE3/MUC1, prostate specific antigen, C-erB2/neu, telomerase reverse transcriptase and hypoxia-responsive promoter.

For bacterial expression, constitutive promoters include T7, as well as inducible promoters such as pL of bacteriophage λ, plac, ptrp, ptac (ptrp-lac hybrid promoter). In insect cell systems, constitutive or inducible promoters (e.g., ecdysone) may be used. In yeast, constitutive promoters include, for example, ADH or LEU2 and inducible promoters such as GAL (see, e.g., Ausubel et al., In: Current Protocols in Molecular Biology, Vol. 2, Ch. 13, ed., Greene Publish. Assoc. & Wiley Interscience, 1988; Grant et al., In: Methods in Enzymology, 153:516-544 (1987), eds. Wu & Grossman, 1987, Acad. Press, N.Y.; Glover, DNA Cloning, Vol. II, Ch. 3, IRL Press, Wash., D.C., 1986; Bitter, In: Methods in Enzymology, 152:673-684 (1987), eds. Berger & Kimmel, Acad. Press, N.Y.; and, Strathern et al, The Molecular Biology of the Yeast Saccharomyces eds. Cold Spring Harbor Press, Vols. I and II (1982)).

For mammalian expression, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

In accordance with the invention, there are provided host cells transformed or transfected with nucleic acids and vectors of the invention. In one embodiment, a cell is stably or transiently transformed with a nucleic acid that encodes an antigen (e.g., a BARB4 target such as a cell membrane bound isoform of TAF 15 polypeptide), or a subsequence thereof. In another embodiment, a cell is stably or transiently transformed with a nucleic acid that encodes an antibody, a functional fragment, a heavy or light chain sequence, or a portion of a heavy or light chain sequence (e.g., a variable region, or one or more CDRs). In another embodiment, a host cell is stably or transiently transformed with an antisense or inhibitory nucleic acid.

Host cells include but are not limited to prokaryotic and eukaryotic cells such as bacteria, fungi (yeast), plant, insect, and animal (e.g., mammalian, including primate and human) cells. The cells may be a primary cell isolate, cell culture (e.g., passaged, established or immortalized cell line), or part of a plurality of cells, or a tissue or organ ex vivo or in a subject (in vivo). For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression.

The term “transformed” or “transfected” when use in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid can be stably or transiently transfected or transformed (expressed) in the cell and progeny thereof. Host cells therefore include those that stably or transiently express antibody, functional fragment or nucleic acid. The cell(s) can be propagated and the introduced antibody expressed, or nucleic acid transcribed. A progeny of a transfected or transformed cell may not be identical to the parent cell, since there may be mutations that occur during replication.

Typically, cell transfection or transformation employs a “vector,” which refers to a plasmid, virus, such as a viral vector, or other vehicle known in the art that can be manipulated by insertion or incorporation of a nucleic acid.

A viral particle or vesicle can be designed to be targeted to particular cell types (e.g., hyperproliferating cells) by inclusion of a protein on the surface that binds to a target cell ligand or receptor. Alternatively, a cell type-specific promoter and/or enhancer can be included in the vector in order to express the nucleic acid in target cells. Thus, the viral particle or vesicle itself, viral vector, or a protein on the viral surface can be made to target cells for transfection or transformation in vitro, ex vivo or in vivo.

Introduction of compositions (e.g., protein and nucleic acid) into target cells (e.g., host cells) can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrylate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means of delivery into cells, tissue or organs, in vitro, in vivo and ex vivo are included.

The invention includes in vivo methods. For example, a cell such as an undesirably proliferating cell or cell proliferative disorder to which BARB4 antibody or functional fragment binds can be present in a subject, such as a mammal (e.g., a human subject). A subject having such cells may therefore be treated by administering, for example, an antibody, or subsequence or fragment thereof, that binds to such cells.

In accordance with the invention, there are provided methods of treating undesirable cell proliferation or a cell proliferative or cellular hyperproliferative disorder in a subject. Such methods can be practiced with any of the antibodies, functional fragments, modified and variant forms set forth herein. In one embodiment, a method includes administering to a subject an amount of BARB4 antibody, as represented by antibody produced by hybridoma DSMZ Deposit No. DSM ACC2876, or heavy and light chain sequences set forth as SEQ ID NOs:1, 3, 5, 7; and 9, respectively, effective to treat the undesirable cell proliferation or a cell proliferative or cell hyperproliferative disorder in the subject. In another embodiment, a method includes administering to a subject an amount of antigen (e.g., a BARB4 target such as a cell membrane bound isoform of TAF 15 polypeptide), effective to treat the undesirable cell proliferation or a cell proliferative or cell hyperproliferative disorder in the subject.

As used herein, the terms “cell proliferative disorder” and “cellular hyperproliferative disorder” and grammatical variations thereof, when used in reference to a cell, tissue or organ, refers to any undesirable, excessive or abnormal cell, tissue or organ growth, proliferation, differentiation or survival. A hyperproliferative cell denotes a cell whose growth, proliferation, or survival is greater than desired, such as a reference normal cell, e.g., a cell that is of the same tissue or organ but is not a hyperproliferative cell, or a cell that fails to differentiate normally. Undesirable cell proliferation and hyperproliferative disorders include diseases and physiological conditions, both benign hyperplastic conditions characterized by undesirable, excessive or abnormal cell numbers, cell growth, cell proliferation, cell survival or differentiation in a subject. Specific examples of such disorders include metastatic and non-metastatic neoplasia, tumors and cancers (malignancies).

In various embodiments, a method includes administering to a subject a BARB4 antibody, as represented by antibody produced by hybridoma DSMZ Deposit No. DSM ACC2876, or heavy and light chain sequences set forth as SEQ ID NOs:1, 3, 5, 7; and 9, respectively, or an antigen (e.g., a BARB4 target such as a cell membrane bound isoform of TAF 15 polypeptide), in an amount effective to treat the cell proliferative or cellular hyperproliferative disorder in the subject. In particular aspects, the disorder is a neoplasia, tumor or metastatic or non-metastatic cancer (malignancy). In additional aspects, the disorder affects or is present in part at least in breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, or the hematopoetic system.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumor, cancer or neoplasia to other sites, locations or regions within the subject, in which the sites, locations or regions are distinct from the primary tumor or cancer.

Invention methods can be used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression. Thus, methods of the invention include, among other things, 1) reducing or inhibiting growth, proliferation, mobility or invasiveness of tumor or cancer cells that potentially or do develop metastases (e.g, disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

Neoplasias, tumors and cancers include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or haematopoietic neoplastic disorders (e.g., myeloma, lymphoma or leukemia). In particular aspects, a neoplasia, tumor or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, or uterine adenocarcinoma.

Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission.

Neoplasias, tumors and cancers can arise from a multitude of primary tumor types, including but not limited to breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, and the hematopoetic system, and may metastasize to secondary sites.

A “solid neoplasia, tumor or cancer” refers to neoplasia, tumor or cancer (e.g., metastasis) that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure. Melanoma refers to malignant tumors of melanocytes and other cells derived from pigment cell origin that may arise in the skin, the eye (including retina), or other regions of the body. Additional carcinomas can form from the uterine/cervix, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma

Specific non-limiting examples of neoplasias, tumors and cancers amenable to treatment include malignant and non-malignant neoplasias, tumors and cancers, and metastasis. In particular, gastric (stomach) tissue, lung squamous cell carcinoma, and lung adenocarcinoma cell of any stage (e.g., stages IA, IB, IIA, IIB, IIIA, IIIB or IV) or grade (e.g., grades G1, G2 or G3). Additional non-limiting examples include adenocarcinoma or a squamous cell carcinoma, such as a stomach adenocarcinoma, a lung adenocarcinoma, a pancreas adenocarcinoma, a colon adenocarcinoma, a breast adenocarcinoma, or an esophagus squamous cell carcinoma. Further non-limiting examples include a human adenocarcinoma, human squamous cell carcinoma, human carcinoid carcinoma, human invasive ductal carcinoma, human germ cell carcinoma in any of stomach, lung, colon, pancreas, esophagus, prostate, breast or testis.

A “liquid neoplasia, tumor or cancer” refers to a neoplasia, tumor or cancer of the reticuloendothelial or hematopoetic system, such as a lymphoma, myeloma, or leukemia, or a neoplasia that is diffuse in nature. Particular examples of leukemias include acute and chronic lymphoblastic, myeloblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment protocol, regimen, process or remedy, treating does not require that the desired physiologic response or outcome be achieved in each and every patient or patient population. Accordingly, a given patient or patient population may fail to respond or respond inadequately to treatment.

Methods of the invention may be practiced by any mode of administration or by any route, systemic, regional and local administration. Exemplary administration routes include intravenous, intrarterial, intradermal, intramuscular, subcutaneous, intra-pleural, transdermal (topical), transmucosal, intra-cranial, intra-spinal, intra-ocular, rectal, oral (alimentary) and mucosal.

Methods of the invention include, among other things, methods that provide a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i.e., a therapeutic benefit or a beneficial effect.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, stimulating, inducing or increasing neoplasia, tumor or cancer cell lysis or apoptosis or inhibiting neoplasia, tumor or cancer proliferation, growth or metastasis. An invention method may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur after cell lysis or apoptosis of the neoplasia, tumor or cancer, or metastasis.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subjects well being, such as increased energy, appetite, psychological well being, are all particular non-limiting examples of therapeutic benefit. A therapeutic benefit or improvement therefore can also include a subjective improvement in the quality of life of a treated subject.

In various embodiments, a method reduces or decreases neoplasia, tumor or cancer, or metastasis volume, inhibits or prevents an increase in neoplasia, tumor or cancer volume, inhibits or delays neoplasia, tumor or cancer progression or worsening, stimulates neoplasia, tumor or cancer, or metastasis cell lysis or apoptosis, or inhibits, reduces, decreases or delays neoplasia, tumor or cancer proliferation or metastasis. In an additional embodiment, a method prolongs or extends lifespan of the subject. In a further embodiment, a method improves the quality of life of the subject.

Examination of a biopsied sample containing a neoplasia, tumor or cancer, or metastasis (e.g., blood or tissue sample), can establish neoplastic, tumor or cancer cell volume or cell numbers, and therefore whether a reduction or stabilization in mass or numbers of neoplastic, tumor or cancer cells or inhibition of neoplasia, tumor or cancer cell proliferation, growth or survival (apoptosis) has occurred. For a solid neoplasia, tumor or cancer, invasive and non-invasive imaging methods can ascertain neoplasia, tumor or cancer size or volume. Examination of blood or serum, for example, for populations, numbers and types of cells (e.g., hematopoetic cellular hyperproliferative disorders) can establish whether a reduction or stabilization in mass or numbers of neoplastic, tumor or cancer cells or inhibition of neoplastic, tumor or cancer proliferation, growth or survival (apoptosis) has occurred.

Invention compositions and methods can be combined with any other treatment or therapy that provides a desired effect. In particular, treatments and therapies that have been characterized as having an anti-cell proliferative activity or function are applicable. Exemplary treatments and therapies include anti-cell proliferative or immune enhancing agents or drugs.

The treatments and therapies can be performed prior to, substantially contemporaneously with any other methods of the invention, for example, an anti-cell proliferative or anti-cellular hyperproliferative disorder (e.g., a neoplasia, tumor or cancer, or metastasis).

The invention therefore provides combination methods in which the methods of the invention, in which any of the antibodies, functional fragments, and modified and variant forms, are used in a combination with any therapeutic regimen, treatment protocol or composition, such as an anti-cell proliferative rotocol, agent or drug set forth herein or known in the art. In one embodiment, a method includes administering BARB4 antibody, as represented by antibody produced by hybridoma DSMZ Deposit No. DSM ACC2876, or heavy and light chain sequences set forth as SEQ ID NOs:1, 3, 5, 7; and 9, respectively, and an anti-cell proliferative or immune enhancing treatment, agent or drug. In another embodiment, a method includes administering an antigen (e.g., a BARB4 target such as a cell membrane bound isoform of TAF 15 polypeptide), and an anti-cell proliferative or immune enhancing treatment, agent or drug. The anti-cell proliferative or immune enhancing treatment, agent or drug can be administered prior to, substantially contemporaneously with or following administration of BARB4 antibody, as represented by antibody produced by hybridoma DSMZ Deposit No. DSM ACC2876, or heavy and light chain sequences set forth as SEQ ID NOs:1, 3, 5, 7; and 9, respectively, or administration of antigen (e.g., a BARB4 target such as a cell membrane bound isoform of TAF 15 polypeptide).

As used herein, an “anti-cell proliferative,” “anti-neoplastic,” “anti-tumor,” or “anti-cancer” treatment, therapy, activity or effect means any therapy, treatment regimen, agent, drug, protocol or process that is useful in treating pathologies, adverse symptoms or complications associated with or caused by abnormal or undesirable cell proliferation (cell hyperproliferation), a cellular hyperproliferative disorder, neoplasia, tumor or cancer, or metastasis. Particular therapies, treatment regimens, agents, drugs, protocol or processes can inhibit, decrease, slow, reduce, delay, or prevent cell proliferation, cell growth, cellular hyperproliferation, neoplastic, tumor, or cancer (malignant) growth, proliferation, survival or metastasis. Such treatments, therapies, regimens, protocols, agents and drugs, can operate by disrupting, reducing, inhibiting or delaying cell cycle progression or cell proliferation or growth; increasing, stimulating or enhancing cell apoptosis, lysis or death; inhibiting nucleic acid or protein synthesis or metabolism; reducing, decreasing, inhibiting or delaying cell division; or decreasing, reducing or inhibiting cell survival, or production or utilization of a cell survival factor, growth factor or signaling pathway (extracellular or intracellular).

Examples of anti-cell proliferative treatments and therapies include chemotherapy, immunotherapy, radiotherapy (ionizing or chemical), local or regional thermal (hyperthermia) therapy and surgical resection.

Specific non-limiting classes of anti-cell proliferative agents and drugs include alkylating agents, anti-metabolites, plant extracts, plant alkaloids, nitrosoureas, hormones (steroids), nucleoside and nucleotide analogues. Specific non-limiting examples of microbial toxins include bacterial cholera toxin, pertussis toxin, anthrax toxin, diphtheria toxin, and plant toxin ricin. Specific examples of drugs include cyclophosphamide, azathioprine, cyclosporin A, melphalan, chlorambucil, mechlorethamine, busulphan, methotrexate, 6-mercaptopurine, thioguanine, 5-fluorouracil, 5-fluorouridine, cytosine arabinoside, AZT, 5-azacytidine (5-AZC) and 5-azacytidine related compounds, bleomycin, actinomycin D, mithramycin, mitomycin C, carmustine, calicheamicin, lomustine, semustine, streptozotocin, teniposide, etoposide, hydroxyurea, cisplatin, carboplatin, levamisole, mitotane, procarbazine, dacarbazine, taxol, vinblastine, vincristine, vindesine, doxorubicin, daunomycin and dibromomannitol. Specific non-limiting examples of hormones include prednisone, prednisolone, diethylstilbesterol, flutamide, leuprolide, and gonatrophin releasing hormone antagonists.

Radiotherapy includes internal or external delivery to a subject. For example, alpha, beta, gamma and X-rays can administered to the subject externally without the subject internalizing or otherwise physically contacting the radioisotope. Specific examples of X-ray dosages range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease. Specific non-limiting examples of radionuclides include, for example, ⁴⁷Sc ⁶⁷Cu, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At, ²¹²Bi, ²¹³Bi, ²¹²Pb, ²²³Ra, ²²⁵Ac, ²²⁷Ac, and ²²⁸Th.

Antibodies that bind to tumor cells are a particular example of an anti-cell proliferative treatment or therapy. Anti-tumor antibodies include, for example, M195 antibody which binds to leukemia cell CD33 antigen (U.S. Pat. No. 6,599,505); monoclonal antibody DS6 which binds to ovarian carcinoma CA6 tumor-associated antigen (U.S. Pat. No. 6,596,503); human IBD12 monoclonal antibody which binds to epithelial cell surface H antigen (U.S. Pat. No. 4,814,275); and BR96 antibody which binds to Le^{x} carbohydrate epitope expressed by colon, breast, ovary, and lung carcinomas. Additional anti-tumor antibodies that can be employed include, for example, Herceptin (anti-Her-2 neu antibody), Rituxan®, Zevalin, Bevacizumab (Avastin), Bexxar, Campath®, Oncolym, 17-1A (Edrecolomab), 3F8 (anti-neuroblastoma antibody), MDX-CTLA4, IMC-C225 (Cetuximab) and Mylotarg.

As used here, the term “immune enhancing,” when used in reference to a treatment, therapy, agent or drug means that the treatment, therapy, agent or drug provides an increase, stimulation, induction or promotion of an immune response, humoral or cell-mediated. Such therapies can enhance immune response generally, or enhance immune response to a specific target, e.g., a cell proliferative or cellular hyperproliferative disorder such as a neoplasia, tumor or cancer, or metastasis.

Specific non-limiting examples of immune enhancing agents include antibody, cell growth factors, cell survival factors, cell differentiative factors, cytokines and chemokines. Additional examples of immune enhancing agents and treatments include immune cells such as lymphocytes, plasma cells, macrophages, dendritic cells, NK cells and B-cells that either express antibody against the cell proliferative disorder or otherwise are likely to mount an immune response against the cell proliferative disorder. Cytokines that enhance or stimulate immunogenicity include IL-2, IL-1α, IL-1β, IL-3, IL-6, IL-7, granulocyte-macrophage-colony stimulating factor (GMCSF), IFN-γ, IL-12, TNF-α, and TNFβ, which are also non-limiting examples of immune enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, PARC, TARC, LARC/MIP-3α, CKβ, CKβ6, CKβ7, CKβ8, CKβ9, CKβ11, CKβ12, C10, IL-8, ENA-78, GROα, GROβ, GCP-2, PBP/CTAPIIIβ-TG/NAP-2, Mig, PBSF/SDF-1, and lymphotactin are further non-limiting examples of immune enhancing agents.

Methods of the invention also include, among other things, methods that result in a reduced need or use of another treatment protocol or therapeutic regimen, process or remedy. For example, for a neoplasia, tumor or cancer, or metastasis, a method of the invention has a therapeutic benefit if in a given subject it results in a less frequent or reduced dose or elimination of an anti-cell proliferative (e.g., anti-neoplastic, anti-tumor or anti-cancer) or immune enhancing treatment or therapy, such as a chemotherapeutic drug, radiotherapy, immunotherapy, or surgery for neoplasia, tumor or cancer, or metastasis treatment or therapy.

In accordance with the invention, methods of reducing need or use of an anti-cell proliferative (e.g., anti-neoplastic, anti-tumor, anti-cancer or anti-metastasis) treatment or therapy are provided. In various embodiments, a method includes administering to a subject BARB4 antibody, as represented by antibody produced by hybridoma DSMZ Deposit No. DSM ACC2876, or heavy and light chain sequences set forth as SEQ ID NOs:1, 3, 5, 7; and 9, respectively, or antigen (e.g., a BARB4 target such as a cell membrane bound isoform of TAF 15 polypeptide), in an amount effective to treat a cellular hyperproliferative disorder (e.g., a neoplasia, tumor or cancer, or metastasis), and to reduce or eliminate need for an anti-cell proliferative (anti-neoplasia, anti-tumor or anti-cancer, or anti-metastasis) or immune-enhancing therapy. The methods can be performed prior to, substantially contemporaneously with or following administration of an anti-neoplastic, tumor, cancer or metastasis, or immune-enhancing therapy.

The doses or “amount effective” or “amount sufficient” in a method of treatment or therapy in which it is desired to achieve a therapeutic benefit or improvement includes, for example, any objective or subjective alleviation or amelioration of one, several or all pathologies, adverse symptoms or complications associated with or caused by the target (e.g., cellular hyperproliferative disorder), to a measurable or detectable extent, although preventing, inhibiting or delaying a progression or worsening of the target (e.g., cellular hyperproliferative disorder) pathology, adverse symptom or complication, is a satisfactory outcome. Thus, in the case of a cellular hyperproliferative disorder, the amount will be sufficient to provide a therapeutic benefit to a given subject or to alleviate or ameliorate a pathology, adverse symptom or complication of the disorder in a given subject. The dose may be proportionally increased or reduced as indicated by the status of treatment or therapeutic target (e.g., cellular hyperproliferative disorder) or any side effect(s) of the treatment or therapy.

Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. Additional exemplary non-limiting amounts (doses) range from about 0.5-50 mg/kg, 1.0-25 mg/kg, 1.0-10 mg/kg, and any numerical value or range or value within such ranges.

Methods of the invention may be practiced one or more times (e.g., 1-10, 1-5 or 1-3 times) per day, week, month, or year. The skilled artisan will know when it is appropriate to delay or discontinue administration. An exemplary non-limiting dosage schedule is 1-7 times per week, for 1, 2, 3, 4, 5, 6, 7, 8, 9, 10, 15, 20 or more weeks, and any numerical value or range or value within such ranges.

Of course, as is typical for any treatment or therapy, different subjects will exhibit different responses to treatment and some may not respond or respond inadequately to a particular treatment protocol, regimen or process. Amounts effective or sufficient will therefore depend at least in part upon the disorder treated (e.g., cell proliferation, benign hyperplasia or a neoplasia, tumor or cancer and the type or stage, e.g., the tumor or cancer grade and if it is advanced, late or early stage), the therapeutic effect desired, as well as the individual subject (e.g., the bioavailability within the subject, gender, age, etc.) and the subject's response to the treatment based upon genetic and epigenetic variability (e.g., pharmacogenomics).

Cell toxicity and viability (cell apoptosis, lysis, growth proliferation, etc.) can be measured in a variety of ways on the basis of calorimetric, luminescent, radiometric, or fluorometric assays known in the art. Colorimetric techniques, for example, Trypan Blue exclusion can be used to determine cell viability. In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, for example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochrome-labeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, for example, [³H]-Thymidine, which is incorporated into newly synthesized DNA of living cells and frequently used to determine proliferation of cells. Chromium (⁵¹Cr)—release from dead cells can be quantitated by scintillation counting in order to quantitate cell viability.

Luminescent techniques for determining cell viability include, for example, the CellTiter-Glo luminescent cell viability assay (Promega Madison Wis.). This technique quantifies the amount of ATP present to determine the number of viable cells.

Commercially available kits for determining cell viability and cell proliferation include, for example, Cell Proliferation Biotrak ELISA (Amersham Biosciences Piscataway, N.J.); the Guava ViaCount™ Assay, which provides rapid cell counts and viability determination based on differential uptake of fluorescent reagents (Guava Technologies, Hayward, Calif.); the CyQUANT® Cell Proliferation Assay Kit (Molecular Probes, Inc., Eugene, Oreg.); and the CytoLux Assay Kit (PerkinElmer Life Sciences Inc., Boston, Mass.). The DELFIA® Assay Kits (PerkinElmer Life Sciences Inc., Boston, Mass.) can determine cell proliferation and viability using a time-resolved fluorometric method. The Quantos™ Cell Proliferation Assay is a fluorescence-based assay that measures the fluorescence of a DNA-dye complex from lysed cells (Stratagene, La Jolla, Calif.). The CellTiter-Glo cell viability assay is a luminescent assay for measuring cell viability (Promega, Madison Wis.).

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig). Subjects include disease model animals (e.g., such as mice, rats and non-human primates) for studying in vivo efficacy (e.g., a neoplasia, tumor or cancer, or metastasis animal model). Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects include mammals (e.g., humans) in need of treatment, that is, they have undesirable or aberrant cell proliferation (cell hyperproliferation) or a cellular hyperproliferative disorder. Subjects also include those at risk of having a undesirable cell proliferation or a cellular hyperproliferative disorder. Subjects further include a subject in need of an anti-cell proliferative or immune enhancing treatment or therapy due to a lab or clinical diagnosis warranting such treatment, subjects undergoing an anti-cell proliferative or immune enhancing therapy, and subjects having undergone an anti-cell proliferative or immune enhancing therapy and are at risk of relapse or recurrence.

At risk subjects include those with a family history, genetic predisposition, or who have suffered a previous affliction with a cell proliferative or cellular hyperproliferative disorder (e.g., a benign hyperplasia, neoplasia, tumor or cancer, or metastasis), and are at risk of relapse or recurrence. At risk subjects further include environmental exposure to carcinogens or mutagens, such as smokers, or those in an occupational (industrial, chemical, agricultural) setting. Such subjects at risk for developing a cell proliferative or cellular hyperproliferative disorder such as neoplasia, tumor or cancer can be identified with genetic screens for tumor associated genes, gene deletions or gene mutations. Subjects that lack Brca1 are at risk for developing breast cancer, for example. Subjects at risk for developing colon cancer have deleted or mutated tumor suppressor genes, such as adenomatous polyposis coli (APC), for example. At risk subjects having particular genetic predisposition towards cell proliferative disorders are known (see, e.g., The Genetic Basis of Human Cancer 2^{nd} ed. by Bert Vogelstein (Editor), Kenneth W. Kinzler (Editor) (2002) McGraw-Hill Professional; The Molecular Basis of Human Cancer. Edited by W B Coleman and G J Tsongalis (2001) Humana Press; and The Molecular Basis of Cancer. Mendelsohn et al., WB Saunders (1995)).

At risk subjects can therefore be treated in order to inhibit or reduce the likelihood of developing a cell proliferative or cellular hyperproliferative disorder, or after having been cured of a cell proliferative disorder, suffering a relapse or recurrence of the same or a different cell proliferative or cellular hyperproliferative disorder. The result of such treatment can be to reduce the risk of developing a cell proliferative or cellular hyperproliferative disorder, or to prevent a cell proliferative or cellular hyperproliferative disorder, or a pathology, adverse symptom or complication thereof in the treated at risk subject.

The invention further provides kits, including antigens, antibodies, functional fragments, modified and variants forms, nucleic acids, agents, drugs and pharmaceutical formulations, packaged into suitable packaging material, optionally in combination with instructions for using the kit components, e.g. instructions for performing a method of the invention. In various embodiments, a kit includes an antigen (e.g., a BARB4 target such as a cell membrane bound isoform of TAF 15 polypeptide), or an antibody such as BARB4 antibody, as represented by antibody produced by hybridoma DSMZ Deposit No. DSM ACC2876, or heavy and light chain sequences set forth as SEQ ID NOs:1, 3, 5, 7; and 9, respectively. In one aspect, the instructions are for treating undesirable cell proliferation or hyperproliferation, or a cellular hyperproliferative disorder. In another aspect, the instructions are for treating a neoplasia, tumor or cancer, or metastasis. In a further embodiment, a kit includes a BARB4 antibody, as represented by antibody produced by hybridoma DSMZ Deposit No. DSM ACC2876, or heavy and light chain sequences set forth as SEQ ID NOs:1, 3, 5, 7; and 9, respectively, and instructions for treating undesirable cell proliferation or hyperproliferation, or a cellular hyperproliferative disorder, and an anti-cell proliferative or immune enhancing treatment, agent or drug. In various aspects, a kit includes an anti-neoplastic, anti-cancer or anti-tumor agent. In still a further aspects, a kit includes an article of manufacture, for example, an article of manufacture for delivering the antibody or nucleic acid, anti-cell proliferative or immune enhancing treatment, agent or drug into a subject locally, regionally or systemically.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a cell proliferative or cellular hyperproliferative disorder, an assay for screening for, detecting or identifying an antigen (e.g., a BARB4 target such as a cell membrane bound isoform of TAF 15 polypeptide) or a cell to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, invention pharmaceutical compositions can be included in a container, pack, or dispenser together with instructions for administration to a subject to treat a cell proliferative or cellular hyperproliferative disorder, such as a neoplasia, tumor or cancer, or metastasis. Instructions may additionally include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include control components for assaying for activity, e.g., a control sample or a standard. Each component of the kit can be enclosed within an individual container or in a mixture and all of the various containers can be within single or multiple packages.

Antigens (e.g., a BARB4 target such as a cell membrane bound isoform of TAF 15 polypeptide), antibodies (e.g., BARB4 antibody, as represented by antibody produced by hybridoma DSMZ Deposit No. DSM ACC2876, or heavy and light chain sequences set forth as SEQ ID NOs:1, 3, 5, 7; and 9, respectively, nucleic acids, and other compositions and methods of the invention can be included in or employ pharmaceutical formulations. Such pharmaceutical formulations are useful for treatment of, or administration or delivery to, a subject in vivo or ex vivo.

Pharmaceutical formulations include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the formulations.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intrarterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

Solutions or suspensions used for parenteral application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate or gelatin can prolong absorption of injectable compositions.

Sterile injectable formulations can be prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

The pharmaceutical formulations can be prepared with carriers that protect against rapid elimination from the body, such as a controlled release formulation or a time delay material such as glyceryl monostearate or glyceryl stearate. The formulations can also be delivered using articles of manufacture such as implants and microencapsulated delivery systems to achieve local, regional or systemic delivery or controlled or sustained release.

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation (Palo Alto, Calif.). Liposomal suspensions (including liposomes targeted to cells or tissues using antibodies or viral coat proteins) can also be used as pharmaceutically acceptable carriers. These can be prepared according to known methods, for example, as described in U.S. Pat. No. 4,522,811.

Additional pharmaceutical formulations appropriate for administration are known in the art (see, e.g., Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Remington's Pharmaceutical Principles of Solid Dosage Forms, Technonic Publishing Co., Inc., Lancaster, Pa., (1993)).

The compositions used in accordance with the invention, including proteins (antibodies), nucleic acid (inhibitory), treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides cell-free (e.g., in solution, in solid phase) and cell-based (e.g., in vitro or in vivo) methods of screening, detecting and identifying a cell or antigen (e.g., a BARB4 target such as a cell membrane bound isoform of TAF 15 polypeptide) to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. The methods can be performed in solution, in vitro using a biological material or sample, and in vivo, for example, using neoplastic, tumor or cancer, or metastasis cells, tissue or organ (e.g., a biopsy) from an animal.

In accordance with the invention, there are provided methods of identifying, detecting or screening for antigen (e.g., a BARB4 target, such as a sequence with identity to TAF 15 polypeptide, for example, a sequence identical to 10, 20, 30, 40, 50, 60, 70, 80, 90 or 100 or more (e.g., full length) contiguous amino acids set forth in SEQ ID NO:11 or 12, a cell membrane bound isoform of TAF 15 polypeptide, or a TAF 15 polypeptide that includes a carbohydrate moiety), or a cell or antigen to which BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds. In one embodiment, a method includes contacting a biological material or sample with an antibody or functional fragment thereof under conditions allowing binding of antibody to the antigen having sequence identity to TAF 15 polypeptide; and assaying for binding of the antibody to the antigen. Binding of the antibody to the antigen (e.g., a BARB4 target such as a cell membrane bound isoform of TAF 15 polypeptide) detects the presence of the antigen having sequence identity to TAF 15 polypeptide. In another embodiment, a method includes contacting a biological material or sample with a BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds under conditions allowing binding of the antibody to a cell or antigen (e.g., a BARB4 target such as a cell membrane bound isoform of TAF 15 polypeptide); and assaying for binding of the antibody to the cell or antigen. The binding of the antibody to a cell or antigen (e.g., a BARB4 target such as a cell membrane bound isoform of TAF 15 polypeptide) detects their presence. In one aspect, the biological material or sample is obtained from a mammalian subject. In a further aspect, the antibody that binds to the cell or antigen (e.g., a BARB4 target such as a cell membrane bound isoform of TAF 15 polypeptide) is distinct from BARB4 antibody, as represented by antibody produced by hybridoma DSMZ Deposit No. DSM ACC2876, or heavy and light chain sequences set forth as SEQ ID NOs:1, 3, 5, 7; and 9, respectively, binds.

The invention also provides cell-free (e.g., in solution, in solid phase) and cell-based (e.g., in vitro or in vivo) methods of diagnosing a subject having or at increased risk of having undesirable or aberrant cell proliferation or a cellular hyperproliferative disorder (e.g., neoplasia, tumor or cancer, or metastasis). The methods can be performed in solution, in vitro using a biological material or sample, for example, a biopsy of suspicious cells that may comprise or be indicative of neoplastic, tumor or cancer, or metastasis cells, tissue or organ. The methods can also be preformed in vivo, for example, in an animal.

In accordance with the invention, there are provided methods of diagnosing a subject having or at increased risk of having undesirable or aberrant cell proliferation or a cellular hyperproliferative disorder (e.g., neoplasia, tumor or cancer, or metastasis). In one embodiment, a method includes providing a biological material or sample from a subject, contacting the biological material or sample with an antibody or functional fragment thereof under conditions allowing binding of antibody to the antigen having sequence identity to TAF 15 polypeptide; and assaying for binding of the antibody to the antigen. Binding of the antibody to the antigen (e.g., a BARB4 target such as a cell membrane bound isoform of TAF 15 polypeptide) diagnoses the subject as having or at increased risk of having undesirable or aberrant cell proliferation or a cellular hyperproliferative disorder (e.g., neoplasia, tumor or cancer, or metastasis). In another embodiment, a method includes providing a biological material or sample from a subject, contacting the biological material or sample with a BARB4 antibody, as represented by antibody produced by hybridoma DSMZ Deposit No. DSM ACC2876, or heavy and light chain sequences set forth as SEQ ID NOs:1, 3, 5, 7; and 9, respectively, under conditions allowing binding of the antibody to a cell or antigen; and assaying for binding of the antibody to the cell or antigen. Binding of the antibody to the cell or antigen diagnoses the subject as having or at increased risk of having undesirable or aberrant cell proliferation or a cellular hyperproliferative disorder (e.g., neoplasia, tumor or cancer, or metastasis). In one aspect, the biological material or sample is obtained from a human. In another aspect, the biological material or sample comprises a biopsy (e.g., a biopsy of lung, pancreas, stomach, breast, esophagus, ovary or uterus).

Identifying, detecting, screening and diagnostic assays of the invention can be practiced by analysis of suspect hyperproliferating cells, for example, a cell of a cellular hyperproliferative disorder. Cells include hyperproliferating, immortalized, neoplastic, tumor and cancer cell lines and primary isolates derived from breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, cervix, bladder, testicle, penis, prostate), kidney, pancreas, adrenal gland, liver, bone, bone marrow, lymph, blood, muscle, skin, and the hematopoetic system, and metastasis or secondary sites.

The term “contacting,” when used in reference to a composition such as a protein (e.g., antibody), material, sample, or treatment, means a direct or indirect interaction between the composition (e.g., protein such as an antibody) and the other referenced entity. A particular example of direct interaction is binding. A particular example of an indirect interaction is where the composition acts upon an intermediary molecule, which in turn acts upon the referenced entity. Thus, for example, contacting a cell (e.g., that comprises a cellular hyperproliferative disorder) or an antigen with an antibody includes allowing the antibody to bind to the cell or antigen, or allowing the antibody to act upon an intermediary (e.g., antigen) that in turn acts upon the cell or antigen.

The terms “assaying” and “measuring” and grammatical variations thereof are used interchangeably herein and refer to either qualitative or quantitative determinations, or both qualitative and quantitative determinations. When the terms are used in reference to binding, any means of assessing the relative amount, affinity or specificity of binding is contemplated, including the various methods set forth herein and known in the art. For example, antibody binding can be assayed or measured by an ELISA assay.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to an “antigen” or an “antibody” includes a plurality of antigens or antibodies, and reference to “a treatment or therapy” can include multiple simultaneous, consecutive or sequential treatments or therapies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes any numerical value or range within or encompassing such values, such as 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and any numerical range within such a range, such as 90-92%, 90-95%, 95-98%, 96-98%, 99-100%, etc. In an additional example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a range, such as 1-2, 5-10, 10-50, 50-100, 100-500, 100-1000, 500-1000, 1000-2000, 1000-5000, etc. In a further example, reference to a range of KD 10⁻⁵ M to about KD 10⁻¹³ M includes any numerical value or range within or encompassing such values.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes various exemplary materials and methods and data.

### Immunohistochemistry (IHC) Analysis

Immunohistochemistry with human IgG on human tissue naturally results in high background staining. Accordingly, BARB4 was labeled with biotin to reduce background.

BARB4 antibody and an unrelated commercial IgG control (Chrompure IgG, Dianova) where labelled with “EZ-Link Maleimide-PEO Solid Phase Biotinylation Kit” from Pierce (Cat-# 21930) and Immunohistochemistry was initiated on different tumor tissue. Parrafin embedded tissue was first deparaffinized by treatment with Xylene 1 for 5 min, Xylene 2 for 5 min, followed by 100% Ethanol 1 for 5 min, 100% Ethanol 2 for 5 min, 70 ml Methanol+500 μl H2O2 for 7 min, 90% Ethanol 1 for 3 min, 90% Ethanol 2 for 3 min, 80% Ethanol 1 for 3 min, 80% Ethanol 2 for 3 min, 70% Ethanol 1 for 3 min, 70% Ethanol 2 for 3 min, and then washed three times with distilled H2O. (1 and 2 mean that the tissues were treated 2 times with the same alcohol, one after the another, in two different tanks.) Tissue was then heated in Citric acid, pH 5,5 in a water-bath at 99.9° C. for 20 min, placed in Tris/NaCl (Tris, 0.6 g/l and NaCl 8.1 g/l, pH 7.4) for 5 min, block with 0.5% BSA in PBS for 60 min, and then washed three times with Tris/NaCl (Tris, 0.6 g/l and NaCl 8.1 g/l, pH 7.4). Primary antibody (150 μl per microscope slide) was added, or a negative control biotinylated isotype matched control human antibody (Chrompure IgG, Dianova, Germany) or a positive controlanti-cytokeratin antibody, and incubated for 30 min in a humidified chamber at 37° C. Tissue was washed three times with Tris/NaCl (Tris, 0.6 g/l and NaCl 8.1 g/l, pH 7.4). Secondary antibody: (150 g per microscope slide) was subsequently added and incubated for 30 min in a humidified chamber at room temperature (for biotinylated antibodies: NeurtrAvidin 1:100 in PBS), washed three times with Tris/NaCl (Tris, 0.6 g/l and NaCl 8.1 g/l, pH7.4), placed in PBS for 10 min. Tiusse was subsequently incubated with diaminobenzidine (0.05%)-hydrogen peroxide (0.02%) (150 μl per microscope slide) (Sigma, Taufkirchen, München, Germany) for 10 min, washed three times with H2O, washed once with distilled H2O, incubated with hematoxylin/eosin for 5 min, and placed under running tap water 10-15 min. Tissue was then washed again with distilled H2O, and the slides covered with Aquatex (Merck Bioscience). The data is illustrated in Table 1 below:

TABLE 1
IHC Data

| | Tissue | Carcinoma Type | Barb 4 +/− |
|---|---|---|---|
| Malignant | Stomach | Adeno (intestinal) | 2/0 |
| Tissues | | Adeno (Cardio, intestinal) | 1/0 |
| | Lung | Carcinoid (neuron-endocrine) | 1/0 |
| | | Squamous cell | 2/0 |
| | | Lymph Node Metastasis | 1/0 |
| | Colon | Adeno | 1/0 |
| | | Liver Metastasis | 2/0 |
| | Pancreas | Adeno | 0/1 |
| | | Lymph Node Metastasis | 4/0 |
| | Esophagus | Adeno (Barrett Carcinoma) | 1/0 |
| | | Squamous cell | 1/0 |
| | Prostrate | Adeno | 1/0 |
| | Breast | Invasive ductal | 2/0 |
| | | Lung Metastasis | 1/0 |
| | | Lymph Node Metastasis | 1/0 |
| | Melanoma | Metastases of malignant melanoma | 5/0 |
| | Tissue | Cell Type | Barb 4 +/− |
| Healthy | Lung | glandular, alveolar | 0/3 |
| Tissues | Breast | glandular | 0/2 |
| | Colon | glandular | 0/3 |
| | Stomach | glandular | 0/3 |
| | Pancreas | glandular | 0/2 |
| | Esophagus | epithelial | 0/1 |

### FACs Analysis

To analyze tumor cell binding, pancreas cancer cells (BXPC-3), stomach cancer cells (23132/87), lung carcinoma cells (A549) and malignant melanoma cells (HTB-69 and CRL-1424) were grown to subconfluency in complete medium, detached with Trypsin/EDTA and incubated on ice for 1 h for recreation. Tumor cells were subsequently incubated on ice with BARB4 antibody (diluted in PBS containing 0.01% sodium azide), or human isotype-matched control antibody (Chrompure human IgG, Dianova, Hamburg, Germany) for 20 min on ice. Following incubation, cells were incubated with a FITC-labeled rabbit anti-human IgG antibody (1:50, Dako, Germany) for 20 min on ice, and cells were analyzed by flow cytometry (FACScan; Becton Dickinson, USA). The data indicates that BARB4 antibody binds to pancreas (BXPC-3) cancer cells, stomach cancer cells (23132/87), lung carcinoma cells (A549) and malignant melanoma cells (HTB-69 and CRL-1424).

### MTT Cell Proliferation Assay

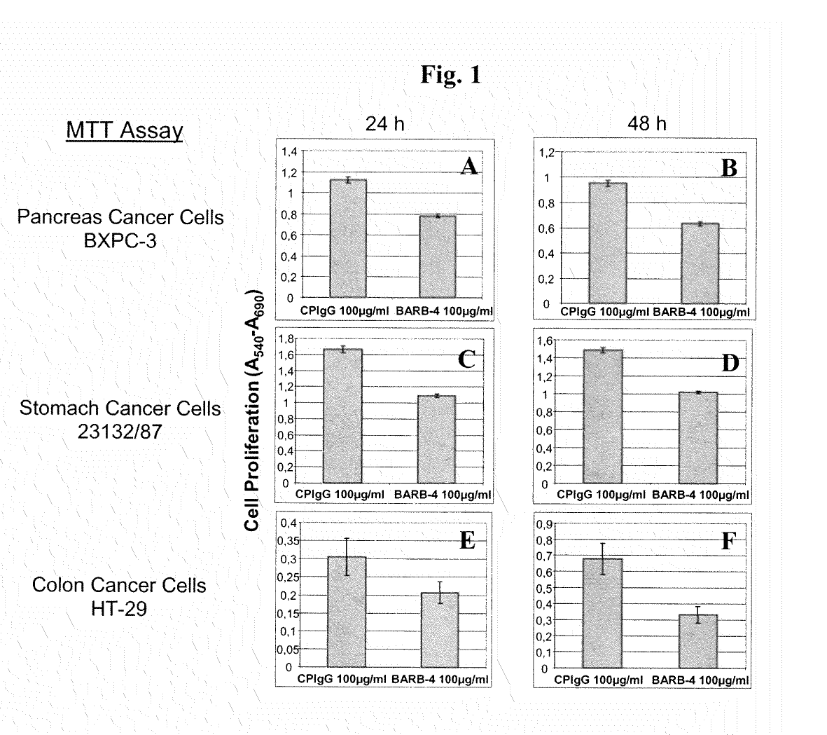

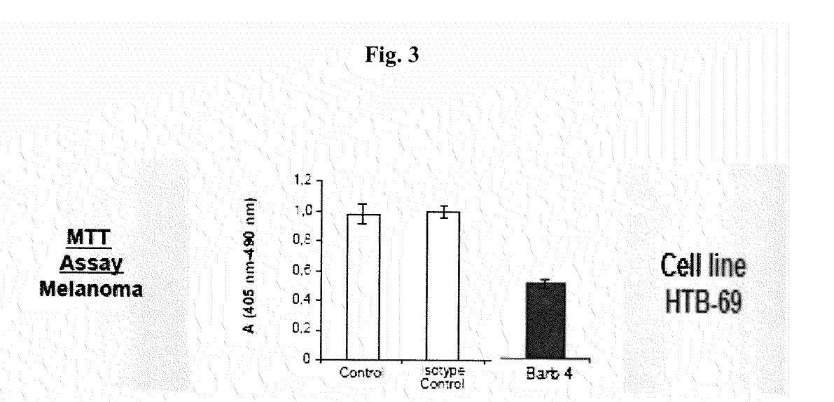

To analyze tumor cell proliferation, stomach cancer cells (23132/87), malignant melanoma cells (HTB-69), colon and pancreas cancer cells were trypsinized and resuspended in 10 ml of RPMI-1460 medium that contained 10% Fetal Calf Serum (FCS), 1% glutamine, and 1% penicillin/streptomycin (complete medium). Cells (1×10⁴) were plated into 96well plates (24 h) and incubated with purified antibody or supernatants containing antibody for 48 h at 37° C. After incubation, 50 μl MTT (3-(4,5-dimethylthiazol-2-yl)-2,5-diphenyltetrazolium bromide) solution (5 mg/ml in PBS), (Sigma, Taufkirchen, München, Germany) was added to each well. The 96-well plate was incubated for 30 minutes at 37° C. and centrifuged for 10 minutes at 2800 rpm, the supernatant aspirated, 150 μl of DMSO added to each well, and the cell pellet was resuspended. Absorption was determined at a wavelength of 540 nm and at a reference wavelength of 690 nm in an ELISA reader. The data is illustrated in FIGS. 1A-1F and 3.

BARB4 was also evaluated for inhibition of proliferation of other cancer cell lines, including BxPC-3, COLO 205, H460, MDA-MB-231 and MIAPaCa-2 cells, as well as HT-29, HCT-116, BxPC-3, PANC-1, A549, PC-3 and HCT116 cells. In brief, various human cancer cell lines obtained from the American Type Culture Collection (ATCC) were established using standard in vitro culture methods and ATCC recommended media in 175 cm2 BD tissue culture treated flasks. In particular, MDAMB-231 cultures were incubated in a humidified 37° C., 100% air incubator, and BxPC-3, COLO 205, H460 and MIAPaCa-2 cultures were incubated in a humidified 37° C., 5% CO2, 95% air incubator. Cultures were sub-cultivated regularly to maintain log phase growth. All cell lines were brought up from cryopreservation using the original recommended ATCC media type and supplements. Cell line specific seeding densities were based on historical data from MIR Preclinical Services for growth over a 72 hr period.

On the day of IC50 plate seeding, the adherent cells were removed from the flasks for each cell line using trypsin EDTA, 1× (Cell Gro #25-053-CI). Trypsin was deactivated using complete media and the cells were pooled (each line done separately). The pooled cells in complete media were counted using trypan blue exclusion with a Neubauer Bright-Line® hemacytometer. The percent viability for each cell line was as follows:

BxPC-3 (1)—99.7% viable
BxPC-3 (2, repeat)—98.1% viable
COLO 205-95.9% viable
H460-98.9% viable
MDA-MB-231—98.8% viable
MIAPaCa-2—92.0% viable

Based on live cell counts, each suspension was then diluted in complete media (containing required supplements) to achieve the desired seeding density for each cell line. Once seeded, the cells were allowed to attach to the plates overnight and then treated with BA4 or cisplatin 24 hr post-seeding.

BARB4 was stored in the Nalgene® cryogenic box. The BARB4 solution arrived in a sealed foam container with cold packs (temp. measured to be ˜10° C. on arrival) and was maintained at 4° C. during storage. BARB4 was a clear, colorless solution.

BARB4 was serially diluted 1:2 (2× final conc. in dilution reservoir=1600 μg/ml) in media to achieve a final 800 μg/ml starting concentration in the top dosage wells of the treatment plate by diluting 1:2 when transferred from the reservoir to the treatment plate. Antibody was added to columns 1 through 10 of the treatment plate. Column 11 was for cells+media only control and column 12 was for a media only blank. Cisplatin (positive control) was manufactured by Sigma (# M4394, lot 087K1349) and supplied as a fine, dark yellow powder, in an amber vial, stored at room temperature and housed in a covered box to prevent exposure to light. Cisplatin was previously dissolved in 0.9% saline to make a clear, colorless 4 mM stock solution, aliquoted and frozen at −20° C. Frozen aliquots were quickly thawed by holding the tubes in hand, placed on wet ice and diluted (2× conc. dilution reservoir) to achieve a final 1 mM starting concentration in the top dosage wells of the treatment plate (diluted 1:2 when transferred from the dilution reservoir to the treatment plate). Serial dilutions of 1:4 were made from wells 1 through 10 of the dilution reservoir prior to transfer to the treatment plate.

The BARB4 stock solution (10.5 mg/ml) was prepared by dilution in complete media according to the table below for a final starting treatment concentration of 800 μg/ml. The 4 mM stock solution of cisplatin in 0.9% saline was diluted in complete media for a final starting treatment concentration of 1 mM.

Cells were exposed to BARB4 or cisplatin for a 24 hr period. The plates were incubated overnight at 37° C. At 24 hr and 48 hr post-treatment, the MTT assay was performed as described below.

The anti-proliferative activity of BARB4 and cisplatin was evaluated using the MTT cell proliferation assay (ATCC catalog #30-1010K). The assay is based on the reduction of yellow tetrazolium MTT (3-(4,5-dimethylthiazolyl-2)-2,5-diphenyltetrazolium bromide) by metabolically active cells forming purple formazan crystals. The purple formazan is solubilzed with detergent and quantified spectrophotometrically at 570 nm (MTT Cell Proliferation Assay, ATCC 30-1010K; Van de Loosdrecht, et al. J. Immunol. Methods 174:311 (1994): Ferrari, et al. J. Immunol. Methods 131:165 (1990); Gerlier, D., and N. Thomasset. J. Immunol. Methods 94:57 (1986); Alley, et al. Cancer Res. 48:589 (1988); and Mosmann, T. J. Immunol. Methods 65:55 (1983)). The MTT Cell Proliferation Assay Kit from ATCC contains ready to use MTT and detergent solutions.

Cells in the log phase of growth were seeded into 96-well culture plates in 0.1 mL of complete medium in all wells except column 12 which was reserved for media-only control (blank) and allowed to attach overnight at 37° C. Compounds were diluted in complete culture media and added to each well in a volume of 0.01 mL for a final 1:2 dilution from the 2× dilution reservoir concentration. At 24 and 48 hr post-treatment, 100 μl of media was removed and 0.01 mL of MTT reagent was added to each well. The plates were returned to the incubator for four hours. Detergent reagent (0.1 mL) was then added and the plates incubated overnight at 37° C. in the dark to solubilize the formazan crystals. The absorbance at 570 nm was measured with a SpectraMAX® Plus plate reader (Molecular Devices Corporation) at 24 and 48 hr post-detergent addition. Absorbance values were converted to percent of control and then plotted against test agent concentrations for IC50 calculations using SoftMax® Pro v. 5.2 (Molecular Devices Corporation). Plots of percent of control vs. compound (BARB4 or cisplatin) concentration were analyzed using the 4 parameter equation to obtain IC50 values and other parameters that describe the sigmoidal curve along with an R2 value.

The experimentally determined IC50 values for BARB4 (μg/ml) and cisplatin (μM) against BxPC-3, COLO 205, H460, MDA-MB-231 and MIAPaCa-2 cell lines are summarized in Table 2. The BARB4 antibody did not exhibit a complete range of inhibition from 0 to 100%. Therefore, these “IC50” values are really “EC50” values (Effective Concentration), which refers to the concentration where 50% inhibition is seen for the total observed effect.

TABLE 2

| | | | Cisplatin | Cisplatin |
|---|---|---|---|---|
| Cell Line | BA4 (24 hr) | BA4 (48 hr) | (24 hr) | (48 hr) |
| BxPC-3 | >800 μg/ml | >800 μg/ml | 39.2 | 9.41 |
| BxPC-3 repeat | >800 μg/ml | >800 μg/ml | 13.6 | 5.65 |
| COLO 205 | 66.8 | 2.78 | 58.5 | 62.9 |
| H460 | 413 | >800 μg/ml | 63.3 | 6.37 |
| MDA-MB-231 | 154 | 193 | 97.5 | 233 |
| MIAPaCa-2 | 34.9 | 21.1 | 43.9 | 29.8 |

IC50 values for BARB44 against *BXPC-3 did not correlate with those from a previous study that used the same compound, treatment concentrations and duration of exposure. This study was repeated for the BxPC-3 line, “repeat”) and yielded the same results.

The following is the cell culture protocol for passaging adherent cells: All manipulations were carried out in a Class II HEPA filtered biosafety hood using sterile technique.

1. Aspirated and discarded culture medium.

2. Added 3 mL of 0.25% (w/v) Trypsin, 0.53 mM EDTA solution (CellGro 25-053-CI) to each flask to ensure complete coverage of the cell monolayer by rocking gently in multiple directions. Removed 2 ml of the Trypsin.

3. Observe cells under an inverted microscope until the cell layer was dispersed (3-5 minutes). Flasks were incubated at 37° C. to facilitate dispersal, if necessary.

4. Added extra volume of fresh media to neutralize the trypsin. Aspirated the cells by gently pipetting and rinsing the monolayer area then immediately added appropriate aliquots of the cell suspension to new culture vessels (T175 flasks) containing 30 mL fresh pre-warmed (room temperature to 37° C.) media.

5. Incubated cultures at 37° C. in a humidified 100% air or 5% CO2 incubator, and subculture and/or change media every 2-3 days.

The following were the cell line propagation conditions (Supplement percentages are vol./vol.):

### Cell Line: BxPC-3 (Adherent)

### Media: RPMI1640 (CellGro 10-040-CV)

### Supplements: 1% (1M HEPES)+1% NaPyruvate+1% (45% Glucose), 10% FBS, 1% PSG

### Atmosphere: 5% CO2, 95% air

### Cell Line: COLO 205 (Adherent)

### Media: RPMI1640 (CellGro 10-040-CV)

### Supplements: 1% (1M HEPES)+1% NaPyruvate+1% (45% Glucose), 10% FBS, 1% PSG

### Atmosphere: 5% CO2, 95% air

### Cell Line: H460 (Adherent)

### Media: RPMI 1640 (CellGro 10-040-CV)

### Supplements: 1% (1M HEPES)+1% NaPyruvate+1% (45% Glucose), 10% FBS, 1% PSG

### Atmosphere: 5% CO2, 95% air

### Cell Line: MDA-MB-231 (Adherent)

Media. L-15 (CellGro 10-045-CV)

### Supplements: 10% FBS, 1% PSG Atmosphere. 100% air

### Cell Line: MIAPaCa-2 (Adherent)

### Media: DMEM (CellGro 10-013-CV)

### Supplements: 10% FBS, 2.5% HS, 1% PSG

### Atmosphere: 5% CO2, 95% air

### Media Supplements Used. * FBS—Fetal Bovine Serum (Gibco 10082-147; lot # 1354986), * PSG—Penicillin, Streptomycin, L-Glutamine Solution (CellGro 30-009-CI) and * HS—Horse Serum (ATCC #30-2041; lot # 3000412).

### Seeding Densities* for IC50 Assays: BxPC-3 12×10³; COLO 205 6.25×10³; H460 7.5×10³; MDA-MB-231 15×10³; and MIAPaCa-2 15×10³

Using the same MTT assay as described for the BxPC-3, COLO 205, H460, MDA-MB-231 and MIAPaCa-2 cells, proliferation of HT-29, HCT-116, BxPC-3, PANC-1, A549, PC-3 and HCT116 cells was evaluated and IC50 (μg/ml) was calculated at 24, 48 and 72 hours. The cell culture protocol for passaging adherent cells used is substantially as described above for the BxPC-3, COLO 205, H460, MDA-MB-231, and MIAPaCa-2 cells. The following were the cell line propagation conditions:

### Cell Line: A549 (Adherent)

Media. Ham's F-12 (CellGro 10-080-CV)

### Supplements: 10% FBS, 1% PSG

Atmosphere: 5% CO2, humidified

### Cell Line: PC-3 (Adherent)

### Media: Ham's F-12 (CellGro 10-080-CV)

### Supplements: 10% FBS, 1% PSG

Atmosphere: 5% CO2, humidified

### Cell Line: HT-29 (Adherent)

### Media: McCoy's 5a (CellGro 10-050-CV)

### Supplements: 10% FBS, 1% PSG

Atmosphere: 5% CO2, humidified

### Cell Line: HCT116 (Adherent)

### Media: McCoy's 5a (CellGro 10-050-CV)

### Supplements: 10% FBS, 1% PSG

Atmosphere: 5% CO2, humidified

### Cell Line: BxPC-3 (Adherent)

Media. RPMI 1640 (CellGro 10-040-CV)
Supplements: 10% FBS, 1% PSG, 1% Na Pyruvate, 1% HEPES (1 M stock), 1%
Glucose (45% stock)
Atmosphere: 5% CO2, humidified

### Cell Line: PANC-1 (Adherent)

### Media: DMEM (CellGro 10-013-CV)

### Supplements: 10% FBS, 1% PSG

Atmosphere. 5% CO2, humidified
Media Supplements Used: * FBS—Fetal Bovine Serum (Gibco 10082-147; lot #1354986), * PSG—Penicillin, Streptomycin, L-Glutamine Solution (CellGro 30-009-CI), * 0.1M Na Pyruvate (CellGro 25-000-CI), * 1.0M HEPES (CellGro 25-060-CI), AND * 45% Glucose (CellGro 25-037-CI).
Seeding Densities* for IC50 Assays: A549 2.5×10³; PC-3 6.0×10³; HT-29 12×10³; HCT-116 8.0×10³; BxPC-3 12×10³; and PANC-1 6.0×10³ cells.

As Illustrated in Table 3, BARB4 inhibited proliferation of HT-29, HCT-116, BxPC-3, PANC-1, A549, PC-3 and HCT 116 cell lines. The data demonstrates that BARB4 inhibits proliferation of many different types of cancer cells.

TABLE 3

| | IC₅₀(μg/mL) | | |
|---|---|---|---|
| | 24 hours | 48 hours | 72 hours |
| | BARB4 | BARB4 | BARB4 |
| A549 | 98.9 | 111.2 | 122.2 |
| PC-3 | 57.2 | 64.2 | 155.7 |
| HT-29 | 399.5 | 300.1 | 228.8 |
| HCT-116 | 24.8 | 77.3 | 95.2 |
| BxPC-3 | 88.9 | 362.8 | 511.4 |
| PANC-1 | 91.4 | 337.1 | 353.2 |

### Cell Death (Apoptosis) Assay

Cell Death Detection Elisaplus (Roche, Mannheim, Germany) was used to analyze the extent to which the antibodies induce apoptosis. This assay is based upon a quantitative sandwich-enzyme-immunoassay principle using mouse monoclonal antibodies directed against DNA and histones, respectively. This assay allows the specific determination of mono- and oligo-nucleosomes which are released into the cytoplasm of cells which die from apoptosis.

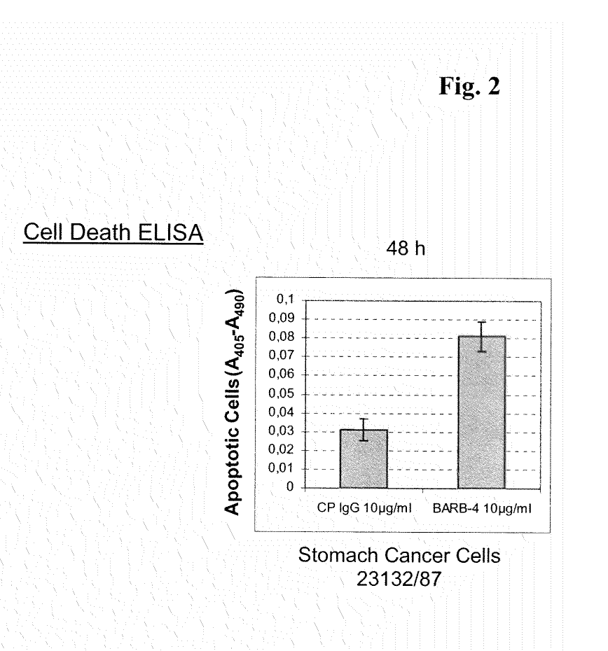

In brief, cells (1.0×10⁴) were plated in 96 well plates and incubated with antibody at 37° C. and 7% CO2 for 24/48 hours. After incubation, the cells were centrifuged at 200 g for 10 minutes, the supernatant was aspirated and 200 μl of lysis buffer added, which resulted in the lysis of cells following a 30 minute incubation at room temperature. After centrifuging again, 20 μl of the supernatant was added to streptavidin-coated micro-titer plates and 80 μl of the immuno-reagent (1/20 Anti-DNA-peroxidase (anti-DNA-POD) antibody, which reacts with the DNA components of the nucleosomes, 1/20 Anti-Histone Biotin, 18/20 incubation buffer) was added. A positive control and the blank included in the manufacturers test kit were used. After plates were incubated for 2 hours while being mixed at approximately 250 rpm, each well was washed three times with 250 μl of incubation buffer, followed by addition of 100 μl of ABTS solution (1 ABTS (2,2′-Azino-di[3-ethyl-benz-thiazolin-sulfonat) tablet in 5 ml substrate buffer) to each well. The plates were mixed again and intensity of antibody-induced apoptosis is reflected in the intensely green precipitate. The color intensity was determined using an ELISA reader at a wavelength of 415 nm against a reference wavelength of 490 nm. Based on this color intensity, the intensity of the antibody-induced apoptosis was calculated. The data is illustrated in FIG. 2.

### Immunofluorescence

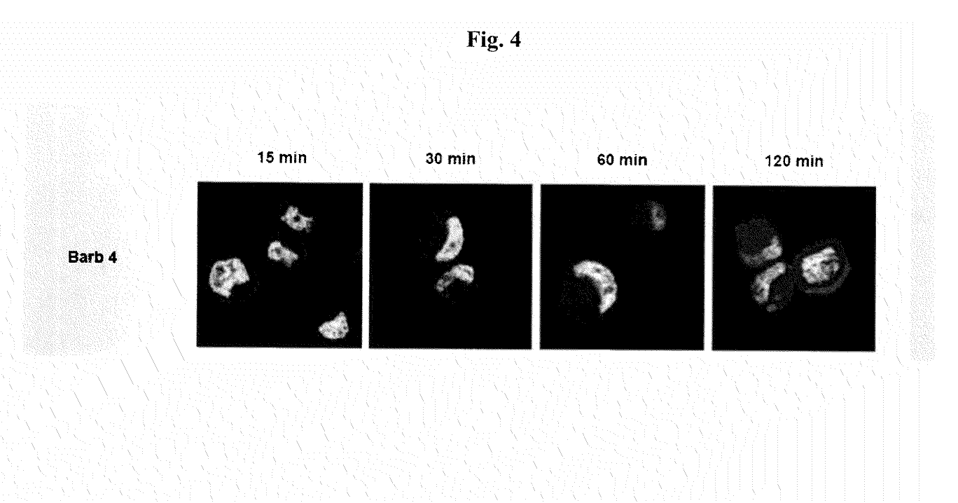

Endocytosis was determined for BARB4 on human pancreas carcinoma cell-line BXPC-3. BARB4 antibody (purified) was conjugated with Fluorescent orange 548 reactive (Fluka, Buchs, Switzerland). Conjugated BARB4 antibody at a final concentration of 40 μg/ml was directly given to 1×10⁶ cells and incubated for indicated times at 37° C. Cells were harvested, rinsed and resuspended in phosphate buffer saline pH 7,4 (PBS). 100 μl of each cell suspension was fixed on slides. Finally the slides were mounted with Fluorescent Mounting Medium (DakoCytomation, Carpinteria, USA) and analyzed by confocal microscopy. The data is illustrated in FIG. 4.

### Glycosidase Assay on Cytospins

To determine whether O- or N-linked sugar residues are potentially involved in BARB4 binding to tumor cells, cytospin preparations of BXPC-3 cancer cells were incubated with N- or O-glycosidase. In brief, 4×10⁵ human pancreas carcinoma cells (BXPC-3) were resuspended in 1 ml Dulbecco's phosphate buffered saline pH 7.2 (Sigma, Taufkirchen, Germany) and incubated with 10 U/ml N-glycosidase or 40 mU/ml O-glycosidase (both Roche Applied Science, Mannheim, Germany) for 2 hours at 37° C. Untreated cells in Dulbecco's phosphate buffered saline served as control. Cytospins were prepared and immunohistochemical staining with biotinylated BARB4 antibody (100 μg/ml) was performed.

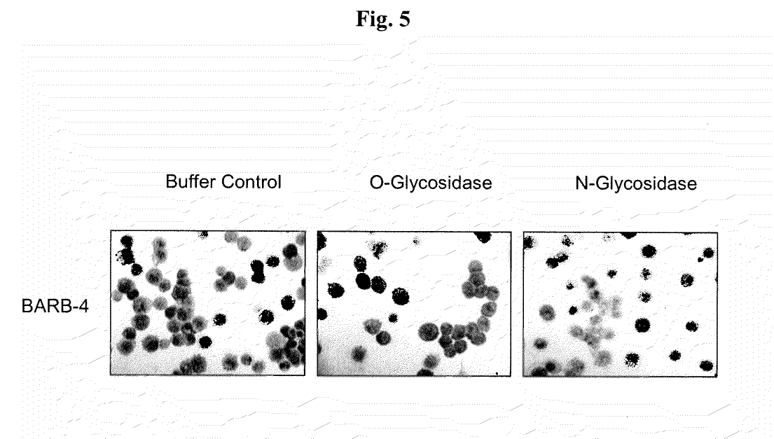

After treatment of the cells, binding of BARB4 was evaluated by immunohistochemical staining (FIG. 5). The data show a clear reduction of surface binding of BARB4 on cells treated with N-glycosidase, while treatment with O-glycosidase has no effect on BARB4 binding. BARB4 could therefore bind to an eptiope that includes or consists of a carbohydrate moiety that is removed or modified by O-glycosidase treatment.

### Cell Culture

For BARB4 target assays the established, human pancreatic adenocarcinoma cell line BXPC-3 was used. The adherent cells were grown to a confluence of about 90% in RPMI 1640 culture medium (PAA, Vienna) supplemented with 10% (V/V) FCS, 1% (V/V) glutamine and 1% (V/V) streptomycin/penicillin at 37° C. and a 7% CO2 atmosphere (HERA cell incubator, Heraeus, Hanau). Sterile tissue culture dishes 145/20 (greiner bio-one, Frickenhausen) were prepared with 1 ml cell suspension containing 2−3×10⁶ cells/ml (suspension medium is equivalent to culture medium described above) and 50 ml supplemented RPMI 1640 medium. After three or four days the carcinoma cells could be harvested for membrane extract preparation. For this purpose, BXPC-3 cells were washed with phosphate-buffered saline (PBS; containing 0.137 M NaCl, 0.027 M KCl, 0.065 M Na₂HPO₄×2H₂O, 0.015 M KH₂PO₄) and the adherent cells were scraped off the tissue culture dishes. The cell suspension was centrifuged (1500×g for 5 minutes at room temperature). The supernatant was removed and the pellet was washed again with PBS followed by a further centrifugation step (1500×g, 5 minutes at room temperature). Afterwards the supernatant was removed again. A single cell pellet includes carcinoma cells from 10 tissue culture dishes. It was immediately used for membrane extract preparation or stored at −20° C.

### Membrane Protein Extraction

The pelleted carcinoma cells were re-swollen for 30 minutes on ice in 10 ml buffer (20 mM HEPES pH 7.4, 3 mM KCl, 3 mM MgCl₂) containing a protease inhibitor tablet (Complete mini tablet, Roche Diagnostics, Mannheim). The cell suspension was sonicated (Sonifikator Labsonic V, B. Braun, Melsungen) on highest intensity for 5 minutes at 4° C. Subsequently the suspension was centrifuged with 13,000 rpm for 10 minutes at 4° C. The obtained pellets containing the cell nuclei were removed, while the supernant with remaining proteins was centrifuged under vacuum in an ultracentrifuge (Beckmann, München) with 40,000 rpm for 45 minutes at 10° C. After ultra centrifugation the soluble cell proteins were found in the supernant while the pellet contains the membrane proteins. The pellet with the membrane protein fraction was washed with 1 ml puffer (20 mM HEPES pH 7.4, 3 mM KCl, 3 mM MgCl₂). Then the pellet was dissolved in 1 ml lysis buffer (50 mM Tris/HCl, pH 7.4, 1% (w/V) Nonidet P40, 0.25% (w/V) Na-Deoxycholate, 150 mM NaCl, 1 mM EDTA; 1 μg/ml Pepstatin and a Complete mini tablet) at least for one hour. Insoluble residues were removed by centrifugation with 13,000 rpm for 10 minutes at 4° C. The membrane protein lysat were stored at −20° C.

### Antibody-Sepharose Coupling

BARB4 antibody was coupled to a cyanogen bromide-activated-Sepharose® 4 Fast Flow matrix (Sigma-Aldrich, Steinheim). 50 mg antibody was mixed with 20 ml coupling buffer (0.1 M NaHCO₃, pH 8.3, 0.5 M NaCl). Then 2.5 g activated matrix was washed and re-swollen with 500 ml ice cold HCl (1 mM, pH 2,6) for 40 min to remove lactose. Wash-steps with 100 ml distilled water and 12.5 ml coupling buffer followed. To avoid the active groups hydrolyze in basic buffer, ligand coupling buffer solution was immediately transferred to the activated Sepharose® matrix. The suspension was mixed over night at 4° C. Coupling buffer containing non-reacted antibody was eliminated. The affinity matrix was washed again with 250 ml fresh coupling buffer. Non-reacted, activated groups were blocked with 0.2 M glycine buffer (pH 8) over night at 4° C. After removing block buffer, the beads were first washed with coupling buffer, then with 0.1 M acetate buffer (pH 4). The wash cycle of high and low buffer solutions was done 5 times each with 100 ml solution. The affinity chromatographic matrix was filled in a column, stored in a PBS solution supplemented with 0.05% sodium acid (pH7.2) at 4° C.

### Affinity Chromatography

For membrane protein purification the sepharose-BARB4 column was coupled to a FPLC system (Pharmacia, Freiburg). Membrane extracts were added to the column and unbound proteins were eliminated by washing the column Buffer A (PBS) for 25 min. The bound proteins were eluted with Puffer B (0.1 M Glycine pH 2.2) and neutralized immediately with 1 M Tris (pH 9.0). The eluate was stored at −20° C.

### Gel Electrophoresis

10% SDS-PAGE gels (Composition according to Sambrook et al 1989) under reducing conditions were performed using the method of Laemmli (Nature 227(5259):680 (1970)). Depending on their primary concentration, proteins were concentrated up to higher amount per ml, before using for gel electrophoresis. Therefore one part of protein solution was mixed with 3 parts of ice cold acetone. The mixture was stored at −20° C. overnight to precipitate the proteins. Afterwards the suspension was centrifuged (13,000 rpm for 10 minutes at 4° C.). The supernant was removed and the protein pellet was dried at room temperature. Loading buffer (50 mM Tris/HCl pH 6.8, 0.1% (w/V) bromphenol blue, 2% (V/V) sodiumdodecylsulfate (SDS), 10% (V/V) Glycerol) and 1 M Dithiotreithol (DTT) was added to the pellet in a ratio of 10:1. At least the solution was heated up to 95° C. for 5 minutes before starting gel electrophoresis. The SDS-PAGE was done at 20 mA for about 1.5 hours.

### Coomassie Brilliant Blue Staining

After gel electrophoresis the proteins were fixed and stained with coomassie solution (0.4% (w/V) Coomassie Brilliant Blue R250, 40% (v/v) ethanol, 7% (v/v) acetic acid in Millipore®-water) for at least 20 min. The gel was destained (25% methanol, 12.5% acetic acid in Millipore®-water) for 1 to 2 h.

### Western Blotting

SDS-PAGE separated proteins were transferred to a nitrocellulose membrane (Schleicher und Schuell, Dassel) by semidry western blotting (Towbin et. al, Proc Natl Acad Sci USA. 76:4350 (1979)). The membrane was blocked with PBS-Teween® containing 5% low fat milk powder in powder. The membrane was incubated in a primary antibody solution containing 5% low fat milk in PBS-Tween® for 1 hour. The preparation was washed three times for 10 minutes. The same procedure was done with the second, horseradish peroxidase-conjugated antibodies. A second wash step was performed (3×5 minutes). The reaction was detected using the SuperSignal chemo luminescence kit from Pierce (Perbio Science Deutschland GmbH, Bonn).

Transfection with Small Interfering RNA

Before transfection, 24-well plates were inoculated with 0.5×10⁴ BXPC-3 cells in growth medium (RPMI 1640 supplemented with 110% (V/V) FCS and 1% (V/V) glutamine). The cells were incubated at 37° C. and a 7% CO2 atmosphere over night to reach a confluence of 50 to 70% the following day. 60 minutes prior to transfection, the medium was carefully aspirated from the wells and 250 μl (300 μl for nontransfected cells) fresh growth medium was added. For each well 50 μl transfection solution was prepared. First 24.5 ¹¹ serum-free OptiMEM® I+GlutaMAX™-I (1×) medium (Invitrogen, Karlsruhe) supplemented with 0.5 μl siLentFect™Lipid Reagent (Biorad, München) was mixed. Afterwards 25 μl serum-free medium containing siGENOME SMARTpool siRNA (Dharmacon, Lafayette, USA) was prepared to achieve a final concentration of 100 nM siRNA. Both solutions were prepared separately. After five minutes they were added together and mixed by pipetting. The solution was incubated for 20 minutes at room temperature. Transfection solution was added to the cells in serum containing medium. After 24 hours the transfection medium was exchanged with normal growth medium to minimize cell toxicity. For optimal growing conditions the medium was renewed after 48 hours. To exclude nonspecific or cyto-toxic effects on protein knockdown, Silencer® negative control #1 siRNA (100 mM final concentration; Ambion, Cambridge) and non-transfected cells served as control.

### FACS Analysis

FACS assays were done with transfected cells. The cells were detached with trypsin-EDTA (1×) and resuspended in culture medium. Cells were adjusted to 2×10⁵ cells per FACS-tube (greiner bio-one, Frickenhausen). Cells were incubated on ice for 30 minutes to stimulate the expression of membrane proteins, repressed by trypsin-EDTA treatment. The cell suspensions were centrifuged (1400×g for 5 minutes) and washed with FACS buffer (BD FACSFlow™, Becton Dickinson Biosciences, San Jose). The cells were incubated with primary antibody for 20 minutes on ice. Afterwards the cells were centrifuged and washed again with FACS buffer. The secondary, FITC conjugated antibody was added to cells for 20 min with a dilution ratio of 1:50 and cells were incubated in the dark on ice. Cells were washed and centrifuged once again. At least 200 μl FACS buffer was added and cells were analyzed by flow cytometry (FACScan; Becton Dickinson, San Jose, Calif.) using WinMDI software.

### Example 2

This example describes isolation and identification of BARB4 target protein, an apparent isoform of TAF 15, and various studies to validate BARB4 target.

To purify BARB4 target protein, pooled membrane protein extracts were injected in the BARB4 coupled Sepharose® column. The eluate was examined by western blot analysis and SDS-PAGE followed by peptide mass fingerprinting.

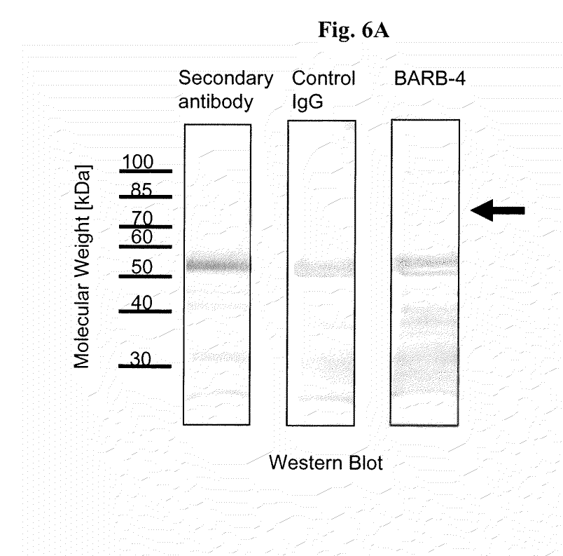

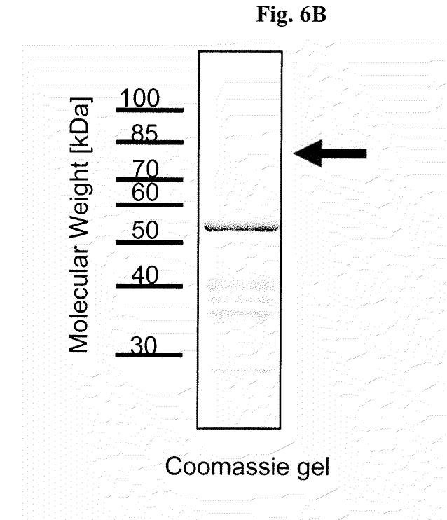

To exclude unspecific protein bindings, unrelated human serum IgG (ChromPure human IgG, Dianova, Hamburg) served as isotype control. On Western blots with membrane preparations of tumor cell line BXPC-3, BARB4 antibody binds to a membrane molecule with a relative molecular mass between 70 and 85 kD (FIG. 6A).

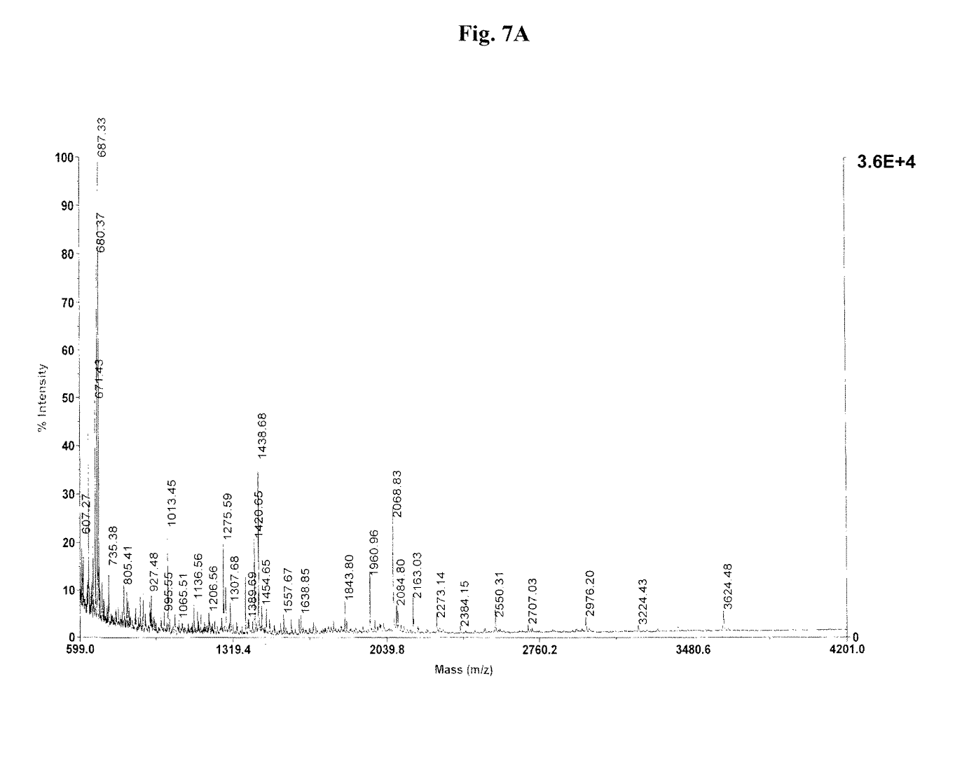

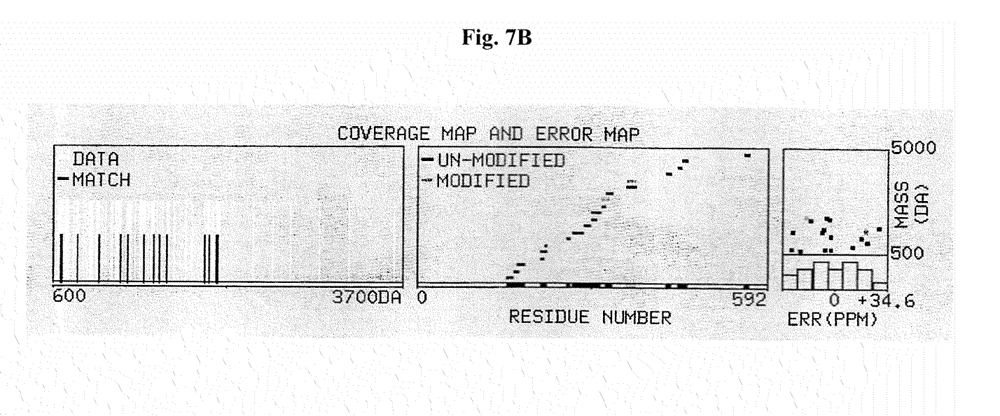

To identify the eluted protein, a Coomassie stained gel (10-fold higher concentrated by acetone, FIG. 6B) was used for peptide mass fingerprint analysis (Toplab, Proteomics-Devision, Martinsried). The corresponding SDS-PAGE protein band was reduced with DTT, alkylated with iodine acetamid and digested by trypsin over night and measured by MALDI MS (FIG. 7A). Finally, peptide masses were compared to human sequences in NCBI-data base by ProFound (FIG. 7B). Based upon these studies, BARB4 target is apparently an isoform of human TAF15.

To validate BARB4 target protein TAF™ 5, detection of TAF 15 protein was subjected to knockdown and FACS analysis. In brief, BXPC-3 cells were transfected with siGENOME SMARTpool TAF 15 siRNA, and Silencer® negative control #1 siRNA were harvested 48 hours after transfection. Nontransfected cells served as control. Cells were incubated with BARB4 antibody (300 μg/ml) for 20 minutes on ice.

Mouse anti-human CD55 antibody (DAF; 1:1000; Acris, Hiddenhausen) was used to control protein expression of other cell surface membrane proteins. The samples were incubated with secondary, FITC labeled antibodies (rabbit anti-human IgG, Dako, Hiddenhausen and rabbit anti-mouse IgG, Dianova, Hamburg) for 20 minutes on ice. The cells were analyzed by flow cytometry (FACScan, Becton Dickinson, San Jose) and evaluated with WinMDI software.

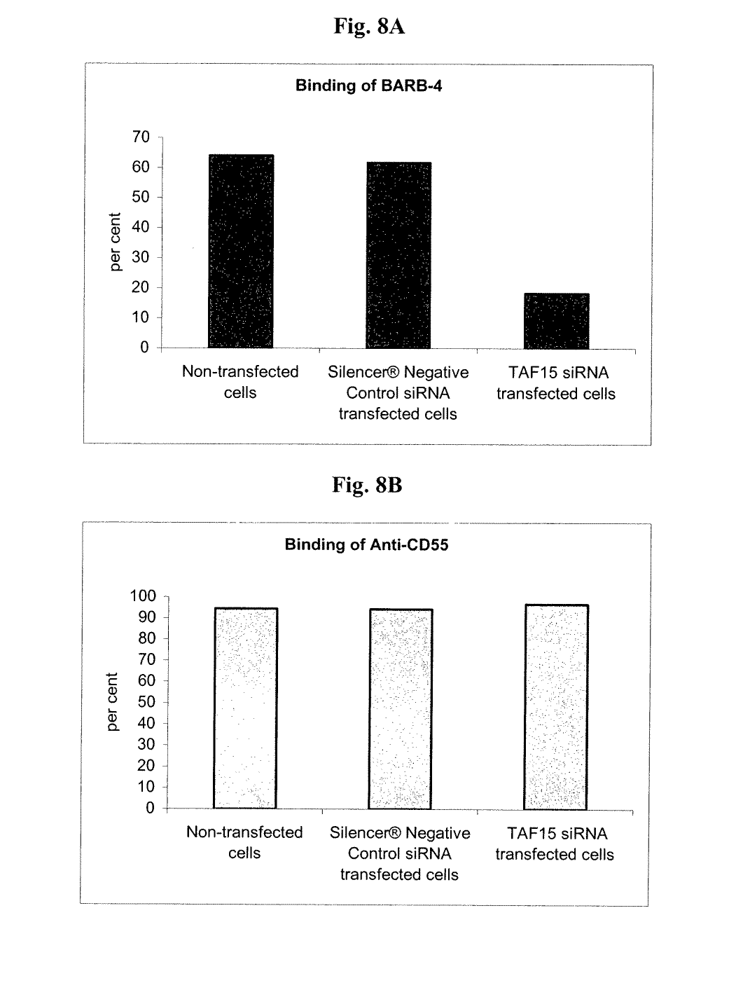

FACS analysis demonstrates that BARB4 antibody binds to untreated cells and on cells treated with unrelated siRNA (negative control) after 48 h (FIG. 8). Likewise a strong binding was observed with the anti-CD55 control (FIG. 8B), indicating that the silencing did not affect the expression of other cell surface membrane molecules. On cells treated with siRNA against human TAF15 mRNA, FACS analysis with antibody BARB4 shows clearly reduced binding (FIG. 8A). The control with anti CD55 shows again that silencing did not affect the expression of other surface membrane molecules.

To further validate BARB4 target protein TAF 15, BARB4 and a commercial TAF 15 antibody (anti-human TAFII p68, Santa Cruz Biotechnology, sc-81121) were analyzed for binding to BxPC-3, HEK 293, A549 and HeLa cell lines. In brief, cells were trypsinized with cell dissociation solution (Sigma C5789), resuspended in complete medium (RPMI 1640, PAA, E15-039, 10% Fetal bovine serum, PAA, A15-151 and 1% Glutamine, PAA, M11-004) and set on 2×10⁵/ml. After 30 minutes on ice, cells were dispersed at 1 ml per FACS-tube and washed once with ice-cold PBS by centrifugation with 500 g and 4° C. Staining was done with BARB4 (100 ug/ml) or the commercial TAF 15 (25 μg/ml) IgG antibodies, control IgGs (IgG lambda, or mouse IgG), or without antibody in 200 μl PBS. Cells were incubated with 25 μg/ml antibody in 100 μl for 30 minutes on ice, then washed with ice cold PBS and secondary antibody (anti mouse IgG-FITC, dianova 115-095-008) was applied at a dilution of 1:50 in 200 μl per tube. After another 30 minutes of incubation in the dark, cells were washed twice with PBS and analyzed by FACS. FACS analysis revealed binding of BARB4 and the TAF 15 antibodies to the surface of all four cell lines, confirming that as with BARB4 target, TAF 15 is present on the surface of various cancer and transformed (immortalized) cell lines. BARB4 target protein TAF 15 validation was then analyzed by immunoprecipitation of MKNcell extracts with BARB4 and subsequent Western blotting with either BARB4 or anti-TAF 15 antibodies (Biomol, A300-308A; or Aviva, ARP30111_T100).

In brief, immunoprecipitation was performed with μ Columns and μMACS Separator (Miltenyi Biotec GmbH, Bergisch Gladbach, Germany). To 750 μL membrane preparation (1.3 μg/mL) 10 μL of monoclonal human antibody (BARB4) and 50 μL of Protein G Micro Beads (magnetic labeled) were added and was filled with lysis buffer to a total volume of 900 μL. The suspension was incubated for 30 minutes rotating with 16 rpm at 4° C.

Miltenyi μColumns were placed in the magnetic field of the μMACS Separator. Columns were prepared by rinsing with 200 μL of lysis buffer (TNX buffer: 50 mM, 50 mM EDTA, 150 mM NaCl, 1% Triton, pH 7.5). Cell lysate was applied onto the column. After the lysate ran through the columns were washed with 5×200 μL lysis buffer. For elution 20 μL of pre-heated (95° C.) 1×SDS gel loading buffer (50 mM Tris HCl, pH 6.8; 50 mM DTT; 1% SDS; 0.005% bromphenol blue; 10% glycerol) was applied onto the column and incubated for 5 minutes at room temperature. A fresh collection tube was placed under the column and the column was eluted with another 50 μL of pre-heated (95° C.) 1×SDS gel loading buffer.

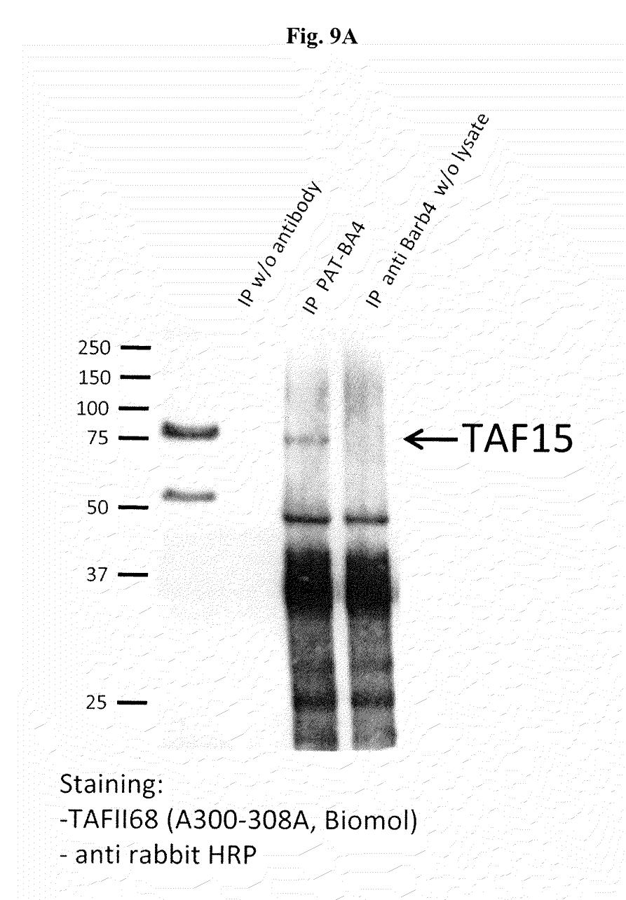

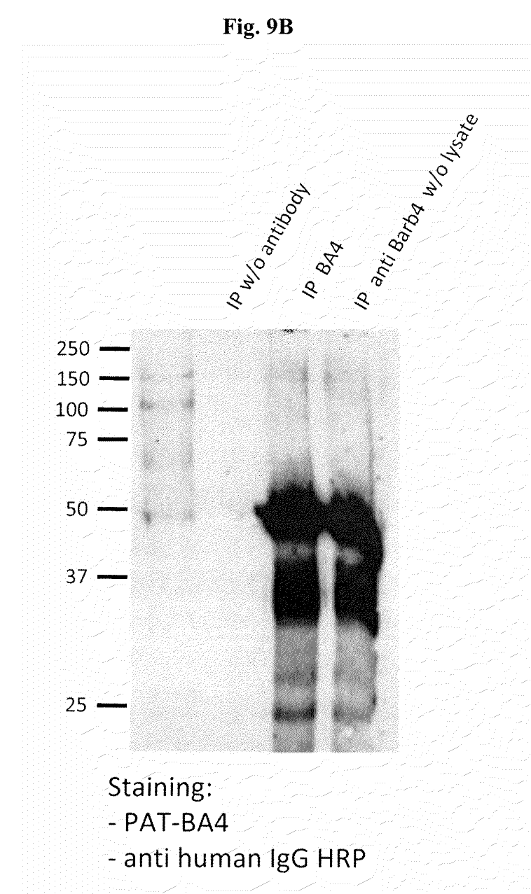

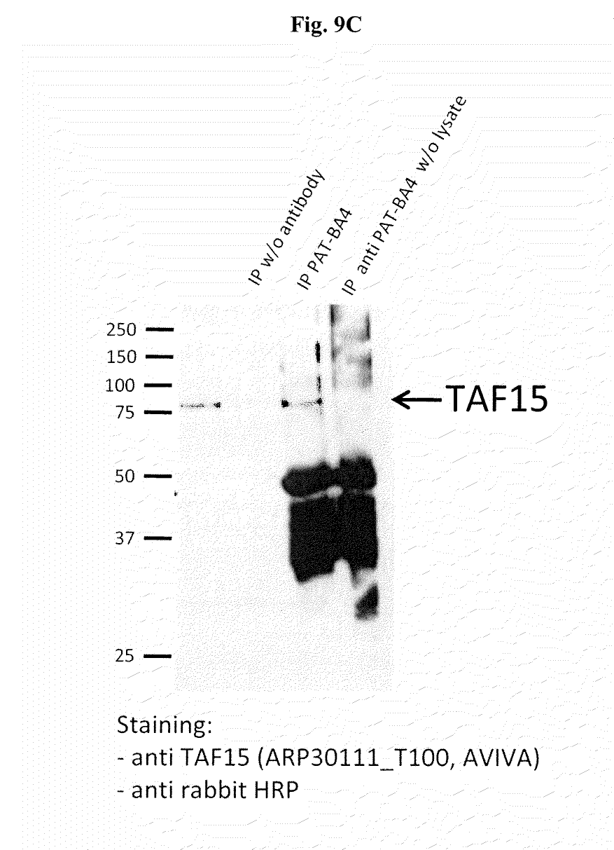

The data demonstrated that BARB4 was able to immunoprecipiate TAF 15 after staining with anti-TAF 15 antibodies (FIGS. 9A and 9C). Staining with BARB4 antibody did not appear to detect TAF15, although the assay conditions may not be optimized for (FIG. 9B).

### Example 3

This example describes cloning of TAF15 isoform from two different tumor cell lines.

A549 and HeLa cell cDNA was prepared and polymerase chain reaction (PCR) was performed using TAF 15 specific primers. The reaction products were fractionated on an agarose gel and two products migrating at around 1200 and 1800 base pairs were identified. The DNA was eluted from the gel and cloned into a vector and the sequence of each clone analyzed. The sequence information revealed sequence identity of the 1800 bp product to TAF 15 Isoforms I (1776 base pairs, 592 amino acids) and II (1767 base pairs, 589 amino acids) and the 1200 bp product was a splice variant of TAF 15 with a c-terminal deletion. Thus, it appears that these two cell lines express TAF 15 or a variant of TAF 15 with a c-terminal deletion.

1.-5. (canceled)

6. An isolated or purified antibody or functional fragment thereof that binds to a cell or to an antigen that intact BARB4 antibody, as represented by antibody produced by hybridoma DSMZ Deposit No. DSM ACC2876, or heavy and light chain sequences set forth as SEQ ID NOs:1 and 9, binds.

7. The isolated or purified antibody or functional fragment of claim 6, wherein the antibody or functional fragment binds to an adenocarcinoma cell or a squamous cell carcinoma to which BARB4 antibody, as represented by antibody produced by hybridoma DSMZ Deposit No. DSM ACC2876, or heavy and light chain sequences set forth as SEQ ID NOs:1 and 9, binds.

8. The isolated or purified antibody or functional fragment of claim 6, wherein the antibody or functional fragment binds to one or more of a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which BARB4 antibody, as represented by antibody produced by hybridoma DSMZ Deposit No. DSM ACC2876, or heavy and light chain sequences set forth as SEQ ID NOs:1 and 9, binds.

9. The isolated or purified antibody or functional fragment of claim 6, wherein the antibody or functional fragment binds to a human adenocarcinoma, squamous cell carcinoma, carcinoid carcinoma, ivasive ductal carcinoma, germ cell carcinoma of stomach, lung, colon, pancreas, esophagus, prostate, breast or testis to which BARB4 antibody, as represented by antibody produced by hybridoma DSMZ Deposit No. DSM ACC2876, or heavy and light chain sequences set forth as SEQ ID NOs:1 and 9, binds.

10. The isolated or purified antibody or functional fragment of claim 6, wherein the antibody or functional fragment binds to a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201) that intact BARB4 antibody binds.

11. An isolated or purified antibody or a functional fragment thereof comprising a heavy or a light chain variable region sequence with about 60% or more identity to a heavy or light chain sequence variable regions as set forth in SEQ ID NOs:1 and 9.

12.-15. (canceled)

16. The antibody or function fragment of claim 6, wherein the antibody or functional fragment inhibits or reduces proliferation, or stimulates or induces apoptosis, of one or more of a stomach adenocarcinoma cell, a lung adenocarcinoma cell, a pancreas adenocarcinoma cell, a colon adenocarcinoma cell, a breast adenocarcinoma cell, or an esophagus squamous cell carcinoma, a human adenocarcinoma, human squamous cell carcinoma, human carcinoid carcinoma, human invasive ductal carcinoma, or a human germ cell carcinoma in any of stomach, lung, colon, pancreas, esophagus, prostate, breast or testis, or a pancreas cancer cell line BXPC-3 (ATCC Deposit No. CRL-1687), a colon cancer cell line HT-29 (ATCC Deposit No. HTB-38) or a stomach cancer cell line 23132/87 (DSMZ Deposit No. ACC 201).

17. The antibody or functional fragment of claim 6, wherein the antibody is polyclonal or monoclonal.

18. The antibody or functional fragment of claim 6, wherein the antibody is selected from IgG, IgA, IgM, IgE and IgD.

19. (canceled)

20. The antibody or functional fragment of claim 6, wherein the antibody or functional fragment has a binding affinity within about 1-5000 fold of the binding affinity of BARB4 antibody, as represented by antibody produced by hybridoma DSMZ Deposit No. DSM ACC2876, or heavy and light chain sequences set forth as SEQ ID NOs:1 and 9, for binding to a neplastic, cancer, tumor or metastatic cell.

21.-23. (canceled)

24. The antibody or functional fragment of claim 6, wherein the antibody or functional fragment has a binding affinity within about KD 10⁻⁵ M to about KD 10⁻¹³ M for binding to a neplastic, cancer, tumor or metastatic cell.

25.-27. (canceled)

28. The antibody or functional fragment of claim 6, wherein the functional fragment is selected from Fab, Fab′, F(ab′)₂, Fv, Fd, single-chain Fv (scFv), disulfide-linked Fvs (sdFv), V_{L} and V_{H} domain fragments, trispecific (Fab₃), bispecific (Fab₂), diabody ((V_{L}-V_{H})₂ or (V_{H}-V_{L})₂), triabody (trivalent), tetrabody (tetravalent), minibody ((scFv-C_{H}3)₂), bispecific single-chain Fv (Bis-scFv), IgGdeltaCH2, scFv-Fc and (scFv)₂-Fc.

29.-31. (canceled)

32. The antibody or functional fragment of claim 6, wherein the cell or cell line expresses a cell membrane bound TATA-binding protein-associated factor 15 (TAF 15 polypeptide), or the antigen comprises a cell membrane bound TATA-binding protein-associated factor 15 (TAF 15 polypeptide).

33. (canceled)

34. The antibody or functional fragment of claim 11, wherein the antibody or functional fragment competes with BARB4 antibody, as represented by DSMZ Deposit No. DSM ACC2876, or heavy and light chain sequences set forth as SEQ ID NOs:1 and 9, for binding to TATA-binding protein-associated factor 15 (TAF15 polypeptide).

35. A heavy or light chain sequence comprising SEQ ID NO:1, 3, 5, 7 or 9.

36. A host cell that expresses the antibody or functional fragment of claim 6, or the heavy or light chain sequence of SEQ ID NO:1, 3, 5, 7 or 9.

37.-48. (canceled)

49. A pharmaceutical composition comprising the antibody or functional fragment of claim 6, or the heavy or light chain sequence of SEQ ID NOs:1, 3, 5, 7 or 9, and a pharmaceutically acceptable carrier or excipient.

50.-63. (canceled)

64. An isolated or purified TATA-binding protein-associated factor 15 (TAF 15 polypeptide), wherein the TAF 15 polypeptide is a cell membrane bound isoform.

65. An isolated or purified TATA-binding protein-associated factor 15 (TAF 15 polypeptide), wherein the TAF 15 polypeptide includes a carbohydrate moiety.

66. (canceled)

67. The isolated or purified antigen or TATA-binding protein-associated factor 15 (TAF 15 polypeptide) of claims 64, or 65, wherein the antigen or TAF 15 polypeptide has a molecular weight of about 70-85 KDa, as determined by sodium dodecyl sulfate polyacrylamide gel electrophoresis (SDS-PAGE).

68. The isolated or purified antigen or TATA-binding protein-associated factor 15 (TAF 15 polypeptide) of claims 64, or 65, wherein the antigen or TAF 15 polypeptide has a sequence identical to a sequence in SEQ ID NO:11 or 12.

69.-74. (canceled)

75. An isolated or purified antibody or subsequence thereof that binds to the antigen or TATA-binding protein-associated factor 15 (TAF 15 polypeptide) of claims 64, or 65.

76. The antibody or subsequence thereof of claim 75, wherein binding of the antibody or subsequence thereof to the antigen or TAF 15 polypeptide after treatment with an N-glycosidase is reduced as compared to binding of the antibody or subsequence thereof to the untreated antigen or TAF 15 polypeptide.

77. The antibody or subsequence thereof of claim 75, wherein binding of the antibody or subsequence thereof to the antigen or TAF 15 polypeptide expressed on cells after transfection with an antisense nucleic acid of TAF 15 is reduced as compared to binding of the antibody or subsequence thereof to the antigen or TAF 15 polypeptide expressed on cells not transfected with the antisense nucleic acid.

78. The antibody or subsequence thereof of claim 75, wherein the antibody or subsequence thereof competes with BARB4 antibody (as represented by antibody produced by hybridoma DSMZ Deposit No. DSM ACC2876, or heavy and light chain sequences set forth as SEQ ID NOs:1 and 9, respectively) for binding to the TAF 15 polypeptide.

79. The antibody or subsequence thereof of claim 75, wherein the antibody or subsequence thereof is a mammalian antibody.

80. The antibody or subsequence thereof of claim 75, wherein the antibody or subsequence thereof is a human or humanized antibody.

81. The antibody or subsequence thereof of claim 75, wherein the antibody has a binding affinity for TAF 15 polypeptide expressed by a tumor or cancer cell greater than the binding affinity for TAF 15 polypeptide expressed in a non-tumor or non-cancer cell.

82.-144. (canceled)
